(19) United States
(12) Patent Application Publication
(10) Pub. No.: US 2023/0279244 A1
(43) Pub. Date: Sep. 7, 2023

(54) RESIN COMPOSITION, COATED AND DRIED PRODUCT, MELT-KNEADED PRODUCT, OPTICAL FILTER, IMAGE DISPLAY DEVICE, SOLID-STATE IMAGING ELEMENT, SQUARYLIUM COMPOUND, AND METHOD FOR PRODUCING THE SAME

(71) Applicant: FUJIFILM Corporation, Tokyo (JP)

(72) Inventors: Daisuke SASAKI, Shizuoka (JP); Hiroki KUWAHARA, Shizuoka (JP); Nobutaka FUKAGAWA, Shizuoka (JP)

(73) Assignee: FUJIFILM Corporation, Tokyo (JP)

(21) Appl. No.: 18/314,793

(22) Filed: May 9, 2023

Related U.S. Application Data
(63) Continuation of application No. PCT/JP2021/048224, filed on Dec. 24, 2021.

(30) Foreign Application Priority Data
Dec. 25, 2020 (JP) 2020-217497
Dec. 2, 2021 (JP) 2021-196123

(51) Int. Cl. C09D 7/41; C08K 5/29; C09D 125/06; C09D 145/00; C09D 133/10; C07C 251/24; C07F 17/02; C09B 57/00; G02F 1/1335

(52) U.S. Cl. CPC C09D 7/41; C08K 5/29; C09D 125/06; C09D 145/00; C09D 133/10; C07C 251/24; C07F 17/02; C09B 57/007; G02F 1/133509; G02F 2203/11

(57) ABSTRACT

Provided are a resin composition containing a squarylium compound and a resin, in which the squarylium compound includes at least one selected from squarylium compounds represented by specific formulae; a coated and dried product or a melt-kneaded product; an optical filter including these components; an image display device and a solid-state imaging element including the optical filter; and a squarylium compound represented by a specific formula and a method for producing the same.

### CROSS-REFERENCE TO RELATED APPLICATIONS

This application is a Continuation of PCT International Application No. PCT/JP2021/048224 filed on Dec. 24, 2021, which claims priority under 35 U.S.C. § 119 (a) to Japanese Patent Application No. 2020-217497 filed in Japan on Dec. 25, 2020, and Japanese Patent Application No. 2021-196123 filed in Japan on Dec. 2, 2021. Each of the above applications is hereby expressly incorporated by reference, in its entirety, into the present application.

### BACKGROUND OF THE INVENTION

### 1. Field of the Invention

The present invention relates to a resin composition suitable as a constituent material of an optical filter and the like, a coated and dried product or a melt-kneaded product, an optical filter formed of these components, and an image display device and a solid-state imaging element using the optical filter. The present invention also relates to a squarylium compound suitable as a light absorbing component of the resin composition and the like, and a method for producing the same.

### 2. Description of the Related Art

A squarylium compound is a promising compound as an optical material such as an organic coloring agent because it can absorb light having a specific wavelength. For example, applications to optical use, such as a charge generating material for an electrophotographic photo receptor (for example, JP1985-169453A (JP-S60-169453A)), a dye (for example, an electrophotographic toner dye (JP2009-036811A), and a light absorbing agent for an optical filter mounted on an image display device or the like (for example, WO2019/167930A1), have been proposed.

Among image display devices, a liquid crystal display device has a wide range of applications because it consumes less power and can save space. In the liquid crystal display device, since the liquid crystal panel itself, on which an image is displayed, is a non-light emitting element which does not emit light, a backlight unit is disposed on a rear surface of the liquid crystal panel. As a light source of the backlight unit, white light emitting diode (LED), which mixes blue light emitted from a blue LED with light emitted from a yellow phosphor or a green phosphor and a red phosphor to produce white light, has been used. For the backlight unit using such a white LED, there has been proposed a technique of blocking (absorbing) light of an unnecessary wavelength, emitted from the white LED, to improve a color reproduction range. As an optical filter (light absorbing film) which blocks (absorbs) the light of an unnecessary wavelength, various optical filters containing a coloring agent such as the squarylium compound and a resin have been proposed.

The squarylium compound is a fluorescent coloring agent having a high fluorescence quantum yield, but since the squarylium compound is easily oxidized (decomposed) by light (irradiation) and lose its function as the coloring agent, it has been considered that it is difficult to apply the squarylium compound to applications (image display device, coloring agent for ink jet, and the like) which require high light resistance maintaining blocking performance (light absorbing performance) of light having a specific wavelength even in a case of being irradiated with light.

As an optical filter for improving such a decrease in light resistance, for example, WO2019/167930A1 proposes an optical filter manufactured using a resin composition which contains a compound represented by a specific general formula, having a specific squarylium compound structural part and a metallocene structural part, and a resin.

### SUMMARY OF THE INVENTION

In a case where a resin solution in which a general squarylium compound and a resin are dissolved in an organic solvent is used for film formation, a state of the film formation and a state of existence of the squarylium compound are likely to vary (to be unevenness), resulting in problem of impairing the light absorbing performance of the optical filter.

An object of the present invention is to provide an optical filter which can highly absorb (block a passage of) target light having a specific wavelength, such as light of an unnecessary wavelength in incidence light, and has excellent light resistance. Another object of the present invention is to provide a resin composition suitable as a forming material for the optical filter or the like; a coated and dried product or a melt-kneaded product; and a squarylium compound suitable as a light absorbing component of the resin composition, the coated and dried product, or the melt-kneaded product, and a method for producing the squarylium compound. Still another object of the present invention is to provide an image display device and a solid-state imaging element including the optical filter.

As a result of intensive studies in view of the above-described problems, the present inventors have found that, although a squarylium compound having a specific chemical structure represented by Formula (1) or Formula (3) has a betaine structure in the molecule, the squarylium compound exhibits sufficient solubility in an organic solvent used in forming the optical filter while suppressing association due to high planarity of the squarylium compound. In a case where further studies have been carried out based on the finding, it has been found that, in a case where a film is formed by dissolving, in an organic solvent, the above-described resin composition in which the squarylium compound and the resin are used in combination, it is possible to form a coated and dried product (film-like body or the like) in which variation in a state of the film formation and a state of existence of the squarylium compound is suppressed, and the obtained film-like body (optical filter) can selectively and effectively absorb the target light having a specific wavelength, and exhibits excellent light resistance of maintaining a high degree of light absorbing performance even in a case of being irradiated with light. Furthermore, same as the coated and dried product, it has been found that a melt-kneaded product obtained by melt-kneading the squarylium compound and the resin can also selectively and effectively absorb the light having a specific wavelength, and also exhibits excellent light resistance.

The present invention has been completed by further repeating studies on the basis of the above-described finding.

That is, the above-described objects can be achieved by the following methods.
<1> A resin composition comprising:

a squarylium compound; and

a resin,

in which the squarylium compound includes at least one selected from a squarylium compound represented by Formula (1) or a squarylium compound represented by Formula (3),

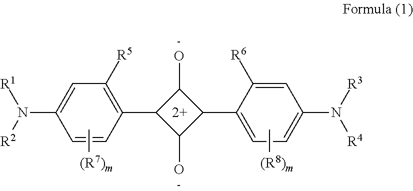

in Formula (1), R¹ to R⁴ represent an alkyl group or an aryl group, which may have a substituent, where at least one of R¹, . . . , or R⁴ is an aryl group and at least one of R¹, . . . , or R⁴ is an alkyl group, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3,

here, the squarylium compound represented by Formula (1) has at least one branched alkyl group having 4 or more carbon atoms,

Dye-(Q¹)ₙ₁ Formula (3)

in Formula (3), Dye represents a structural part obtained by removing n1 hydrogen atoms from a squarylium compound represented by Formula (4), Q¹ represents a group represented by Formula (4M), and n1 is an integer of 1 to 6,

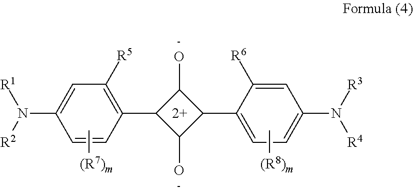

in Formula (4), R¹ to R⁴ represent an alkyl group or an aryl group, which may have a substituent, where at least one of R¹, . . . , or R⁴ is an aryl group and at least one of R¹, . . . , or R⁴ is an alkyl group, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3,

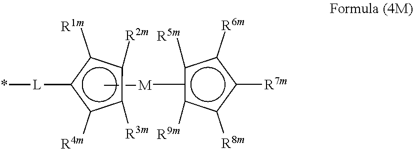

in Formula (4M), L represents a single bond or a divalent linking group which is not conjugated with Dye, R^{1m} to R^{9m} represent a hydrogen atom or a substituent, M represents Fe, Co, Ni, Ti, Cu, Zn, Zr, Cr, Mo, Os, Mn, Ru, Sn, Pd, Rh, V, or Pt, and * represents a bonding part with Dye.
<2> The resin composition according to <1>,

in which the squarylium compound represented by Formula (1) is represented by Formula (2),

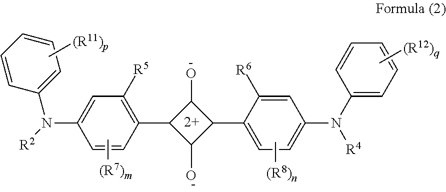

in Formula (2), R² and R⁴ represent an alkyl group, R¹¹ and R¹² represent a substituent, p and q represent an integer of 0 to 5, and R⁵ to R⁸, m, and n have the same meaning as R⁵ to R⁸, m, and n in Formula (1),

here, the squarylium compound represented by Formula (2) has at least one branched alkyl group having 4 or more carbon atoms.
<3> The resin composition according to <1> or <2>,

in which at least one of R², R⁴, R⁹, or R¹⁰ contains a branched alkyl group having 4 or more carbon atoms.
<4> The resin composition according to <1>,

in which the squarylium compound represented by Formula (4) is represented by Formula (5),

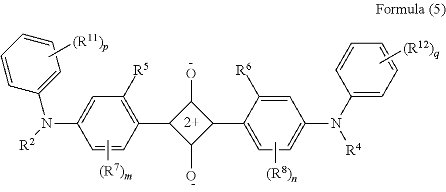

in Formula (5), R² and R⁴ represent an alkyl group, R¹¹ and R¹² represent a substituent, p and q represent an integer of 0 to 5, and R⁵ to R⁸, m, and n have the same meaning as R⁵ to R⁸, m, and n in Formula (4).
<5> The resin composition according to <1> or <4>,

in which the squarylium compound represented by Formula (4) or the squarylium compound represented by Formula (5) has at least one branched alkyl group having 4 or more carbon atoms.
<6> The resin composition according to <1>, <4>, or <5>,

in which M in Formula (4M) is Fe.
<7> The resin composition according to any one of <1> to <6>,

in which a glass transition temperature of the resin is −80° C. to 200° C.
<8> The resin composition according to any one of <1> to <7>,

in which the resin is at least one selected from a polystyrene resin, a cellulose acylate resin, a poly(meth)acrylic resin, a polyester resin, a cycloolefin resin, or a polycarbonate resin.
<9> The resin composition according to any one of <1> to <8>, further comprising:

a solvent having a boiling point of 200° C. or lower,

in which the resin and the squarylium compound are dissolved in the solvent.
<10> A coated and dried product obtained by applying and drying the resin composition according to <9> on a substrate.
<11> A melt-kneaded product of the resin composition according to any one of <1> to <8>.
<12> An optical filter comprising:

the resin composition according to any one of <1> to <7>; and

the coated and dried product according to <10> or the melt-kneaded product according to <11>.
<13> The optical filter according to <12>,

in which the optical filter has a film form.
<14> An image display device comprising:

the optical filter according to <12> or <13>.
<15> A solid-state imaging element comprising:

the optical filter according to <12> or <13>.
<16> A squarylium compound represented by Formula (1) or Formula (3),

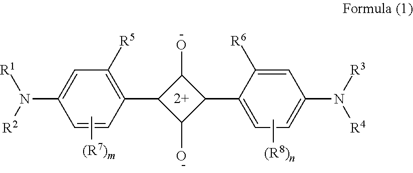

in Formula (1), R¹ to R⁴ represent an alkyl group or an aryl group, which may have a substituent, where at least one of R¹, . . . , or R⁴ is an aryl group and at least one of R¹, . . . , or R⁴ is an alkyl group, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3,

here, the squarylium compound represented by Formula (1) has at least one branched alkyl group having 4 or more carbon atoms,

Dye-(Q¹)ₙ₁ Formula (3)

in Formula (3), Dye represents a structural part obtained by removing n1 hydrogen atoms from a squarylium compound represented by Formula (4), Q¹ represents a group represented by Formula (4M), and n1 is an integer of 1 to 6,

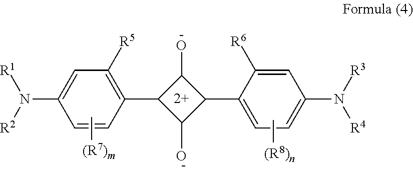

in Formula (4), R¹ to R⁴ represent an alkyl group or an aryl group, which may have a substituent, where at least one of R¹, . . . , or R⁴ is an aryl group and at least one of R¹, . . . , or R⁴ is an alkyl group, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3,

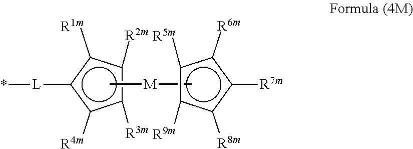

in Formula (4M), L represents a single bond or a divalent linking group which is not conjugated with Dye, R^{1m} to R^{9m} represent a hydrogen atom or a substituent, M represents Fe, Co, Ni, Ti, Cu, Zn, Zr, Cr, Mo, Os, Mn, Ru, Sn, Pd, Rh, V, or Pt, and * represents a bonding part with Dye.
<17> The squarylium compound according to <16>,

in which the squarylium compound represented by Formula (1) is represented by Formula (2),

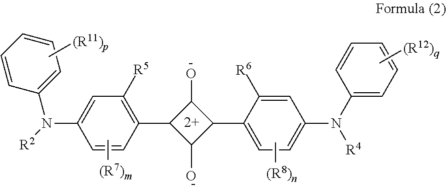

in Formula (2), R² and R⁴ represent an alkyl group, R¹¹ and R¹² represent a substituent, p and q represent an integer of 0 to 5, and R⁵ to R⁸, m, and n have the same meaning as R⁵ to R⁸, m, and n in Formula (1),

here, the squarylium compound represented by Formula (2) has at least one branched alkyl group having 4 or more carbon atoms.
<18> The squarylium compound according to <16>,

in which the squarylium compound represented by Formula (4) is represented by Formula (5),

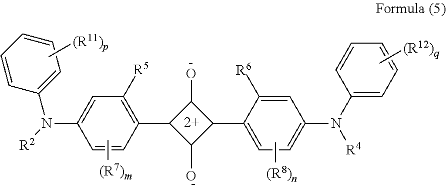

in Formula (5), R² and R⁴ represent an alkyl group, R¹¹ and R¹² represent a substituent, p and q represent an integer of 0 to 5, and R⁵ to R⁸, m, and n have the same meaning as R⁵ to R⁸, m, and n in Formula (4).
<19> A method for producing a squarylium compound, comprising:

reacting a compound represented by Formula (A) with squaric acid or a compound represented by Formula (B) to produce a squarylium compound represented by Formula (1),

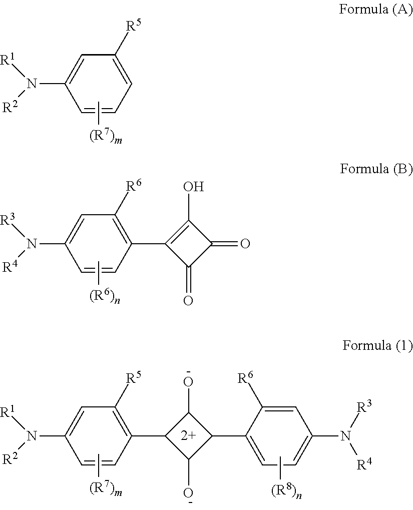

in Formula (A), Formula (B), and Formula (1), R¹ to R⁴ represent an alkyl group or an aryl group, which may have a substituent, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3,

here, in the compound represented by Formula (A) to be reacted with the squaric acid, at least one of R¹ or R² is an aryl group, at least one of R¹ or R² is an alkyl group, and the compound represented by Formula (A) has at least one branched alkyl group having 4 or more carbon atoms,

in the compounds represented by Formula (A) or Formula (B) to be reacted with each other, at least one of R¹, . . . , or R⁴ is an aryl group, at least one of R¹, . . . , or R⁴ is an alkyl group, and the compound represented by Formula (A) or Formula (B) has at least one branched alkyl group having 4 or more carbon atoms, and

the squarylium compound represented by Formula (1) has at least one branched alkyl group having 4 or more carbon atoms.

According to the present invention, it is possible to provide an optical filter which can highly absorb (block a passage of) target light having a specific wavelength, such as light of an unnecessary wavelength in incidence light, and has excellent light resistance. In addition, according to the present invention, it is possible to provide a resin composition suitable as a forming material for the optical filter or the like; a coated and dried product or a melt-kneaded product; and a squarylium compound suitable as a light absorbing component of these components, and a method for producing the squarylium compound. Furthermore, according to the present invention, it is possible to provide an image display device and a solid-state imaging element including the optical filter.

The above-described and other features and advantages of the present invention will become more apparent from the following description, appropriately referring to the accompanying drawing.

### BRIEF DESCRIPTION OF THE DRAWINGS

FIG. 1 is a schematic diagram showing an outline of an embodiment of a liquid crystal display device provided with an optical filter according to the present invention.

### DESCRIPTION OF THE PREFERRED EMBODIMENTS

In compounds (coloring agents) represented by chemical structural formulae described in the present invention or the present specification, cations are present in a delocalized manner, and a plurality of tautomer structures is present. Therefore, in the present invention, in a case where at least one tautomer structure of a certain coloring agent matches a chemical structural formula defined as each general formula, the coloring agent is considered as a coloring agent represented by the individual general formula. Therefore, a coloring agent represented by a specific general formula can be said to be a coloring agent having at least one tautomer structure which can be represented by the specific general formula. In the present invention, a coloring agent represented by a general formula may have any tautomer structure as long as at least one tautomer structure of the coloring agent matches the general formula.

In the present invention, numerical ranges expressed using “to” include numerical values before and after “to” as the lower limit value and the upper limit value. In the present invention, in a case of setting a plurality of numerical ranges for a content of a compound or the like, physical properties, and the like, the upper limit value and the lower limit value which form the numerical range are not limited to any particular combination of the upper limit value and the lower limit value, and a numerical range can be formed by appropriately combining the upper limit value and the lower limit value of each numerical range.

In the present invention, in a case of a plurality of substituents, linking groups, and the like (hereinafter, referred to as a substituent and the like) represented by a specific reference numeral, or in a case of simultaneously or alternatively defining a plurality of the substituent and the like, it means that each of the substituent and the like may be the same or different from each other. The same applies to the definition of the number of substituents and the like. In a case where a plurality of the substituents and the like is near (particularly, adjacent to each other), it means that the substituents and the like may be linked to each other or condensed to form a ring.

In the present invention, the expression of a compound is used to include the compound itself, a salt thereof, and an ion thereof. In addition, it means that a part of the structure may be changed as long as the desired effect is not impaired. Examples of the salt of the compound include an acid-addition salt of the compound, formed of the compound and an inorganic acid or an organic acid, and a base-addition salt of the compound, formed of the compound and an inorganic base or an organic base. In addition, examples of the ion of the compound include ions generated by dissolving the salt of the compound in water, a solvent, or the like.

In the present specification, regarding a substituent (the same applies to a linking group) in which whether it is substituted or unsubstituted is not specified, within the range not impairing the desired effect, it means that the group may have any substituent. The same applies to a compound or a repeating unit in which whether it is substituted or unsubstituted is not specified.

In the present invention, in a case of defining a number of carbon atoms (also referred to as carbon atoms) in a group, the number of carbon atoms means the number of carbon atoms of the entire group. That is, in a case of an aspect in which the group further has a substituent, it means the total number of carbon atoms including the substituent. In this case, in a case where a certain group has a metallocene structural part (group) as a substituent, the number of carbon atoms forming the metallocene structural part is not included in the number of carbon atoms in the certain group.

In the present invention, in the case where a group can form an acyclic skeleton and a cyclic skeleton, unless described otherwise, the group includes an acyclic skeleton group and a cyclic skeleton group. For example, an alkyl group includes, unless described otherwise, a linear alkyl group, a branched alkyl group, and a cyclic (cyclo) alkyl group. In a case where a group forms a cyclic skeleton, the lower limit of the number of carbon atoms in the cyclic skeleton group is preferably 3 or more and more preferably 5 or more, regardless of the lower limit of the number of carbon atoms specifically described for the group.

In the present invention, the term “(meth)acrylic” is used to include both methacrylic and acrylic.

[Resin Composition]

The resin composition according to the embodiment of the present invention contains a squarylium compound represented by Formula (1) or Formula (3) and a resin as a binder. Each of the squarylium compound and the resin contained in the resin composition according to the embodiment of the present invention may be one kind or two or more kinds.

As represented by Formula (1) or Formula (3) described later, the squarylium compound has a squarylium structural part having absorption in a specific wavelength range of visible light, and further has a branched alkyl group having 4 or more carbon atoms or a specific metallocene structural part. As will be described later, the squarylium compound having such a structure can exhibit a high degree of light absorbing performance and excellent light resistance in an optical filter. Moreover, in a case where the squarylium compound having a metallocene structural part, which is represented by Formula (3), is excited by light absorption, the metallocene structural part suppresses decomposition of the squarylium compound, so that the light resistance can be further improved.

In addition, in a preferred aspect in which the squarylium compound represented by Formula (3) has at least one branched alkyl group having 4 or more carbon atoms, the above-described characteristics are further enhanced.

In the squarylium compound represented by Formula (1) and the squarylium compound represented by Formula (3), the decomposition of the squarylium compound can be effectively suppressed by a preferred aspect in which the squarylium compound forms an intramolecular hydrogen bond.

Therefore, the resin composition according to the embodiment of the present invention is suitable as a forming material for a member which absorbs light having a wavelength of 670 to 740 nm, for example, the optical filter according to the embodiment of the present invention (filter containing the squarylium compound and the resin), or as a forming material for a near-infrared cut filter described later.

The resin composition according to the embodiment of the present invention may be any composition as long as it contains the squarylium compound and the resin, and can be an appropriate form depending on an application, a method for manufacturing an optical filter, and the like. Examples thereof include a (simple) mixture obtained by dry-mixing the squarylium compound and the resin by a conventional method, a liquid composition which contains a solvent described later and is obtained by dissolving the squarylium compound and the resin in the solvent (obtained by wet-mixing the squarylium compound, the resin, and the solvent by a conventional method), a coated and dried product obtained by applying and drying this liquid composition (usually a molded product in a film form), and a molten mixture (also referred to as a molten solidified product) obtained by melting and mixing the squarylium compound and the resin, and then cooling and solidifying the mixture. Here, the solvent may remain in the coated and dried product as long as the effects of the present invention are not impaired, and a residual amount of the solvent can be, for example, 5% by mass or less in the coated and dried product. The coated and dried product and the melt-kneaded product are different from the simple mixture of the squarylium compound and the resin in that the resin forms a (continuous) matrix. That is, in the coated and dried product, the squarylium compound and the resin are once dissolved in the solvent to be mixed, and then the resin (including the squarylium compound) is precipitated (solidified) in the mixed state. On the other hand, in the melt-kneaded product, the squarylium compound and the resin are once melted and melt-mixed, and then the resin (including the squarylium compound) is cooled and solidified in the melt-mixed state. As will be described later, in the resin composition according to the embodiment of the present invention, particularly the liquid composition, variation in film formation and photo-oxidative decomposition of the squarylium compound can be suppressed. In addition, in the coated and dried product and the molten mixture according to the embodiment of the present invention, variation in the state of existence of the squarylium compound is suppressed so that the light absorbing performance is not impaired, and oxidative decomposition due to light irradiation is suppressed to exhibit high light resistance. Methods and conditions for coating and drying, and melt-kneading will be described later.

The resin composition according to the embodiment of the present invention, particularly the coated and dried product and the molten mixture, may be a cured product, but is preferable uncured product.

<Squarylium Compound>

The squarylium compound contained in the resin composition according to the embodiment of the present invention (also referred to as a squarylium compound according to the present invention) is a coloring agent compound represented by Formula (1) or Formula (3).

The squarylium compound represented by Formula (1) (may be referred to as a compound (1)) is a compound having a chemical structure represented by Formula (1), in which at least one branched alkyl group having 4 or more carbon atoms is introduced. On the other hand, the squarylium compound represented by Formula (3) (may be referred to as a compound (3)) is a compound in which a specific metallocene structural part is introduced into a chemical structure represented by Formula (4), and is preferably a compound in which at least one branched alkyl group having 4 or more carbon atoms is further introduced.

Both the compound (1) and the compound (3) have a sharp absorption spectrum and have a maximal absorption wavelength in a wavelength range of 670 to 740 nm, preferably in a wavelength range of 680 to 720 nm. The above-described wavelength range is near a boundary between a near infrared range and a visible range, and is a wavelength range of light which is needed to be absorbed as unnecessary light in display applications, sensor applications, and the like. Therefore, the optical filter containing the above-described compound is preferably used as a light blocking member (optical component) in a display or the like having an LED backlight, for example, as an optical filter in a case of being used in an image display device. In addition, the optical filter according to the embodiment of the present invention is preferably used as a near-infrared cut filter that corrects visibility of a solid-state imaging element which uses, as a light receiving section, a silicon photodiode sensing infrared rays.

In general, the squarylium compound is easily oxidized and decomposed by the absorption of light, and it is difficult to apply the squarylium compound to an image display device or the like, which requires high light resistance. In addition, in a case of forming a film, a solution (liquid composition) containing the squarylium compound and the resin tends to cause variation in the state of film formation, variation in the state of existence of the squarylium compound (also referred to as variation during film formation), and the like, and has problem of lowering the light absorbing performance. On the other hand, the squarylium compound according to the present invention, having a chemical structure represented by each of the following formulae, can overcome, as described above, the problem of the photo-oxidative decomposability of the squarylium compound, and at the same time, can overcome the drawback of a decrease in light absorbing performance by suppressing the variation during film formation. The reason is not clear, but is presumed as follows.

Generally, since the squarylium compound has high planarity, the squarylium compound is difficult to dissolve in an organic solvent, and even in a case of being dissolved, the squarylium compound tends to take various association forms such as an H-associate. The formation of such an associate broadens the absorption spectrum of the squarylium compound, lowers the light resistance, and can cause the variation in the state of film formation and the variation in the state of existence of the squarylium compound. However, in both of the compound (1) and the compound (3), a total of four substituents included in two disubstituted amino groups in the squarylium structural part are adopted in a combination including at least one alkyl group and at least one aryl group, from alkyl groups and aryl groups. Further, the compound (1) has at least one branched alkyl group having 4 or more carbon atoms, and the compound (3) has a specific metallocene structural part. It is considered that, by having such a structure, the compound (1) and the compound (3) are easily dissolved in an organic solvent, and even in a case of being dissolved at high concentrations, moderate steric hindrance makes it difficult to form the associate. Furthermore, it is considered that compatibility with the resin may be increased. Therefore, both compounds are capable of forming a film while suppressing the variation during film formation, and can exhibit a high degree of light absorbing performance in the optical filter while maintaining excellent light resistance. In particular, in the compound (3) having a specific metallocene structural part, the decomposition of the squarylium compound is highly suppressed, and the light resistance can be further improved. The reason for this is not clear yet, but is considered to be due to deactivation of the excited state of the compound (3) and the following reverse electron migration. That is, in a case where the compound (3) is photo-excited, the metallocene structural part having electron donor property rapidly inactivates an electron into the squarylium compound structural part corresponding to “Dye” in Formula (3) to deactivate the excited state. Therefore, the decomposition of the compound (3) due to photo-excitation can be suppressed. In addition, in the fluorescence deactivation due to an electron migration, the coloring agent tends to be in an unstable state (anion radical) in a case where electrons are accepted in excess, which accelerates decomposition of the coloring agent. However, in the compound (3), a reverse electron migration from an anion-radicalized coloring agent structural part to the metallocene structural part is also accelerated. It is considered that the above-described actions of the squarylium compound are exerted not only in the liquid composition but also in the melt-kneaded product.

In addition, with the resin composition according to the embodiment of the present invention, it is possible to manufacture an optical filter having various compound concentrations depending on the intended purpose.

(Squarylium Compound Represented by Formula (1))

First, the squarylium compound represented by Formula (1) will be described.

One aspect of the squarylium compound contained in the resin composition according to the embodiment of the present invention is a squarylium compound (1) represented by Formula (1). The compound (1) has at least one branched alkyl group having 4 or more carbon atoms. That is, the group represented by each reference numeral in Formula (1) or the substituent in the group represented by each reference numeral has at least one branched alkyl group having 4 or more carbon atoms.

The compound (1) is configured by appropriately selecting the group represented by each reference numeral in the formula from a range described below, but it is preferable to have a symmetrical structure with respect to a carbon four-membered ring (that a benzene ring included in R⁵ and a benzene ring included in R⁶ have the same chemical structure).

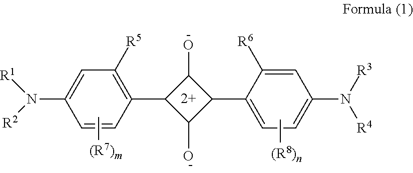

In Formula (1), R¹ to R⁴ each independently represent an alkyl group or an aryl group, which may have a substituent. However, at least one of R¹, . . . , or R⁴ is an aryl group and at least one of R¹, . . . , or R⁴ is an alkyl group. R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent. R⁷ and R⁸ represent a substituent, and m and n represent an integer of 0 to 3.

The alkyl group which can be adopted as R¹ to R⁴ may be linear, branched, or cyclic, and is preferably linear or branched and particularly preferably branched.

The number of carbon atoms in the alkyl group is not particularly limited, and is usually preferably selected from a range of 1 to 40. The lower limit thereof is more preferably 3 or more, still more preferably 5 or more, and particularly preferably 8 or more. The upper limit thereof is more preferably 35 or less and still more preferably 30 or less. Within the above-described range, the number of carbon atoms in the branched alkyl group is more preferably selected from a range of 3 to 40. In the branched alkyl group, the lower limit of the number of carbon atoms is usually still more preferably 4 or more, particularly preferably 6 or more, and most preferably 8 or more. The upper limit thereof is usually still more preferably 35 or less and particularly preferably 30 or less. However, from the viewpoint of optical properties such as light absorbing performance and light resistance, solubility in an organic solvent, and compatibility with the resin, the number of carbon atoms in the branched alkyl group is still more preferably in a range of 6 to 35, particularly preferably in a range of 8 to 30, and most preferably in a range of 8 to 24. On the other hand, from the comprehensive viewpoint including ease of synthesis (cost) and the like while maintaining the optical properties, the solubility, and the compatibility, the number of carbon atoms in the branched alkyl group is still more preferably in a range of 6 to 24, and particularly preferably in a range of 8 to 16.

The number of branches in the branched alkyl group is, for example, preferably 2 to 10 and more preferably 2 to 8.

The aryl group which can be adopted as R¹ to R⁴ may be a group having a monocyclic structure or a group having a polycyclic structure (a fused ring structure, a crosslinked ring structure, or the like), and a group having a monocyclic structure is preferable. The number of carbon atoms in the aryl group is not particularly limited, but is preferably 6 to 30, more preferably 6 to 20, still more preferably 6 to 12, and particularly preferably 6. Examples of the aryl group include groups formed of a benzene ring or a naphthalene ring, and groups formed of a benzene ring are more preferable.

Each of the alkyl group and the aryl group, which can be adopted as R¹ to R⁴, may have at least one substituent X, and in a case where a plurality of substituents X are included, adjacent substituents may be bonded to each other to form a ring structure. The number of substituents X included in one alkyl group is not particularly limited, and can be, for example, the same as p in Formula (2) described later. A position where the substituent X is bonded in the alkyl group is not particularly limited, and is appropriately determined. In addition, the number of substituents X and the position where the substituent X is bonded in one aryl group are not particularly limited, and are the same as p and q, and substituted position in Formula (2) described later.

—Substituent X—

The substituent X is not particularly limited, and examples thereof include alkyl groups (such as a methyl group, an ethyl group, a propyl group, an isopropyl group, a butyl group, a t-butyl group, an isobutyl group, a pentyl group, a hexyl group, an octyl group, a dodecyl group, and a trifluoromethyl group), cycloalkyl groups (such as a cyclopentyl group and a cyclohexyl group), alkenyl groups (such as a vinyl group and an allyl group), alkynyl groups (such as an ethynyl group and a propargyl group), aryl groups (such as a phenyl group and a naphthyl group), heteroaryl groups (such as a furyl group, a thienyl group, a pyridyl group, a pyridazyl group, a pyrimidyl group, a pyrazyl group, a triazyl group, an imidazolyl group, a pyrazolyl group, a thiazolyl group, a benzimidazolyl group, a benzoxazolyl group, a benzothiazolyl group, a quinazolyl group, and a phthalazyl group), heterocyclic groups (also called (non-aromatic) heterocyclic groups; such as a pyrrolidyl group, an imidazolidyl group, a morpholyl group, and an oxazolidyl group), alkoxy groups (such as a methoxy group, an ethoxy group, and a propyloxy group), cycloalkoxy groups (such as a cyclopentyloxy group and a cyclohexyloxy group), aryloxy groups (such as a phenoxy group and a naphthyloxy group), heteroaryloxy groups (aromatic heterocyclic oxy group), heterocyclic oxy groups (non-aromatic heterocyclic oxy groups), alkylthio groups (such as a methylthio group, an ethylthio group, and a propylthio group), cycloalkylthio groups (such as a cyclopentylthio group and a cyclohexylthio group), arylthio groups (such as a phenylthio group and a naphthylthio group), heteroarylthio groups (aromatic heterocyclic thio group), heterocyclic thio groups (non-aromatic heterocyclic thio groups), alkoxycarbonyl groups (such as methyloxycarbonyl group, an ethyloxycarbonyl group, a butyloxycarbonyl group, and an octyloxycarbonyl group), aryloxycarbonyl groups (such as a phenyloxycarbonyl group and a naphthyloxycarbonyl group), phosphoryl groups (such as dimethoxyphosphoryl and diphenylphosphoryl), sulfamoyl groups (such as an aminosulfonyl group, a methylaminosulfonyl group, a dimethylaminosulfonyl group, a butylaminosulfonyl group, a cyclohexylaminosulfonyl group, an octylaminosulfonyl group, a phenylaminosulfonyl group, and a 2-pyridylaminosulfonyl group), acyl groups (such as an acetyl group, an ethylcarbonyl group, a propylcarbonyl group, a cyclohexylcarbonyl group, an octylcarbonyl group, a 2-ethylhexylcarbonyl group, a phenylcarbonyl group, a naphthylcarbonyl group, and a pyridylcarbonyl group), acyloxy groups (such as an acetyloxy group, an ethylcarbonyloxy group, a butylcarbonyloxy group, an octylcarbonyloxy group, and a phenylcarbonyloxy group), acylamino groups (such as an acetylamino group, an ethylcarbonylamino group, a butylcarbonylamino group, an octylcarbonylamino group, and a phenylcarbonylamino group), amide groups (such as a methylcarbonylamino group, an ethylcarbonylamino group, a dimethylcarbonylamino group, a propylcarbonylamino group, a pentylcarbonylamino group, a cyclohexylcarbonylamino group, a 2-ethylhexylcarbonylamino group, an octylcarbonylamino group, a dodecylcarbonylamino group, a phenylcarbonylamino group, and a naphthylcarbonylamino group), sulfonylamide groups (such as a methylsulfonylamino group, an octylsulfonylamino group, a 2-ethylhexylsulfonylamino group, and a trifluoromethylsulfonylamino group), carbamoyl groups (such as an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethylhexylaminocarbonyl group, a dodecylaminocarbonyl group, a phenylaminocarbonyl group, a naphthylaminocarbonyl group, and a 2-pyridylaminocarbonyl group), ureido groups (such as a methylureido group, an ethylureido group, a pentylureido group, a cyclohexylureido group, an octylureido group, a dodecylureido group, a phenylureido group, a naphthylureido group, and a 2-pyridylaminoureido group), alkylsulfonyl groups (such as a methylsulfonyl group, an ethylsulfonyl group, a butylsulfonyl group, a cyclohexylsulfonyl group, and a 2-ethylhexylsulfonyl group), arylsulfonyl groups (such as a phenylsulfonyl group, a naphthylsulfonyl group, and a 2-pyridylsulfonyl group), amino groups (such as an amino group, an ethylamino group, a dimethylamino group, a butylamino group, a dibutylamino group, a cyclopentylamino group, a 2-ethylhexylamino group, a dodecylamino group, an anilino group, a naphthylamino group, and a 2-pyridylamino group), alkylsulfonyloxy groups (methanesulfonyloxy group), a cyano group, a nitro group, halogen atoms (such as a fluorine atom, a chlorine atom, and a bromine atom), a hydroxy group, a sulfo group, and a carboxy group.

The number of carbon atoms in the above-described group as the substituent X is not particularly limited, but can be set, for example, in the following range.

The number of carbon atoms in the above-described alkyl group can be in the same range as the number of carbon atoms in the aryl group which can be adopted as R¹ to R⁴, or separately from this, the number thereof can also be 1 to 20 (preferably 1 to 15 and more preferably 1 to 8). The number of carbon atoms in the above-described alkenyl group is preferably 2 to 20, more preferably 2 to 12, and still more preferably 2 to 8. The number of carbon atoms in the above-described alkynyl group is preferably 2 to 40, more preferably 2 to 30, and particularly preferably 2 to 25. Each of the alkyl group, the alkenyl group, and the alkynyl group may be linear, branched, or cyclic, and is preferably linear or branched.

The above-described aryl group includes a group having a single ring or a fused ring, and the number of carbon atoms therein is preferably 6 to 30, more preferably 6 to 20, and still more preferably 6 to 12. The above-described heteroaryl group includes a group formed of a single ring or a fused ring, and a group formed of a single ring or a fused ring having 2 to 8 rings is preferable and a group formed of a single ring or a fused ring having 2 to 4 rings is more preferable. The number of heteroatoms constituting a ring of the heteroaryl group is preferably 1 to 3. Examples of the heteroatoms constituting the ring of the heteroaryl group include a nitrogen atom, an oxygen atom, and a sulfur atom. The heteroaryl group is preferably a group formed of a 5-membered ring or a 6-membered ring. The number of carbon atoms constituting the ring of the heteroaryl group is preferably 3 to 30, more preferably 3 to 18, and still more preferably 3 to 12. The heterocyclic group has the same meaning as the above-described heteroaryl group, except that the heterocyclic group does not have aromaticity.

The alkyl group in the substituent including an alkyl group, such as an alkoxy group, has the same meaning as the above-described alkyl group. In addition, the aryl group or the heteroaryl group in the substituent including an aryl group or a heteroaryl group, such as the aryloxy group and the heteroaryloxy group, has the same meaning as the above-described aryl group or the above-described heteroaryl group.

Among the above, as the substituent X that may be included in the alkyl group and the aryl group, which can be adopted as R¹ to R⁴, an alkyl group, an aryl group, an acyl group, an alkoxy group, an acylamino group, or a sulfonylamino group is preferable.

At least one of R¹, . . . , or R⁴ is an aryl group, and at least one is an alkyl group. The number of aryl groups which can be adopted as R¹ to R⁴ is set to 3 or less, but is preferably 2 or 3 and more preferably 2. On the other hand, the number of alkyl groups which can be adopted as R¹ to R⁴ is set to 3 or less, but is preferably 1 or 2 and more preferably 2. In a case where R¹ to R⁴ have two alkyl groups and two aryl groups, two aspects including an aspect in which R¹ and R² are the aryl groups and an aspect in which R¹ and R³ are the aryl groups are mentioned. In a case where R¹ to R⁴ have a plurality of alkyl groups or aryl groups, the plurality of alkyl groups or aryl groups may be the same or different from each other.

From the viewpoint of easiness of synthesis, an aspect in which R¹ and R³ are aryl groups and R² and R⁴ are alkyl groups is preferable, and an aspect in which R¹ and R³ are the same aryl group and R² and R⁴ are the same alkyl group is most preferable.

R⁵ and R⁶ each independently represent —NR⁹R¹⁰. Here, R⁹ and R¹⁰ are each independently selected from a hydrogen atom, —COR^{N}, —COOR^{N}, —CON(R^{N})₂, or —SO₂R^{N}. In —NR⁹R¹⁰, R⁹ and R¹⁰ bonded to the same nitrogen atom are appropriately selected, but it is preferable that one of R⁹ or R¹⁰ bonded to the same nitrogen atom is a hydrogen atom. As a result, the intramolecular hydrogen bond is formed with an oxygen atom bonded to the carbon four-membered ring, and the compound (1) itself is to be rigid and the light resistance is remarkably improved. The other of R⁹ or R¹⁰ is selected from —COR^{N}, —COOR^{N}, —CON(R^{N})₂, or —SO₂R^{N}, and —COR^{N} or —SO₂R^{N} is preferable. In the compound (1), R⁵ and R⁶ may be —NR⁹R¹⁰'s having different structures, but are preferably —NR⁹R¹⁰ having the same structure.

The R^{N} represents a hydrogen atom, an alkyl group, or an aryl group, and in the compound (1), an alkyl group or an aryl group is preferable, and an alkyl group is more preferable. The alkyl group and the aryl group which can be adopted as R^{N} are not particularly limited, but preferably have the same meaning as the alkyl group and the aryl group, which can be adopted as R¹ to R⁴ described above, respectively. The alkyl group and the aryl group which can be adopted as R^{N} may have a substituent. As such a substituent, a group selected from the above-described substituent X is preferable, and among them, a halogen atom (particularly a fluorine atom), an alkyl group, an alkoxy group, an aryl group, an aryloxy group, an acyl group, or the like is preferable. The halogen-substituted alkyl group may be a group in which a part of hydrogen atoms is substituted or a perhalogenoalkyl group in which all of hydrogen atoms are substituted.

Two R^{N}'s of —CON(R^{N})₂ may be the same or different from each other.

R⁷ and R⁸ each independently represent a substituent. The substituent which can be adopted as R⁷ and R⁸ is not particularly limited, and examples thereof include a group selected from the above-described substituent X. Among them, an alkenyl group, a halogen atom, an alkyl group, an acyl group, an alkoxy group, an acylamino group, a sulfonylamino group, or a hydroxy group is preferable.

The substituent which can be adopted as R⁷ and R⁸ may form a ring. For example, a plurality of R⁷'s or R⁸'s may be bonded to each other to form a fused ring together with the benzene ring. For example, in Exemplary Compound A-15 described later, two ethylene groups bonded to the same benzene ring are bonded to each other to form a benzene ring fused with the benzene ring (that is, a naphthalene ring). The ring formed in this case is not particularly limited, and may be a hydrocarbon ring or a hetero ring and may be an aliphatic ring or an aromatic ring.

The substituent which can be adopted as R⁷ and R⁸ may further have a substituent. Examples of the substituent which may be further included include a group selected from the above-described substituent X.

m and n are each independently an integer of 0 to 3, and are preferably 0 or 1.

In a case where each m and n is 2 or 3, a plurality of R⁷'s or R⁸'s may be the same or different from each other.

In the compound (1), the group represented by each reference numeral in Formula (1) or the substituent in the group represented by each reference numeral has at least one branched alkyl group having 4 or more carbon atoms. The number of carbon atoms in the branched alkyl group is not particularly limited as long as it is 4 or more, but it is preferably in the same range as the number of carbon atoms in the above-described branched alkyl group which can be adopted as R¹ to R⁴.

The total number of branched alkyl groups contained in the compound (1) is not particularly limited, but from the viewpoint of optical properties and solubility, it is preferably 2 or more, more preferably 2 to 6, still more preferably 2 to 4, and even more preferably 2 or 4.

In the compound (1), it is preferable that the branched alkyl group is incorporated as at least one of R¹ to R⁴, R⁷, R⁸, R⁹, or R¹⁰ or as the substituent at the at least one of these, it is more preferably to be incorporated as at least one of R¹ to R⁴, R⁹, or R¹⁰, and it is still more preferably to be incorporated as at least one of R², R⁴, R⁹, or R¹⁰.

In the compound (1), the group represented by each reference numeral in Formula (1) and the like can be applied in combination as appropriate, and it is preferable to apply the groups in combination with preferred groups.

—Squarylium Compound Represented by Formula (2)—

The above-described compound (1) is preferably a squarylium compound represented by Formula (2) (may be referred to as a compound (2)). However, the squarylium compound represented by Formula (2) has at least one branched alkyl group having 4 or more carbon atoms.

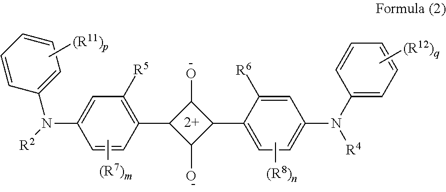

In Formula (2), R² and R⁴ each independently represent an alkyl group. R¹¹ and R¹² represent a substituent, and p and q represent an integer of 0 to 5. R⁵ to R⁸, m, and n have the same meaning as R⁵ to R⁸, m, and n in Formula (1) described above.

The alkyl group which can be adopted as R² and R⁴ has the same meaning as the alkyl group which can be adopted as R¹ to R⁴ in Formula (1).

R¹¹ and R¹² each independently represent a substituent. The substituent which can be adopted as R¹¹ and R¹² has the same meaning as the substituent that may be included in alkyl group and the aryl group, which can be adopted as R¹ to R⁴, and specific examples thereof include a group selected from the above-described substituent X. Among them, an alkyl group, an aryl group, an acyl group, an alkoxy group, an acylamino group, or a sulfonylamino group is preferable.

p and q are each independently an integer of 0 to 5, preferably 0 to 3, more preferably 0 to 2, and still more preferably 1. In a case where each p and q is an integer of 2 or more, a plurality of R¹¹'s or R¹²'s may be the same or different from each other. A position where R¹¹ and R¹² are bonded is not particularly limited, and for example, the position may be an ortho-position (2-position), a meta-position (3-position), or a para-position (4-position) with respect to a ring-constituting carbon atom (1-position) bonded to the nitrogen atom of each benzene ring, and a para-position is preferable.

R⁵ to R⁸, m, and n have the same meaning as R⁵ to R⁸, m, and n in Formula (1), respectively.

In the compound (2), the group represented by each reference numeral in Formula (2) or the substituent in the group represented by each reference numeral has at least one branched alkyl group having 4 or more carbon atoms.

The number of carbon atoms in the branched alkyl group and the total number of branched alkyl groups contained in the compound (2) have the same meaning as the number of carbon atoms described in the compound (1) and the total number of branched alkyl groups contained in the compound (1), respectively.

In the compound (2), it is preferable that the branched alkyl group is incorporated as at least one of R², R⁴, R⁷, R⁸, R⁹, R¹⁰, R¹¹, or R¹² or as the substituent at the at least one of these, it is more preferably to be incorporated as at least one of R², R⁴, R⁹, or R¹⁰, and it is still more preferably to be incorporated as at least one of R² or R⁴ in R², R⁴, R⁹, and R¹⁰.

In the compound (2), the group represented by each reference numeral in Formula (2) and the like can be applied in combination as appropriate, and it is preferable to apply the groups in combination with preferred groups.

Specific examples of the squarylium compound represented by Formula (1) are shown below, but the present invention is not limited thereto. The following specific examples are shown as a tautomer structure of the squarylium compound represented by Formula (1). In addition, in the following specific examples, an alkyl group represented by —CₐH₍₂ₐ₊₁₎ represents a linear alkyl group, and Me represents methyl.

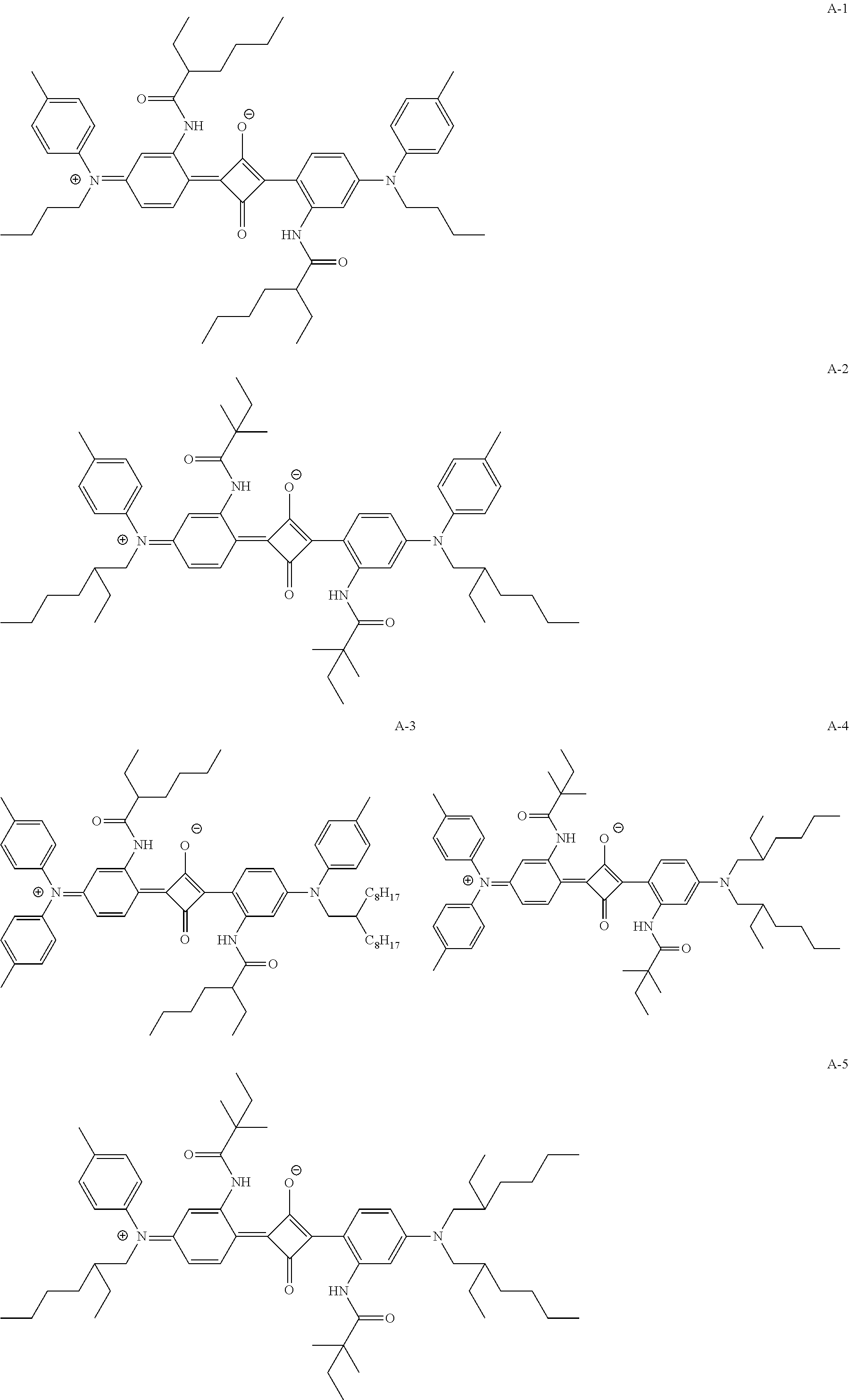

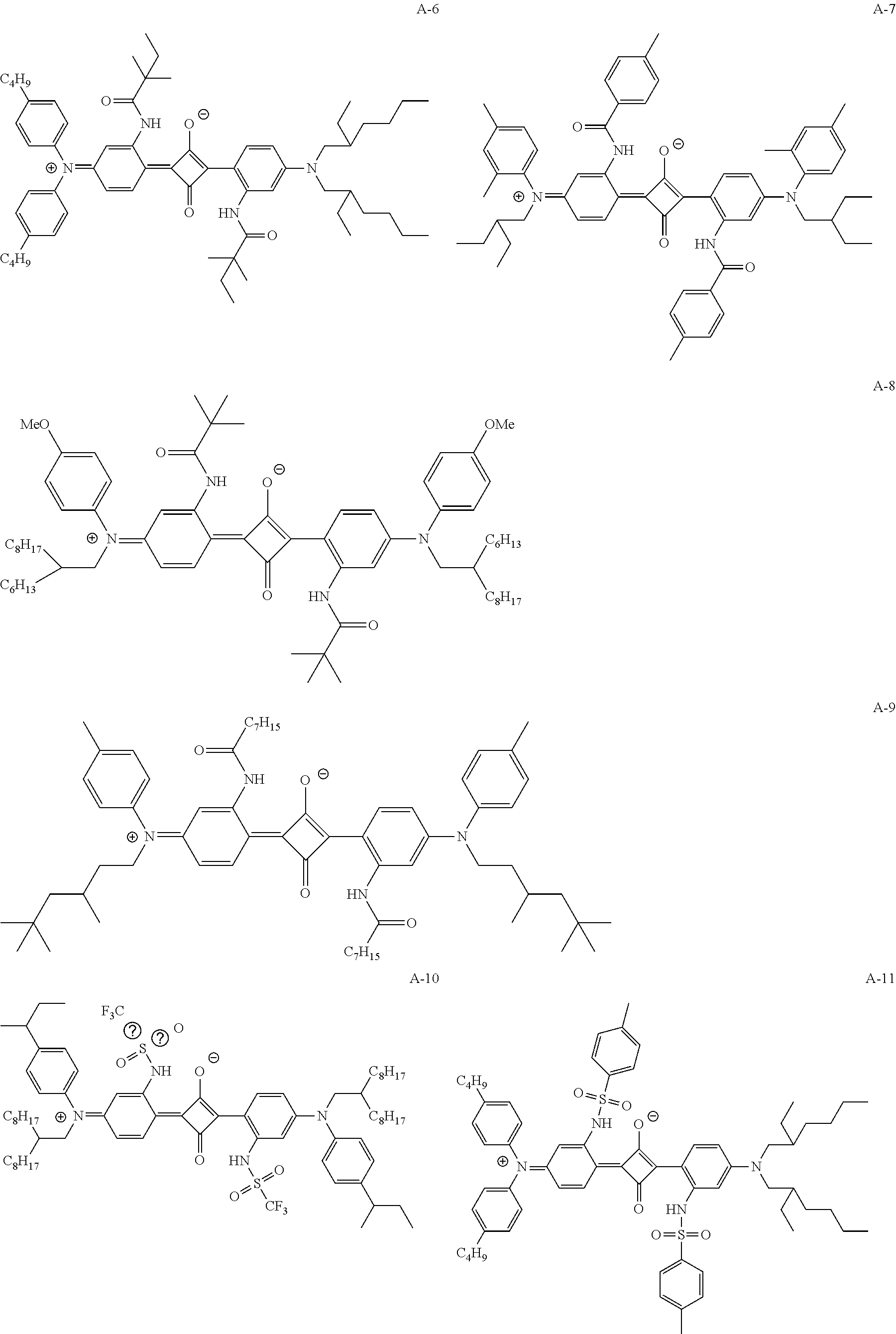

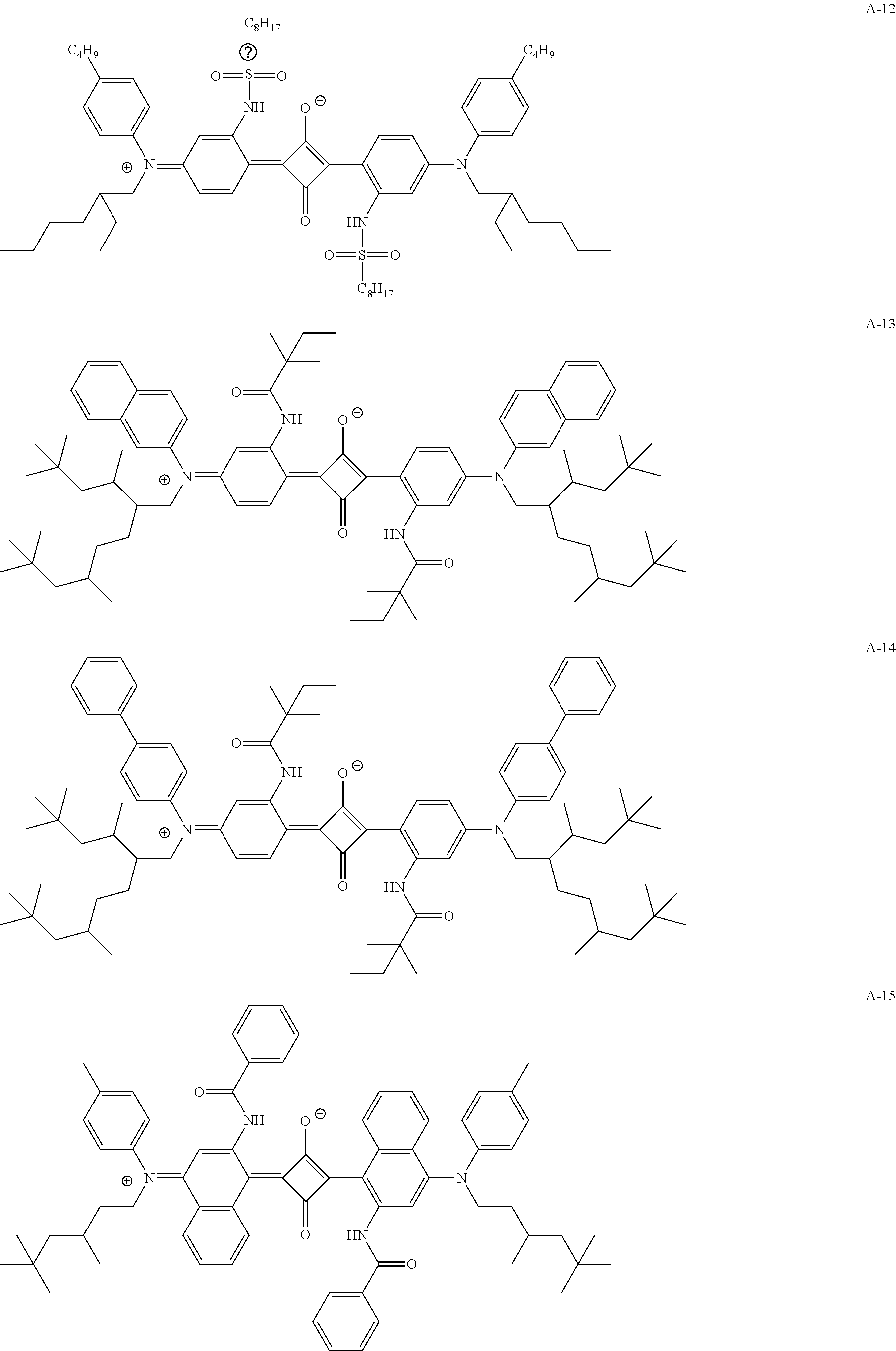

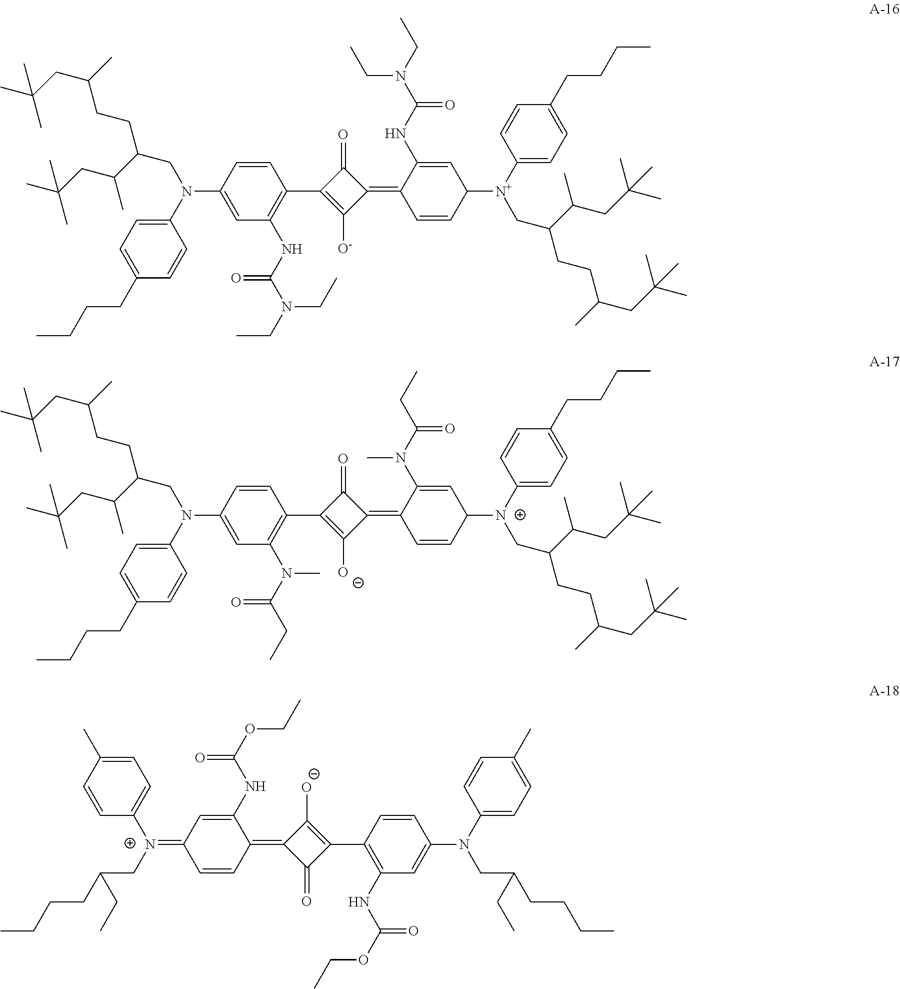

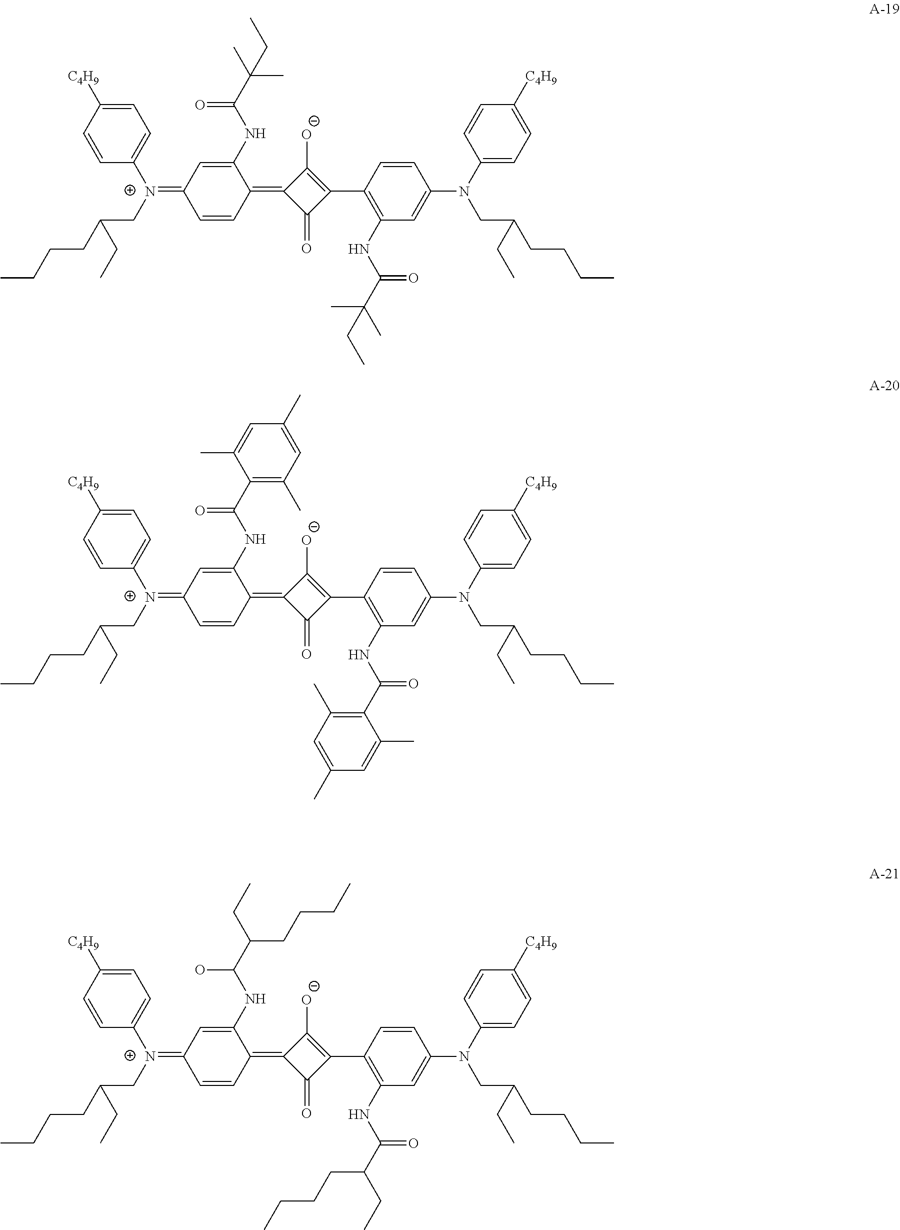

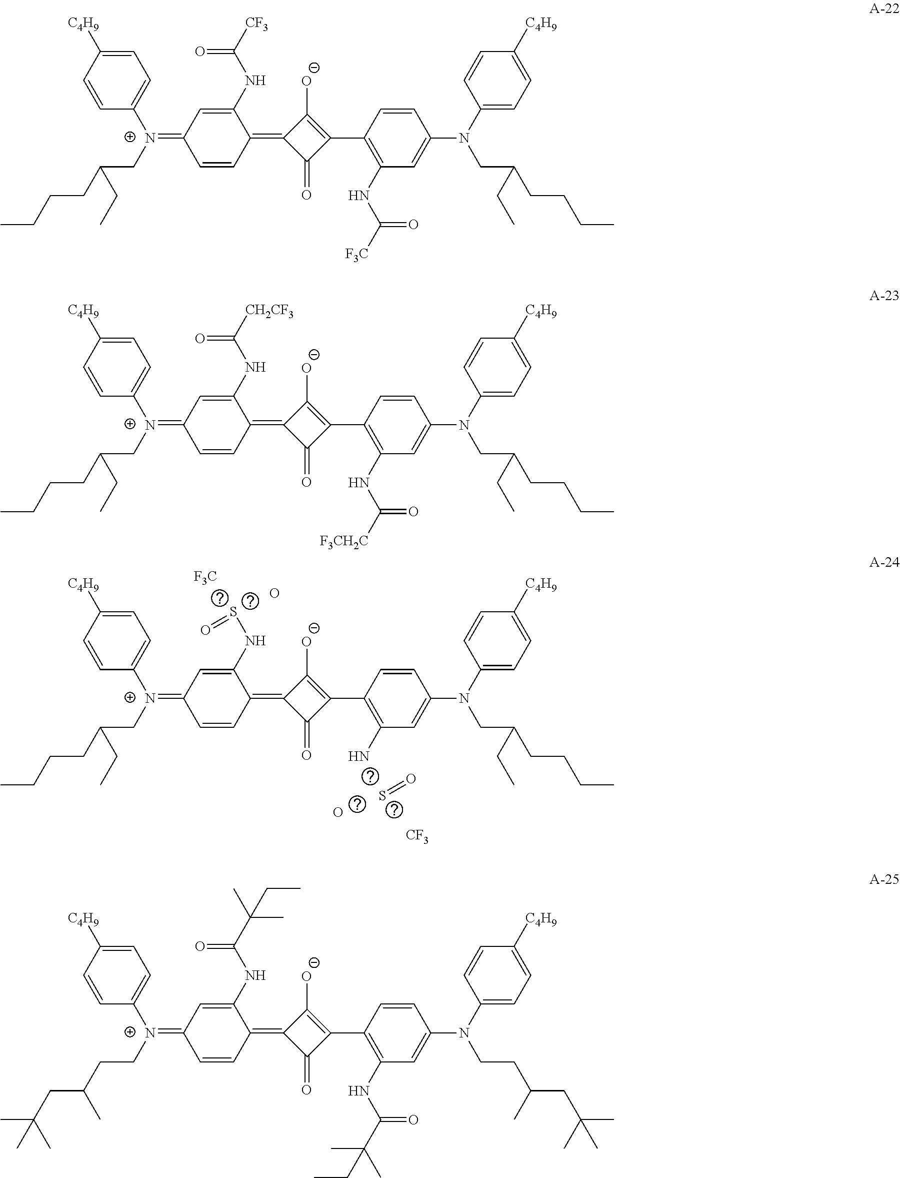

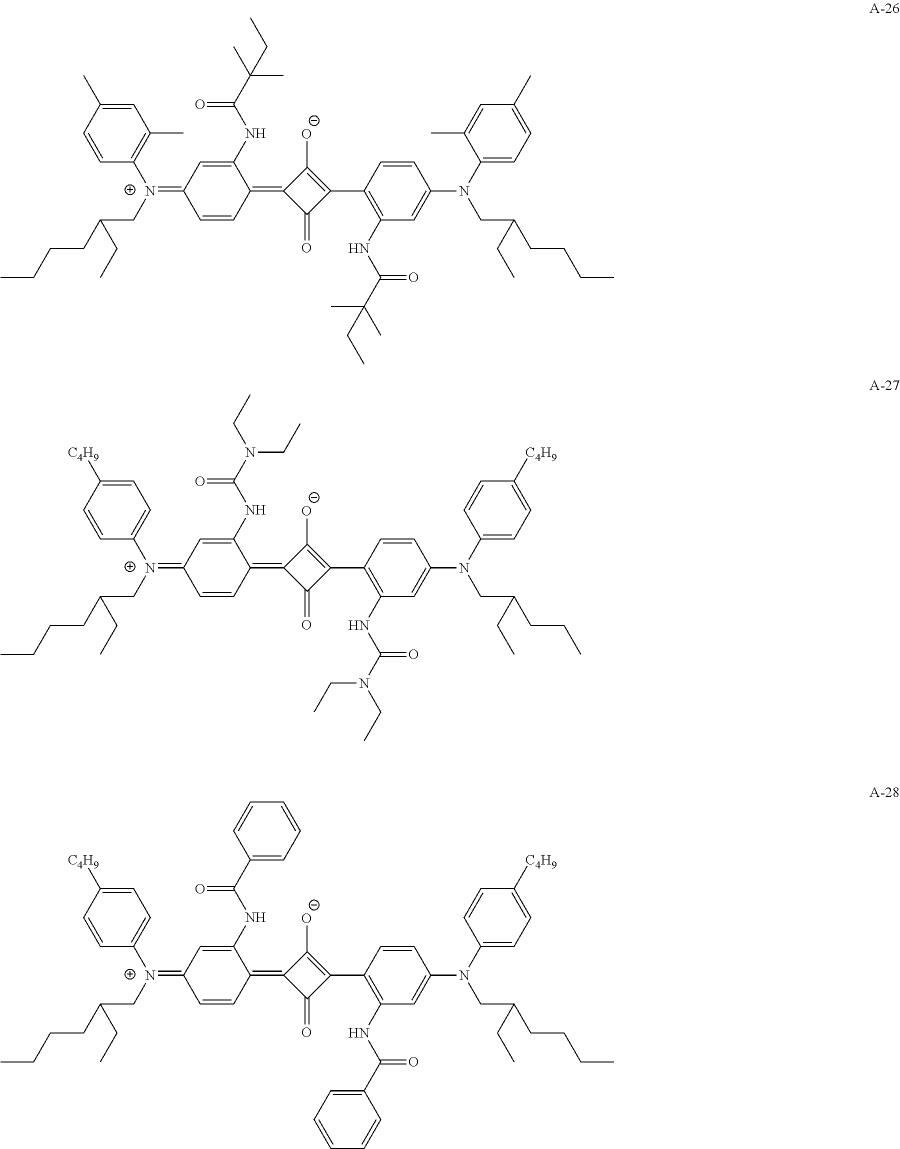

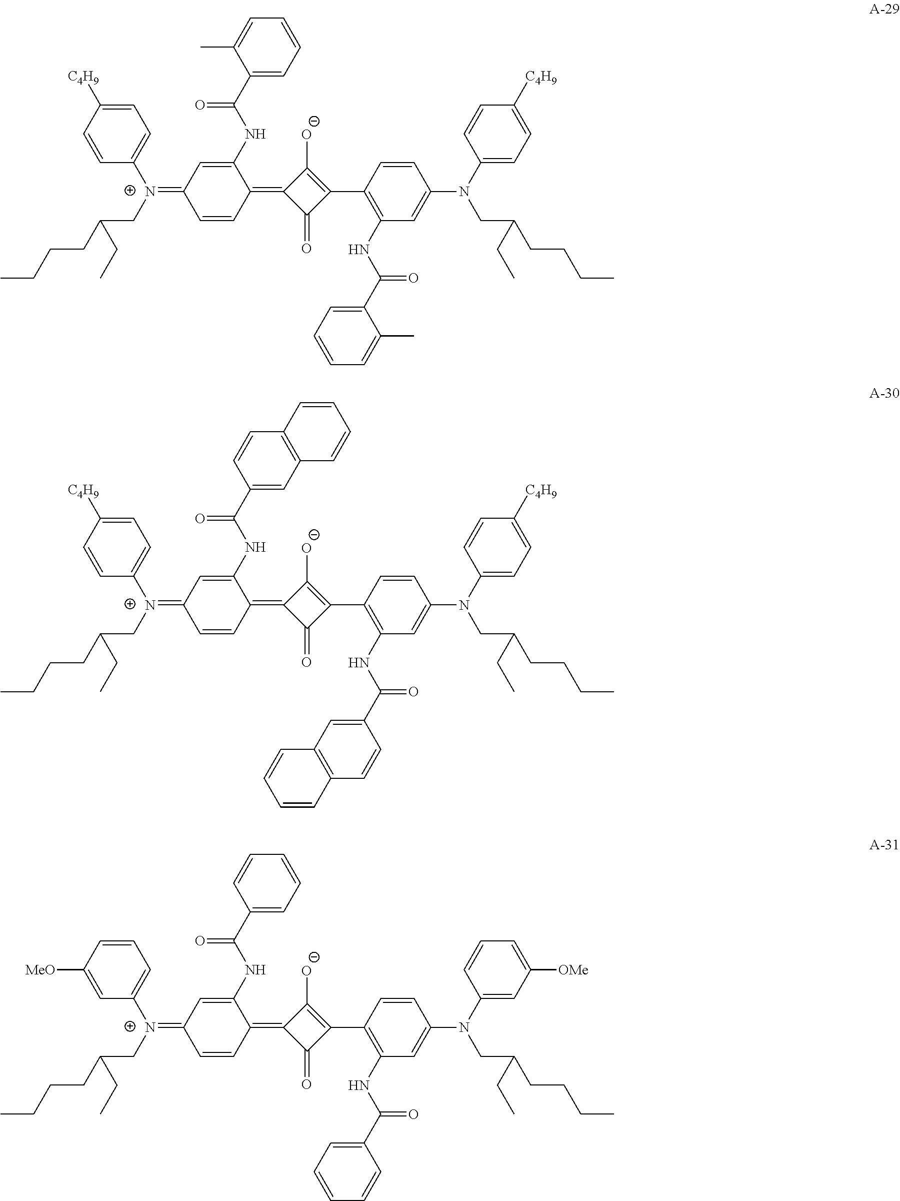

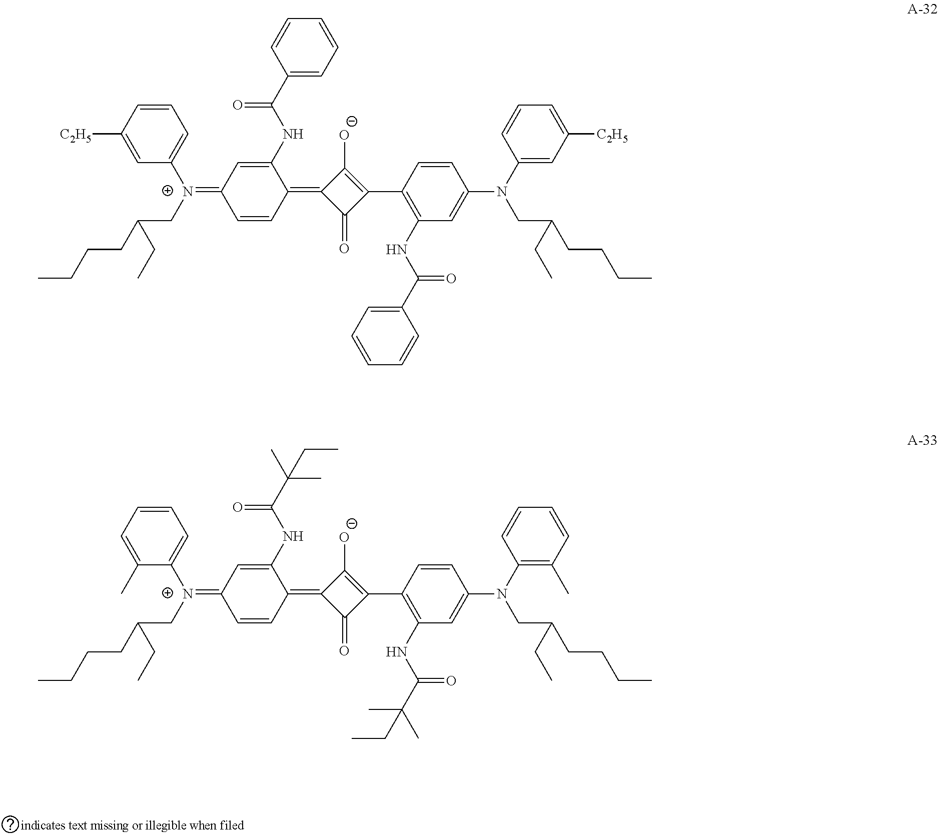

(Squarylium Compound Represented by Formula (3))

Another aspect of the squarylium compound contained in the resin composition according to the embodiment of the present invention is a squarylium compound (3) represented by Formula (3). The compound (3) has at least one group represented by Formula (4M). That is, the compound (3) is a compound in which at least one hydrogen atom of the compound represented by Formula (4) is substituted with a group represented by Formula (4M). In the compound (3), it is preferable that the group represented by each reference numeral in Formula (4) or the substituent in the group represented by each reference numeral has at least one branched alkyl group having 4 or more carbon atoms.

The compound (3) is configured by appropriately selecting the group represented by each reference numeral in the formula from a range described below, but it is preferable to have a symmetrical structure with respect to a carbon four-membered ring in Formula (4) (that a benzene ring included in R⁵ and a benzene ring included in R⁶ have the same chemical structure).

Dye-(Q¹)ₙ₁ Formula (3)

In Formula (3), Dye represents a structural part obtained by removing n1 hydrogen atoms from a squarylium compound (may be referred to as a compound (4)) represented by Formula (4), and Q¹ represents a group represented by Formula (4M). n1 represents an integer of 1 to 6.

—Squarylium compound represented by Formula (4)—

The compound (4) which derives Dye of the compound (3) is represented by Formula (4).

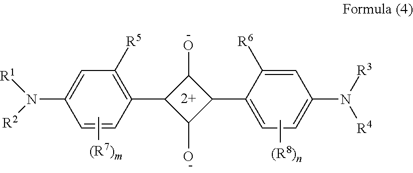

In Formula (4), R¹ to R⁴ represent an alkyl group or an aryl group, which may have a substituent. However, at least one of R¹, . . . , or R⁴ is an aryl group and at least one of R¹, . . . , or R⁴ is an alkyl group. R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent. R⁷ and R⁸ represent a substituent, and m and n represent an integer of 0 to 3.

The compound (4) is the same as the compound (1) described above, except that the compound (4) may not have the branched alkyl group having 4 or more carbon atoms. That is, R¹ to R⁸, m, and n in Formula (4) have the same meaning as R¹ to R⁸, m, and n in Formula (1), respectively.

However, in a case where the group represented by Formula (4M) is introduced into the alkyl group which can be adopted as R¹ to R⁴, the alkyl group is preferably a linear alkyl group, and within the above-described range, the number of carbon atoms thereof is preferably in a range of 1 to 10 and more preferably in a range of 2 to 6.

In addition, in a case where the group represented by Formula (4M) is introduced into —NR⁹R¹⁰, it is preferable that R^{N} included in —COR^{N}, —COOR^{N}, —CON(R^{N})₂, or —SO₂R^{N}, which can be adopted as R⁹ and R¹⁰, is a hydrogen atom or an alkyl group.

The compound (4) may not have the branched alkyl group having 4 or more carbon atoms, but it is preferable that the compound (4) has at least one branched alkyl group having 4 or more carbon atoms. The aspect in which the compound (4) has the branched alkyl group having 4 or more carbon atoms has the same meaning as the aspect in which the compound (1) has the branched alkyl group having 4 or more carbon atoms, and in this case, it is preferable that the compound (4) is the same as the compound (1).

In the compound (4), the group represented by each reference numeral in Formula (4) and the like can be applied in combination as appropriate, and it is preferable to apply the groups in combination with preferred groups.

A portion (atom) in which the hydrogen atom has been removed from the compound (4) serves as a bonding part with L (bonding part represented by “*” the formula) in Formula (4M).

The aspect of removing the hydrogen atom from the compound (4) is not particularly limited, and an appropriate hydrogen atom can be removed. Examples thereof include a hydrogen atom contained in each group represented by any one of R¹, . . . , or R⁸ and a hydrogen atom contained in the benzene ring to which R⁵ or R⁶ is bonded, and a hydrogen atom contained in each group represented by any one of R¹, . . . , or R⁶ is preferable.

The number of hydrogen atoms removed is not particularly limited, and has the same meaning as n1 described later.

The aspect of removing hydrogen atoms from the compound (4) is not particularly limited, and preferred examples thereof include an aspect in which one hydrogen atom is removed from each of the groups represented by R¹ and R², an aspect in which one hydrogen atom is removed from each of the groups represented by R¹ and R³ or each of the groups represented by R² and R⁴, an aspect in which one hydrogen atom is removed from each of the groups represented by R⁵ and R⁶ (preferably a group other than a hydrogen atom), and a combination of these aspects; and more preferred examples thereof include an aspect in which one hydrogen atom is removed from each of the groups represented by R¹ and R³ or each of the groups represented by R² and R⁴, an aspect in which one hydrogen atom is removed from each of the groups represented by R⁵ and R⁶, and a combination of these aspects. From the viewpoint of solubility, it is preferable to be an aspect in which one hydrogen atom is removed from each group represented by R⁵ and R⁶.

—Squarylium compound represented by Formula (5)—

The above-described compound (4) is preferably a squarylium compound represented by Formula (5) (may be referred to as a compound (5)).

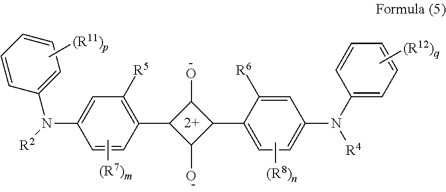

In Formula (5), R² and R⁴ each independently represent an alkyl group. R¹¹ and R¹² represent a substituent, and p and q represent an integer of 0 to 5. R⁵ to R⁸, m, and n have the same meaning as R⁵ to R⁸, m, and n in Formula (4).

The alkyl group which can be adopted as R² and R⁴ has the same meaning as the alkyl group which can be adopted as R¹ to R⁴ in Formula (1).

R¹¹ and R¹² each independently represent a substituent. The substituent which can be adopted as R¹¹ and R¹² has the same meaning as the substituent that may be included in alkyl group and the aryl group, which can be adopted as R¹ to R⁴, and specific examples thereof include a group selected from the above-described substituent X. Among them, an alkyl group, an aryl group, an acyl group, an alkoxy group, an acylamino group, or a sulfonylamino group is preferable.

p and q are each independently an integer of 0 to 5, preferably 0 to 3, more preferably 0 to 2, and still more preferably 1. In a case where each p and q is an integer of 2 or more, a plurality of R¹¹'s or R¹²'s may be the same or different from each other. A position where R¹¹ and R¹² are bonded is not particularly limited, and for example, the position may be a meta-position (3-position) or a para-position (4-position) with respect to a ring-constituting carbon atom (1-position) bonded to the nitrogen atom of each benzene ring, and a para-position is preferable.

R⁵ to R⁸, m, and n have the same meaning as R⁵ to R⁸, m, and n in Formula (4), respectively.

The compound (5) is a preferred aspect of the compound (4), but it can be said that the compound (5) is the same as the compound (2) described above, except that the compound (5) may not have the branched alkyl group having 4 or more carbon atoms. However, it is preferable that the compound (5) has at least one branched alkyl group having 4 or more carbon atoms. The aspect in which the compound (5) has the branched alkyl group having 4 or more carbon atoms has the same meaning as the aspect in which the compound (4) has the branched alkyl group having 4 or more carbon atoms, and in this case, it is preferable that the compound (5) is the same as the compound (2).

In the compound (5), the group represented by each reference numeral in Formula (5) and the like can be applied in combination as appropriate, and it is preferable to apply the groups in combination with preferred groups.

The aspect of removing the hydrogen atom from the compound (5) is not particularly limited, and an appropriate hydrogen atom can be removed. Examples thereof include a hydrogen atom contained in each group represented by any one of R², R⁴ to R⁸, R¹¹, or R¹² and a hydrogen atom contained in the benzene ring to which R⁵ or R⁶, or R¹¹ or R¹² is bonded. The number of hydrogen atoms removed is not particularly limited, and has the same meaning as n1 described later.

The aspect of removing hydrogen atoms from the compound (5) is not particularly limited, and preferred examples thereof include an aspect in which one hydrogen atom is removed from each of the groups represented by R² and R⁴, an aspect in which one hydrogen atom is removed from each of the groups represented by R⁵ and R⁶ (preferably a group other than a hydrogen atom), and a combination of these aspects.

In Formula (3), n1 represents the number of Q1's bonded to Dye, and usually is appropriately selected from a range of 1 to the number of hydrogen atoms in the compound (4). For example, n1 can be an integer of 1 to 6, preferably an integer of 1 to 4 and more preferably 1 or 2. In a case where n1 is an integer of 2 or more, a plurality of Q¹'s may be the same or different from each other.

Q¹ in Formula (3) represents a group represented by Formula (4M).

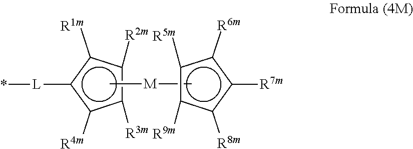

In Formula (4M), L represents a single bond or a divalent linking group which is not conjugated with Dye. R^{1m} to R^{9m} represent a hydrogen atom or a substituent. M represents Fe, Co, Ni, Ti, Cu, Zn, Zr, Cr, Mo, Os, Mn, Ru, Sn, Pd, Rh, V, or Pt. * represents a bonding part with Dye.

In the compound (3), in a case where the above-described group represented by Formula (4M) is introduced into each group represented by any one of R¹, . . . , or R⁸ in Formula (4), L in Formula (4M) is interpreted as a single bond.

In the compound (3), in a case where L in Formula (4M) is a divalent linking group, Dye is a structure up to a portion (atom) where a conjugated structure is interrupted by linking with L. That is, in a case where L is not a single bond but a divalent linking group, the bonding portion of L with Dye does not have a conjugated structure. In other words, L is a single bond in a case where a conjugated structure continues from Dye to the group represented by Formula (4M) (metallocene structural part) (that is, a case where a conjugated structure continues from Dye to the metallocene skeleton in Formula (4M)). Here, the conjugated structure means a structure forming a system of connected p-orbitals having delocalized electrons located in alternating single bond and multiple bond, and also includes a structure including a p-orbital donating group, a p-orbital donating atom, or a p-orbital donating group and a p-orbital donating atom. Examples of the p-orbital donating group include a carbonyl group and a sulfonyl group. The p-orbital donating atom is an atom having two lone electron-pairs, one of which occupies a p-orbital, and examples of an atom which can be the p-orbital donating atom include an oxygen atom, a nitrogen atom, and a sulfur atom. In a case of including a p-orbital donating group and a p-orbital donating atom, examples thereof include a structure of a combination of a plurality (preferably an integer number of 2 to 10) of the p-orbital donating atom and the p-orbital donating group. For example, a divalent group represented by —O—CO—, —NH—CO—, —NH—SO₂—, —NH—CO—NH—, and the like is a group which forms the conjugated structure. In the present invention, in a case where L in Formula (4M) is a single bond, a cyclopentadienyl ring (ring having R^{1m} in Formula (4M)) directly bonded to Dye is not included in the conjugated structure conjugated with Dye.

In view of the above description, the divalent linking group which can be adopted as L is not particularly limited as long as a linking group which is not conjugated with Dye, and the above-described conjugated structure may be included inside the divalent linking group or in an end portion of a cyclopentadiene ring in Formula (4M). Examples of the divalent linking group include an alkylene group having 1 to 20 carbon atoms, an arylene group having 6 to 20 carbon atoms, a divalent heterocyclic group obtained by removing two hydrogen atoms from a hetero ring, —CH═CH—, —CO—, —CS—, —NR— (R represents a hydrogen atom or a monovalent substituent), —O—, —S—, —SO₂—, —N═CH—, and a linking group, among divalent linking groups formed of a combination of a plurality (preferably 2 to 6) of groups selected from the group consisting thereof, which is not conjugated with Dye. A group selected from the group consisting of an alkylene group having 1 to 8 carbon atoms, an arylene group having 6 to 12 carbon atoms, —CH═CH—, —CO—, —NR— (R is as defined above), —O—, —S—, —SO₂—, and —N═CH—, or a linking group, among divalent linking groups formed of a combination of two or more (preferably 2 to 6) groups selected from the group consisting thereof, which is not conjugated with Dye is preferable, and a group selected from the group consisting of an alkylene group having 1 to 4 carbon atoms, a phenylene group, —CO—, —NH—, —O—, and —SO₂—, or a linking group, among divalent linking groups formed of a combination of two or more (preferably 2 to 6) groups selected from the group consisting thereof, which is not conjugated with Dye is particularly preferable. The divalent linking group of a combination is not particularly limited, but is preferably a group including —CO—, —NH—, —O—, or —SO₂—. Examples thereof include a linking group, among linking groups including a group formed of a combination of two or more of —CO—, —NH—, —O—, and —SO₂— and linking groups formed of at least one of —CO—, —NH—, —O—, or —SO₂—, and an alkylene group or an arylene group, which is not conjugated with Dye. Examples of the linking group including a group formed of a combination of two or more of —CO—, —NH—, —O—, and —SO₂— include a linking group, among linking groups including —COO—, —OCO—, —CONH—, —NHCOO—, —NHCONH—, or —SO₂NH—, which is not conjugated with Dye. Examples of the linking group formed of at least one of —CO—, —NH—, —O—, or —SO₂—, and an alkylene group or an arylene group include a linking group, among groups of a combination of —CO—, —COO—, or —CONH—, and an alkylene group or an arylene group, which is not conjugated with Dye.

The substituent which can be adopted as R is not particularly limited, and examples thereof include the above-described substituent X.

L is preferably a single bond, a group selected from the group consisting of an alkylene group having 1 to 8 carbon atoms, an arylene group having 6 to 12 carbon atoms, —CH═CH—, —CO—, —NR— (R is as defined above), —O—, —S—, —SO₂—, and —N═CH—, or a group of a combination of two or more groups selected from the group consisting thereof.

L may have one or a plurality of substituents. The substituent which may be included in L is not particularly limited, and has the same meaning, for example, as the above-mentioned substituent X. In a case where L has a plurality of substituents, substituents bonded to an adjacent atom may be bonded to each other to further form a ring structure.

The alkylene group which can be adopted as L may be linear, branched, or cyclic, as long as the alkylene group has carbon atoms in a range of 1 to 20. Examples thereof include methylene, ethylene, propylene, methylethylene, methylmethylene, dimethylmethylene, 1,1-dimethylethylene, butylene, 1-methylpropylene, 2-methylpropylene, 1,2-dimethylpropylene, 1,3-dimethylpropylene, 1-methylbutylene, 2-methylbutylene, 3-methylbutylene, 4-methylbutylene, 2,4-dimethylbutylene, 1,3-dimethylbutylene, pentylene, hexylene, heptylene, octylene, ethane-1,1-diyl, propane-2,2-diyl, cyclopropane-1,1-diyl, cyclopropane-1,2-diyl, cyclobutane-1,1-diyl, cyclobutane-1,2-diyl, cyclopentane-1,1-diyl, cyclopentane-1,2-diyl, cyclopentane-1,3-diyl, cyclohexane-1,1-diyl, cyclohexane-1,2-diyl, cyclohexane-1,3-diyl, cyclohexane-1,4-diyl, and methylcyclohexane-1,4-diyl.

In a case where a linking group including at least one of —CO—, —CS—, —NR— (R is as defined above), —O—, —S—, —SO₂—, or —N═CH— is adopted in an alkylene group as L, the group of —CO— and the like may be incorporated at any position in the alkylene group, and the number to be incorporated is not particularly limited.

The arylene group which can be adopted as L is not particularly limited as long as it is a group derived by removing a hydrogen atom from an aryl group having 6 to 20 carbon atoms.

The heterocyclic group which can be adopted as L is not particularly limited, and examples thereof include a group including an aliphatic heterocyclic ring or an aromatic heterocyclic ring. As the heterocyclic group, a group of a 5-membered ring or a 6-membered ring is preferable. Examples of the heterocyclic group which can be adopted as L include a group obtained by removing two hydrogen atoms from each ring of a pyrrole ring, a furan ring, a thiophene ring, an imidazole ring, a pyrazole ring, a thiazole ring, an oxazole ring, a triazole ring, an indole ring, an indolenine ring, an indoline ring, a pyridine ring, a pyrimidine ring, a quinoline ring, a benzothiazole ring, a benzoxazole ring, or a pyrazolotriazole ring.

In Formula (4M), the remaining partial structure excluding the linking group L corresponds to a structure (metallocene structural part) in which one hydrogen atom is removed from a metallocene compound. In the present invention, a known metallocene compound can be used, without particular limitation, as the metallocene compound which is the metallocene structural part, as long as a compound (compound having a hydrogen atom bonded in place of L) accommodate with the partial structure defined by Formula (4M). Hereinafter, the metallocene structural part defined by Formula (4M) will be specifically described.

In Formula (4M), R^{1m} to R^{9m} each represent a hydrogen atom or a substituent. The substituent which can be adopted as R^{1m} to R^{9m} is not particularly limited, and can be selected from, for example, the substituent X. Each of R^{1m} to R^{9m} is preferably a hydrogen atom, a halogen atom, an alkyl group, an acyl group, an alkoxy group, an amino group, or an amide group, more preferably a hydrogen atom, a halogen atom, an alkyl group, an acyl group, or an alkoxy group, still more preferably a hydrogen atom, a halogen atom, an alkyl group, or an acyl group, particularly preferably a hydrogen atom, a halogen atom, or an alkyl group, and most preferably a hydrogen atom.

Among the alkyl groups which can be adopted as R¹, the alkyl group which can be adopted as R^{1m} to R^{9m} is preferably an alkyl group having 1 to 8 carbon atoms, and examples thereof include methyl, ethyl, propyl, isopropyl, butyl, sec-butyl, tert-butyl, isobutyl, pentyl, tert-pentyl, hexyl, octyl, and 2-ethylhexyl.

The alkyl group may have a halogen atom as a substituent. Examples of the alkyl group substituted with a halogen atom include chloromethyl, dichloromethyl, trichloromethyl, bromomethyl, dibromomethyl, tribromomethyl, fluoromethyl, difluoromethyl, trifluoromethyl, 2,2,2-trifluoroethyl, perfluoroethyl, perfluoropropyl, and perfluorobutyl.

In addition, in the alkyl group which can be adopted as Rim and the like, at least one methylene group forming a carbon chain may be substituted with —O— or —CO—. Examples of the alkyl group in which a methylene group is substituted with —O— include an alkyl group in which a terminal methylene group is substituted, such as methoxy, ethoxy, propoxy, isopropoxy, butoxy, secondary butoxy, tertiary butoxy, 2-methoxyethoxy, chloromethyloxy, dichloromethyloxy, trichloromethyloxy, bromomethyloxy, dibromomethyloxy, tribromomethyloxy, fluoromethyloxy, difluoromethyloxy, trifluoromethyloxy, 2,2,2-trifluoroethyloxy, perfluoroethyloxy, perfluoropropyloxy, and perfluorobutyloxy, and an alkyl group in which an internal methylene group of a carbon chain is substituted, such as 2-methoxyethyl. Examples of the alkyl group in which a methylene group is substituted with —CO— include acetyl, propionyl, monochloroacetyl, dichloroacetyl, trichloroacetyl, trifluoroacetyl, propan-2-one-1-yl, and butan-2-one-1-yl.

In Formula (4M), M is an atom capable of forming the metallocene compound, and represents Fe, Co, Ni, Ti, Cu, Zn, Zr, Cr, Mo, Os, Mn, Ru, Sn, Pd, Rh, V, or Pt. Among these, M is preferably Fe, Ti, Co, Ni, Zr, Ru, or Os, more preferably Fe, Ti, Ni, Ru, or Os, still more preferably Fe or Ti, and most preferably Fe.

The group represented by Formula (4M) is preferably a group of a combination of preferred L, R^{1m} to R^{9m}, and M, and examples thereof include a group of a combination of, as L, a single bond, a group selected from the group consisting of an alkylene group having 2 to 8 carbon atoms, an arylene group having 6 to 12 carbon atoms, —CH═CH—, —CO—, —NR— (R is as defined above), —O—, —S—, —SO₂—, and —N═CH—, or a group of a combination of two or more groups selected from the group consisting thereof, as R^{1m} to R^{9m}, a hydrogen atom, a halogen atom, an alkyl group, an acyl group, or an alkoxy group, and as M, Fe.

Specific examples of the squarylium compound represented by Formula (3) are shown below, but the present invention is not limited thereto. The following specific examples are shown as a tautomer structure of the squarylium compound represented by Formula (3). In the following specific examples, an alkyl group represented by —CₐH₍₂ₐ₊₁₎ represents a linear alkyl group, and Me represents methyl.

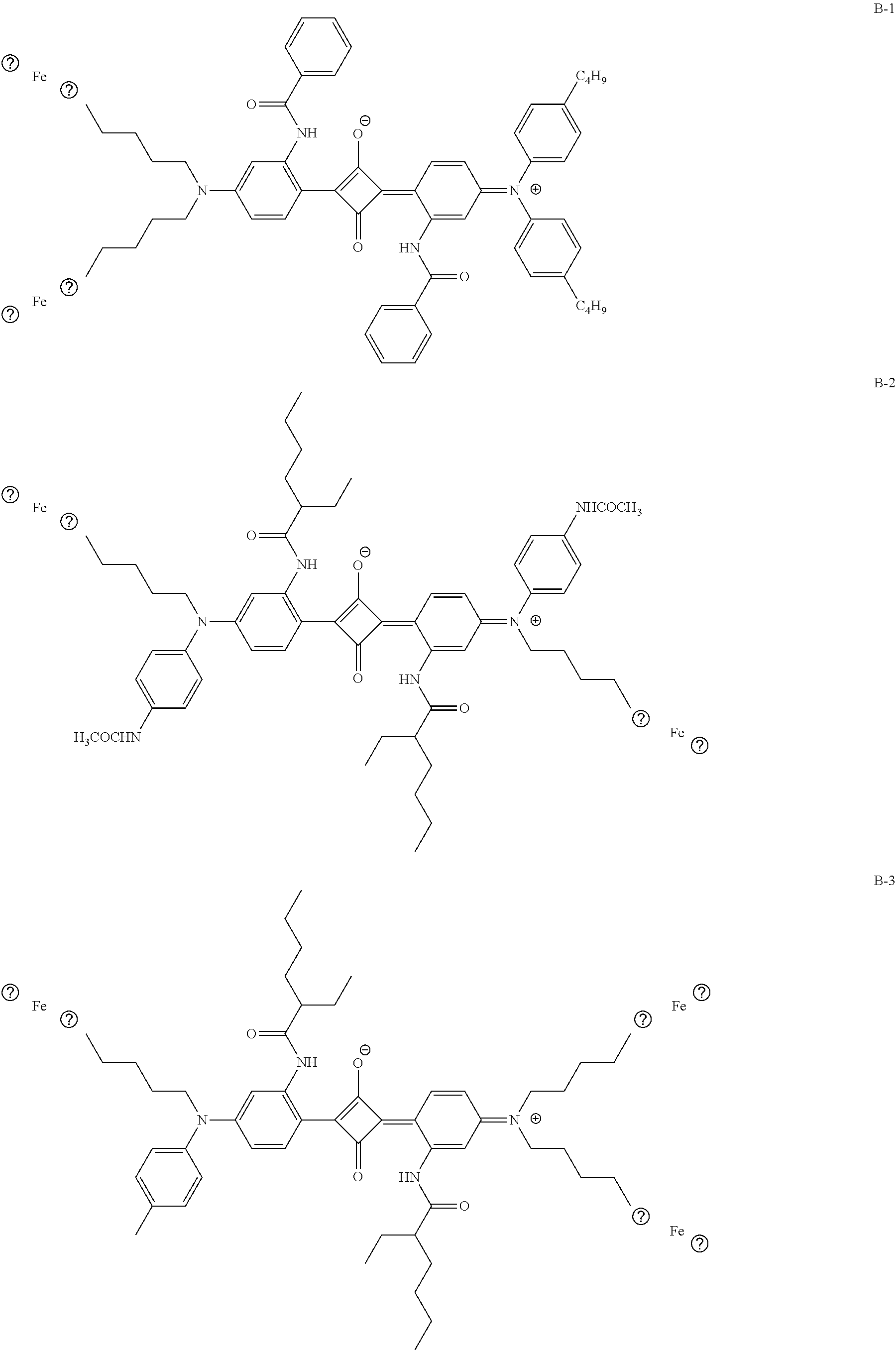

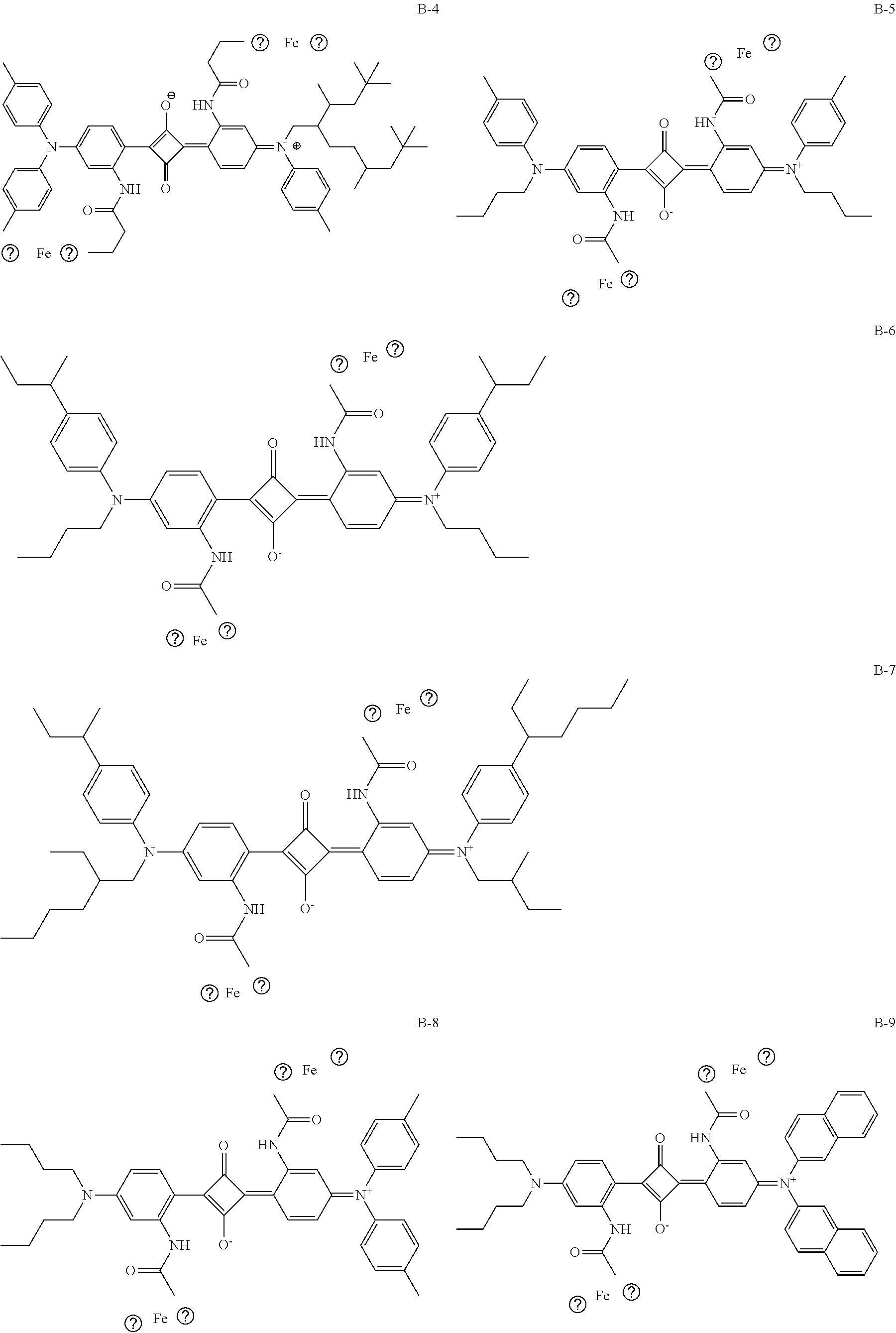

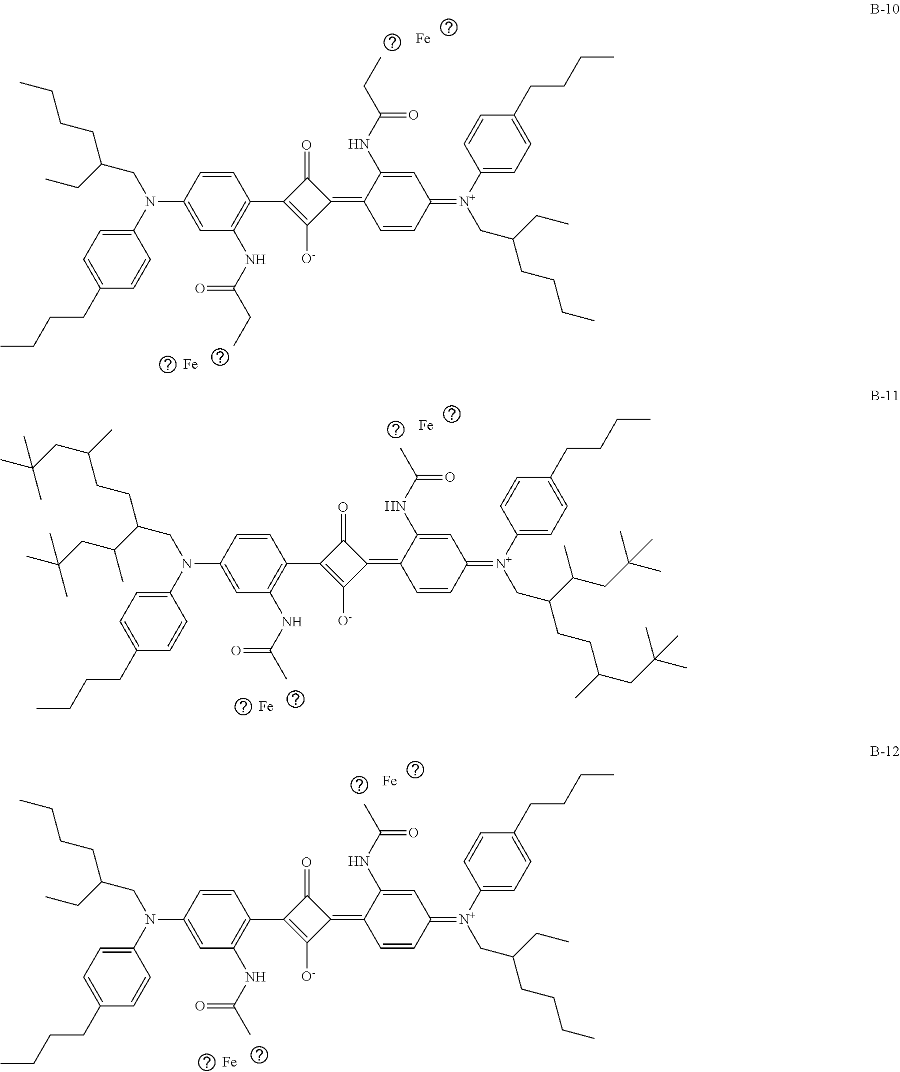

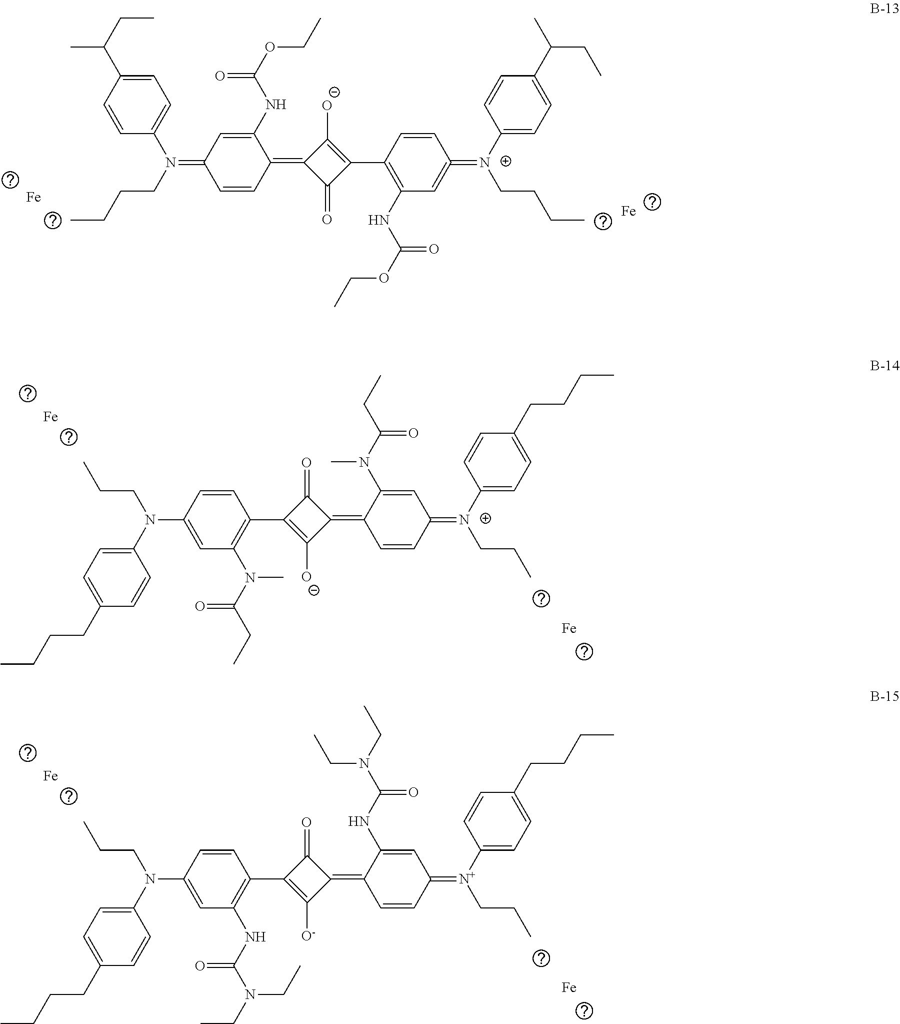

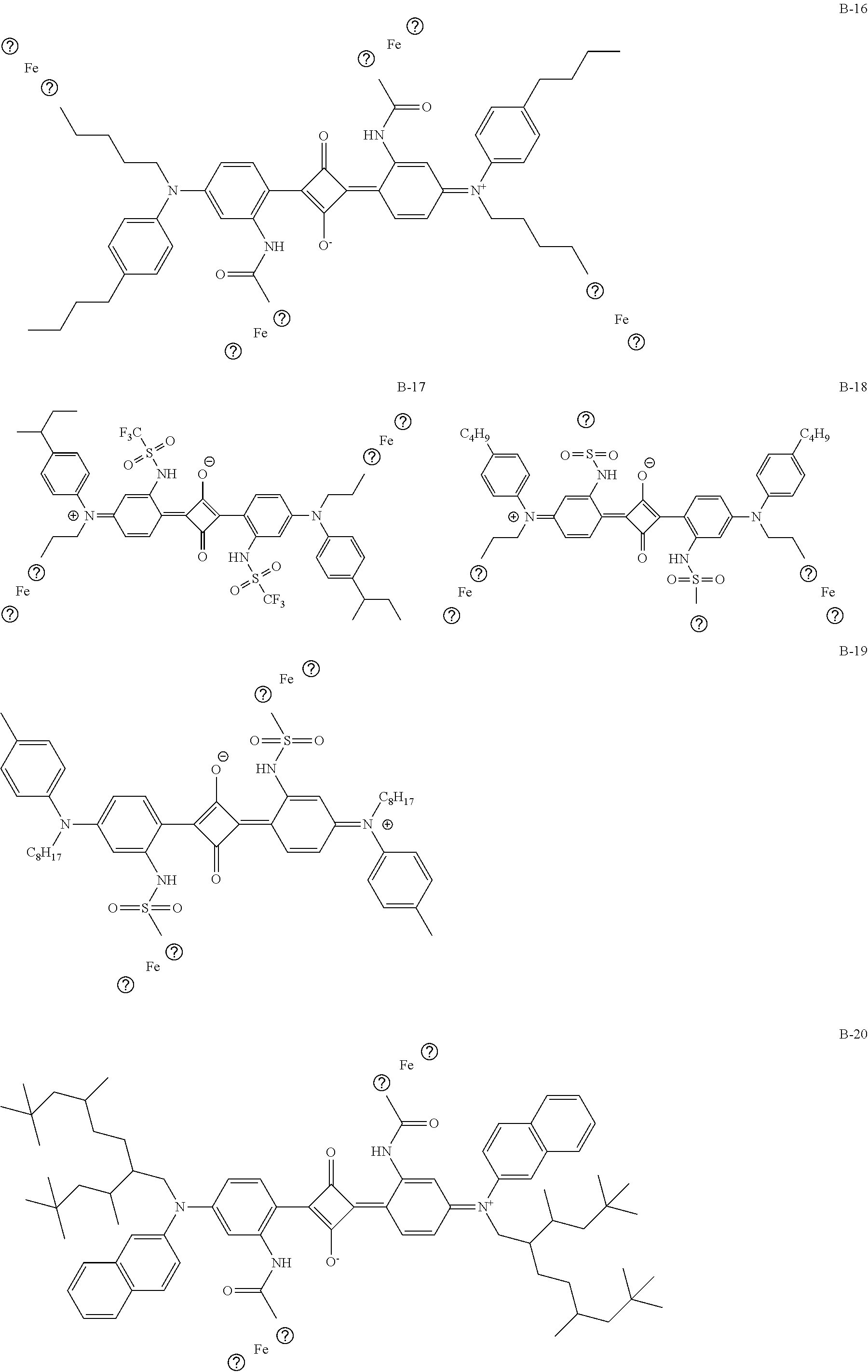

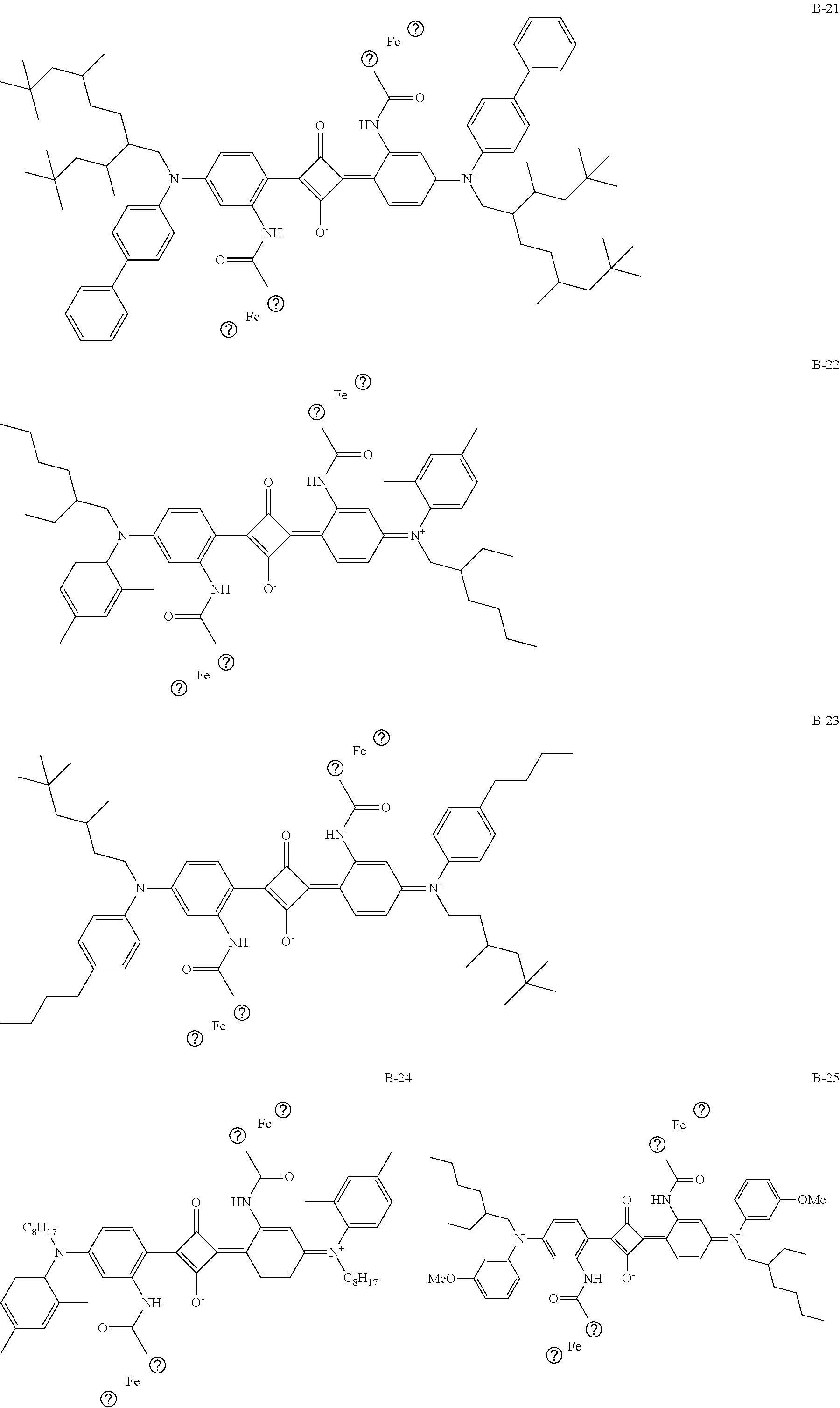

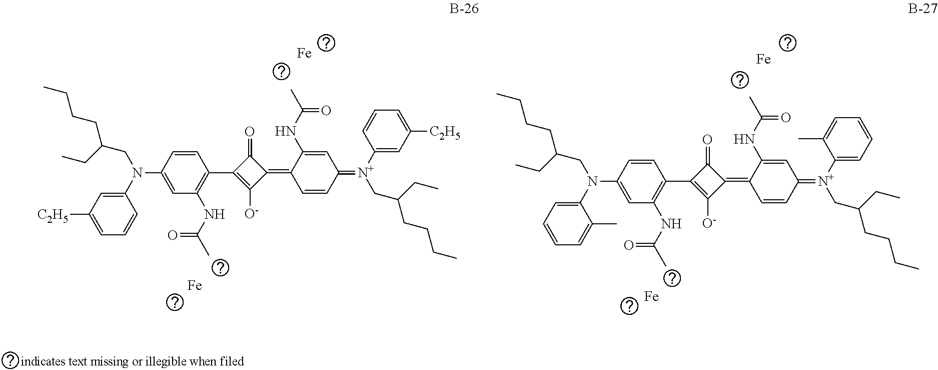

A content of the squarylium compound in the resin composition according to the embodiment of the present invention is not particularly limited, and is appropriately set in consideration of the type or solubility of the squarylium compound, required optical properties, and the like. For example, the above-described content is preferably 0.005 to 15 parts by mass, more preferably 0.01 to 10 parts by mass, and still more preferably 0.01 to 5 parts by mass with respect to 100 parts by mass of a binder resin described later. In the resin composition according to the embodiment of the present invention, the squarylium compound can be set to be a high content, and in this case, for example, the content can be set to 10 to 30 parts by mass.

In addition, the squarylium compound is easily dissolved in a solvent, and for example, in “Evaluation of solubility” in Examples described later, the squarylium compound has a solubility of 0.01 parts by mass or more in 100 parts by mass of a mixed solvent of toluene/cyclohexanone.

In a case where an optical filter contains two or more kinds of the squarylium compounds, the above-described content is the total content thereof.

In a case where the optical filter according to the embodiment of the present invention also serves as a polarizing plate-protective film or a pressure-sensitive adhesive layer, which will be described later, it is sufficient that the content of the coloring agent (squarylium compound) is within the above-described range.

(Synthesis Method of Squarylium Compound)

The squarylium compound represented by each formula can be synthesized according to a known method. For example, the synthesis can be carried out according to the synthesis methods disclosed in JP1985-169453A (JP-S60-169453A), JP2009-036811A, and WO2019/167930A1, a synthesis method described in Examples later, and the like.

Examples of a preferred method for synthesizing (producing) the squarylium compound represented by Formula (1) include a method of synthesizing the squarylium compound represented by Formula (1) by reacting a compound represented by Formula (A) with squaric acid or a compound represented by Formula (B) (hereinafter, may be referred to as a preferred producing method). In the following formula, the compound to be reacted with the squaric acid is the compound represented by Formula (A), but it is synonymous with a combination of the compound represented by Formula (A) and a compound represented by Formula (B1) described later.

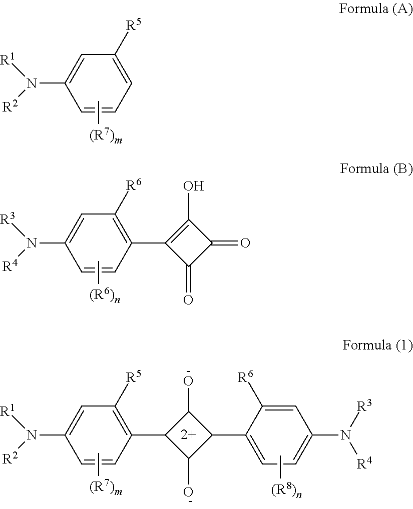

In Formula (A), Formula (B), and Formula (1), R¹ to R⁴ represent an alkyl group or an aryl group, which may have a substituent. R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent. R⁷ and R⁸ represent a substituent, and m and n represent an integer of 0 to 3. Each reference numeral in Formula (A), Formula (B), and Formula (1) is the same as the corresponding reference numeral in Formula (1) described above.

However, in a case where the compound represented by Formula (A) is reacted with the squaric acid, as a combination of the compounds represented by Formula (A) to be reacted with the squaric acid, at least one of R¹ or R² is an aryl group, at least one of R¹ or R² is an alkyl group, and at least one of the compounds represented by Formula (A) has at least one branched alkyl group having 4 or more carbon atoms. It is preferable that two molecules of the compound represented by Formula (A) to be reacted with the squaric acid have the same chemical structure. In the combination the compound represented by Formula (A) and a compound represented by Formula (B1) described later, “R¹ or R²” described above is read as “R¹, . . . , or R⁴”, and “at least one of the compounds represented by Formula (A)” is read as “at least one of the compound represented by Formula (A) or the compound represented by Formula (B1)”.

In a case where the compound represented by Formula (A) is reacted with the compound represented by Formula (B), as a combination of the compounds represented by Formula (A) or Formula (B), which are reacted with each other, at least one of R¹, . . . , or R⁴ is an aryl group, at least one of R¹, . . . , or R⁴ is an alkyl group, and at least one of the compound represented by Formula (A) or the compound represented by Formula (B) has at least one branched alkyl group having 4 or more carbon atoms. It is preferable that the compound represented by Formula (A) has a chemical structure different from that of an aminobenzene moiety in Formula (B).

The squarylium compound represented by Formula (1) has at least one branched alkyl group having 4 or more carbon atoms.

In the above-described combinations, the aspect of having the aryl group and the alkyl group and the aspect of further having at least one branched alkyl group having 4 or more carbon atoms are the same as each aspect of the compound represented by Formula (1) described above.

In the above-described preferred producing method, a compound to be reacted with the compound represented by Formula (A) can be selected depending on the chemical structure of the squarylium compound to be produced. For example, in a case where the squarylium compound represented by Formula (1) has a symmetric chemical structure with respect to the carbon four-membered ring (in a case where the benzene ring having R⁵ and the benzene ring having R⁶ in Formula (1) have the same chemical structure), the compound represented by Formula (A) can be reacted with the compound represented by Formula (B), but it is preferable to react two molecules of the compound represented by Formula (A) with the squaric acid (the compound represented by Formula (A) with the compound represented by Formula (B1) described later). On the other hand, in a case where the squarylium compound represented by Formula (1) has an asymmetric chemical structure with respect to the carbon four-membered ring (in a case where the benzene ring having R⁵ and the benzene ring having R⁶ in Formula (1) have different chemical structures), it is preferable to react the compound represented by Formula (A) with the compound represented by Formula (B).

Conditions for reacting the compound represented by Formula (A) with the squaric acid (dehydration-condensation reaction) are not particularly limited as long as the reaction proceeds, and can be appropriately set.

An amount of the compound represented by Formula (A) to be used is 2 mol with respect to 1 mol of the squaric acid in terms of stoichiometry, but it is preferably 1.5 to 2.5 mol in practice.

A reaction temperature is preferably equal to or higher than a boiling point (reflux temperature) of a solvent described later, and for example, it is preferably 50° C. to 150° C. and more preferably 80° C. to 120° C. A reaction time can be, for example, 0.5 to 20 hours.

The reaction is usually carried out in a solvent. The solvent to be used is not particularly limited as long as it is a solvent which does not inhibit the reaction. Among these, a solvent that co-boils with water which is by-produced with the progress of the reaction is preferable, and preferred examples thereof include an alcohol solvent having 1 to 6 carbon atoms, aromatic hydrocarbon solvent such as benzene, toluene, and xylene, and a mixed solvent thereof.

In the reaction, it is preferable to exclude and separate the by-produced water from the reaction system, and a normal apparatus, for example, a Dean Stark apparatus can be used in a case of heating and refluxing.

After the reaction, in a case where the produced squarylium compound is dissolved in a reaction solution, the squarylium compound can be obtained as a precipitate by diluting the reaction solution with an alcohol solvent or the like or by cooling the reaction solution. The precipitate can also be purified by a conventional purification method.

For the reaction conditions, post-treatment, and the like, known synthesis methods can be appropriately referred to.

Conditions for reacting the compound represented by Formula (A) with the compound represented by Formula (B) are not particularly limited, and can be appropriately set. Examples thereof include the conditions for reacting the compound represented by Formula (A) with the squaric acid. For the reaction conditions, post-treatment, and the like, known synthesis methods can be appropriately referred to.

The compound represented by Formula (B) can be synthesized by reacting a compound represented by Formula (B1) with a compound represented by Formula (B2).

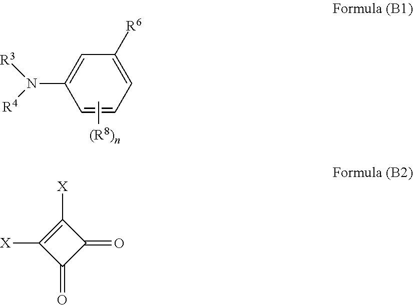

In Formula (B1), each reference numeral is the same as the corresponding reference numeral in Formula (1) described above.

In Formula (B2), X represents an alkoxy group or a halogen atom. The alkoxy group which can be adopted as X is not particularly limited, and examples thereof include the alkoxy group as the substituent X that may be included in the alkyl group or the like, which can be adopted as R¹, and among these, an alkoxy group having 1 to 8 carbon atoms is preferable and an alkoxy group having 1 to 4 carbon atoms is more preferable. Examples of the halogen atom which can be adopted as X include the halogen atom in the substituent X, and a chlorine atom is preferable. X is preferably a methoxy group, an ethoxy group, or a chlorine atom, and two X's may be the same or different from each other.

Conditions for reacting the compound represented by Formula (B1) with the compound represented by Formula (B2) are not particularly limited as long as the reaction proceeds, and can be appropriately set.

An amount of the compound represented by Formula (B2) to be used is 1 mol with respect to 1 mol of the compound represented by Formula (B1) in terms of stoichiometry, but it is preferably 0.8 to 1.2 mol in practice.

A reaction temperature is preferably 20° C. to 150° C., and more preferably 50° C. to 120° C. A reaction time can be, for example, 0.5 to 20 hours.

The reaction is usually carried out in a solvent. The solvent to be used is not particularly limited as long as it is a solvent which does not inhibit the reaction, and preferred examples thereof include the above-described aromatic hydrocarbon solvent.

After completion of the reaction between the compound represented by Formula (B1) and the compound represented by Formula (B2), the compound represented by Formula (B) can be obtained by subjecting the obtained compound to a hydrolysis reaction, for example, in water in the presence of an organic acid such as acetic acid or an inorganic acid such as hydrochloric acid with heating as necessary.

The obtained compound can also be purified by a conventional purification method.

For the reaction conditions, post-treatment, and the like, known synthesis methods can be appropriately referred to.

The squarylium compound represented by Formula (1), Formula (2), Formula (4), or Formula (5) described above can be synthesized by the above-described preferred producing methods. In a case where the squarylium compound represented by Formula (4) or Formula (5) is synthesized, each compound represented by Formula (A), Formula (B), or Formula (B1) described above may not have the branched alkyl group having 4 or more carbon atoms.

In the above-described preferred producing methods, the above-described squarylium compound represented by Formula (3) can be synthesized by introducing the above-described group represented by Formula (4M) into each compound represented by Formula (A), Formula (B), or Formula (B1) described above by a conventional method.

<Resin>

The resin composition according to the embodiment of the present invention contains a resin (binder) (binder may include any conventional component in addition to a polymer; hereinafter, may be referred to as a “binder resin”).

The resin used in the present invention is preferably transparent. Here, the transparent resin refers to a resin having total light transmittance, measured by forming a 1 mm-thick test piece, of usually 70% or more, preferably 80% or more, and more preferably 90% or more.

The resin used as the binder of the resin composition according to the embodiment of the present invention is not particularly limited, and an ordinary resin used as a component of the optical filter can be applied without particular limitation. In addition, the resin can be appropriately selected from resins satisfying various physical properties such as transparency, refractive index, and workability required according to the application or purpose. The resin may be a thermoplastic resin or a thermosetting resin. Examples of the resin include a poly(meth)acrylic resin, an epoxy resin, an ene-thiol resin, a polycarbonate resin, a polyether resin, a polyarylate resin, a polysulfone resin, a polyethersulfone resin, a polyphenylene resin, a polyarylene ether phosphine oxide resin, a polyimide resin, a polyamideimide resin, a polyolefin resin, a cycloolefin resin (cyclic olefin resin), a polyester resin, a polystyrene resin, a polyurethane resin, a polythiourethane resin, a cellulose acylate resin, and an episulfide resin. Since the squarylium compound according to the present invention exhibits a certain degree of compatibility with a hydrophobic resin, a resin exhibiting hydrophobicity can also be used as the resin to be used in combination.

Among the above, preferred examples of the resin contained in the resin composition include a polystyrene resin, a cellulose acylate resin, a poly(meth)acrylic resin, a polyester resin, a cycloolefin resin, and a polycarbonate resin, and from the viewpoint of further reducing fluorescence quantum yield, a polystyrene resin or a cycloolefin resin is preferable.

Both the above-described squarylium compound represented by Formula (1) and the above-described squarylium compound represented by Formula (3) can be used in appropriate combination with each of the above-described resins.

As an example of the combination of the squarylium compound and the resin, for example, from the viewpoint of compatibility with the resin, the above-described squarylium compound represented by Formula (1) is preferably combined with, among the above-described resins, a poly(meth)acrylic resin, a polystyrene resin, a cellulose acylate resin, a cycloolefin resin, a polycarbonate resin, a polyester resin, or the like. In addition, for example, from the viewpoint of exhibiting high light resistance, the above-described squarylium compound represented by Formula (3) is preferably combined with, among the above-described resins, a hydrophobic resin, and specifically, it is preferable to be combined with a polystyrene resin, a cycloolefin resin, or the like.

(Polystyrene Resin)

A polystyrene included in the polystyrene resin refers to a copolymer including 50% by mass or more of a styrene component. In the present invention, only one polystyrene may be used or two or more polystyrenes may be used in combination. Here, the styrene component refers to a constitutional unit derived from a monomer having a styrene skeleton in the structure.

For the purpose of controlling a photoelastic coefficient of a resin composition or an optical filter to be preferable and controlling hygroscopicity of the resin composition or the optical filter to be preferable, the polystyrene more preferably includes 70% by mass or more of the styrene component and still more preferably includes 85% by mass or more of the styrene component. In addition, it is preferable that the polystyrene is composed of only the styrene component.

Examples of the polystyrene include a homopolymer of a styrene compound and a copolymer of two or more styrene compounds. Here, the styrene compound refers to a compound having a styrene skeleton in the structure and also refers to a compound having, in addition to styrene, a substituent introduced to styrene, preferably a portion other than an ethylenically unsaturated bond of styrene. Examples of the styrene compound include styrene; alkyl styrenes such as α-methyl styrene, o-methyl styrene, m-methyl styrene, p-methyl styrene, 3,5-dimethyl styrene, 2,4-dimethyl styrene, o-ethyl styrene, p-ethyl styrene, and tert-butyl styrene; and substituted styrenes in which a hydroxyl group, an alkoxy group, a carboxy group, a halogen, or the like is introduced to a benzene nucleus of styrene, such as hydroxy styrene, tert-butoxy styrene, vinyl benzoic acid, o-chlorostyrene, and p-chlorostyrene. Among these, from the viewpoint of easy procurement, material costs, and the like, the polystyrene used in the present invention is preferably a homopolymer of styrene (that is, polystyrene).

In addition, constitutional components other than the styrene component, which are included in the above-described polystyrene, are not particularly limited. That is, the polystyrene may be a styrene-diene copolymer or a styrene-polymerizable unsaturated carboxylate ester copolymer. In addition, it is also possible to use a mixture of polystyrene and synthetic rubber (for example, polybutadiene, polyisoprene, and the like). In addition, high impact polystyrene (HIPS) obtained by graft-polymerizing styrene to synthetic rubber is also preferable. In addition, polystyrene (referred to as graft-type high impact polystyrene “graft HIPS”) obtained by dispersing a rubber-form elastic body in a continuous phase of a polymer including the styrene component (for example, a copolymer of the styrene component and a (meth)acrylate ester component) and graft-polymerizing the copolymer to the rubber-form elastic body is also preferable. Furthermore, so-called styrene-based elastomers can also be suitably used.

In addition, the above-described polystyrene may be hydrogenated (may be a hydrogenated polystyrene). The hydrogenated polystyrene is not particularly limited, but is preferably hydrogenated styrene-diene-based copolymers such as a hydrogenated styrene-butadiene-styrene block copolymer (SEBS) and a hydrogenated styrene-isoprene-styrene block copolymer (SEPS), which are resins obtained by adding hydrogen to SBS or SIS. The hydrogenated polystyrene may be used singly or in combination of two or more thereof.

The molecular weight of the polystyrene used in the present invention is appropriately selected depending on the purpose of use, but is in a range of, based on mass average molecular weight (in terms of standard polystyrene) measured by gel permeation chromatography of a tetrahydrofuran solution (in a case where the polymer is not dissolved, toluene solution), usually 5,000 to 500,000, preferably 8,000 to 200,000, and more preferably 10,000 to 100,000. A polymer having a molecular weight within the above-described range is capable of satisfying both the mechanical strength and molding workability of a molded product at a high level in a well-balanced manner.

As the polystyrene, a plurality of types of polystyrene having different compositions, molecular weights, and the like can be used in combination.

The polystyrene resin can be obtained by a known anion, massive, suspension, emulsification, or solution polymerization method. In addition, in the polystyrene resin, an unsaturated double bond of a conjugated diene or of a benzene ring of a styrene monomer may be hydrogenated. The hydrogenation rate can be measured by a nuclear magnetic resonance device (NMR).

As the polystyrene resin, a commercially available product may be used, and examples thereof include “CLEAREN 530L”, “CLEAREN 730L” manufactured by Denka Company Limited, “TUFPRENE 1265”, “ASAPRENE T411” manufactured by Asahi Kasei Corporation, “KRATON D1102A”, “KRATON D1116A” manufactured by Kraton Corporation, “STYROLUX S”, “STYROLUX T” manufactured by INEOS Styrolution Group GmbH, “ASAFLEX 840”, “ASAFLEX 860” manufactured by Asahi Kasei Corporation (all of which is SBS), “679”, “HF77”, “SGP-10” manufactured by PS Japan Corporation, “DICSTYRENE XC-515”, “DICSTYRENE XC-535” manufactured by DIC Corporation (all of which is general-purpose polystyrene; GPPS), “475D”, “H0103”, “HT478” manufactured by PS Japan Corporation, and “DICSTYRENE GH-8300-5” manufactured by DIC Corporation (all of which is HIPS). Examples of the hydrogenated polystyrene resin include “TUFTEC H Series” manufactured by Asahi Kasei Corporation, “KRATON G Series” manufactured by Shell Japan Limited (all of which is SEBS), “DYNARON” manufactured by JSR Corporation (hydrogenated styrene-butadiene random copolymer), and “SEPTON” manufactured by Kuraray Co., Ltd. (SEPS). In addition, examples of a modified polystyrene resin include “TUFTEC M Series” manufactured by Asahi Kasei Corporation, “EPOFRIEND” manufactured by Daicel Corporation, “polar group-modified DYNARON” manufactured by JSR Corporation, and “RESEDA” manufactured by Toagosei Co., Ltd.

(Cycloolefin Resin)

A cyclic olefin compound forming a cycloolefin polymer (also referred to as a cyclic polyolefin) included in the cycloolefin resin is not particularly limited as long as the cyclic olefin compound is a compound having a ring structure including a carbon-carbon double bond, and examples thereof include a norbornene compound, a monocyclic olefin compound other than the norbornene compound, a cyclic conjugated diene compound, and a vinyl alicyclic hydrocarbon compound.

Examples of the cycloolefin polymer included in the cycloolefin resin include (R1) polymers including a structural unit derived from a norbornene compound, (R2) polymers including a structural unit derived from a monocyclic olefin compound other than the norbornene compound, (R3) polymers including a structural unit derived from a cyclic conjugated diene compound, (R4) polymers including a structural unit derived from a vinyl alicyclic hydrocarbon compound, and hydrides of polymers including a structural unit derived from each of the compounds (R1) to (R4). In the present invention, the polymer including a structural unit derived from a norbornene compound and the polymer including a structural unit derived from a monocyclic olefin compound include ring-opening polymers of the respective compounds.

The cycloolefin polymer is not particularly limited, but is preferably a polymer having a structural unit derived from a norbornene compound, which is represented by General Formula (A-II) or (A-III). The polymer having the structural unit represented by General Formula (A-II) is an addition polymer of a norbornene compound, and the polymer having the structural unit represented by General Formula (A-III) is a ring-opening polymer of a norbornene compound.

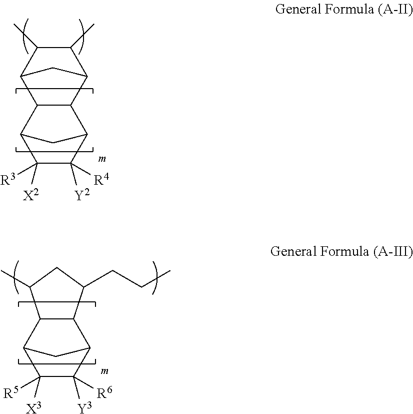

In General Formula (A-II) or (A-III), m represents an integer of 0 to 4 and is preferably 0 or 1.

R³ to R⁶ in Formula (A-II) or (A-III) each independently represent a hydrogen atom or a hydrocarbon group having 1 to 10 carbon atoms.

In the present invention, the hydrocarbon group is not particularly limited as long as the hydrocarbon group is a group consisting of a carbon atom and a hydrogen atom, and examples thereof include an alkyl group, an alkenyl group, an alkynyl group, and an aryl group (aromatic hydrocarbon group). Among these, an alkyl group or an aryl group is preferable.

X² and X³, Y² and Y³ each independently represent a hydrogen atom, a hydrocarbon group having 1 to 10 carbon atoms, a halogen atom, a hydrocarbon group having 1 to 10 carbon atoms, which is substituted with a halogen atom, —(CH₂)nCOOR¹¹, —(CH₂)nOCOR¹², —(CH₂)nNCO, —(CH₂)nNO₂, —(CH₂)nCN, —(CH₂)nCONR¹³R¹⁴, —(CH₂)nNR¹³R¹⁴, —(CH₂)nOZ, —(CH₂)nW, or (—CO)₂O or (—CO)₂NR¹⁵ which is formed by bonding X² and Y², or X³ and Y³.

Here, R¹¹ to R¹⁵ in the above-described groups which can be adopted as X², X³, Y², and Y³ each independently represent a hydrogen atom or a hydrocarbon group having 1 to 20 carbon atoms, Z represents a hydrocarbon group or a hydrocarbon group substituted with halogen, and W represents Si(R¹⁶)ₚD₍₃₋ₚ₎ (R¹⁶ represents a hydrocarbon group having 1 to 10 carbon atoms, and D represents a halogen atom, —OCOR¹⁷, or —OR¹⁷ (R¹⁷ represents a hydrocarbon group having 1 to 10 carbon atoms), and p represents an integer of 0 to 3). n represents an integer of 0 to 10, and is preferably 0 to 8 and more preferably 0 to 6.

In General Formula (A-II) or (A-III), R³ to R⁶ are each preferably a hydrogen atom or —CH₃, and from the viewpoint of moisture permeability, still more preferably a hydrogen atom.

Each of X² and X³ is preferably a hydrogen atom, —CH₃, or —C₂H₅, and from the viewpoint of moisture permeability, still more preferably a hydrogen atom.

Each of Y² and Y³ is preferably a hydrogen atom, a halogen atom (particularly a chlorine atom), or —(CH₂)nCOOR¹¹ (particularly —COOCH₃), and from the viewpoint of moisture permeability, still more preferably a hydrogen atom.

Other groups are appropriately selected.

The polymer having the structural unit represented by General Formula (A-II) or (A-III) may further include at least one kind of a structural unit represented by General Formula (A-I).

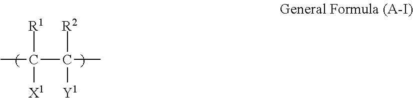

R¹ and R² in General Formula (A-I) each independently represent a hydrogen atom or a hydrocarbon group having 1 to 10 carbon atoms, and X¹ and Y¹ each independently represent a hydrogen atom, a hydrocarbon group having 1 to 10 carbon atoms, a halogen atom, a hydrocarbon group having 1 to 10 carbon atoms, which is substituted with a halogen atom, —(CH₂)nCOOR¹¹, —(CH₂)nOCOR¹², —(CH₂)nNCO, —(CH₂)nNO₂, —(CH₂)nCN, —(CH₂)nCONR¹³R¹⁴, —(CH₂)nNR¹³R¹⁴, —(CH₂)nOZ, —(CH₂)nW, or (—CO)₂O or (—CO)₂NR¹⁵ which is formed by bonding X¹ and Y¹.

Here, R¹¹ to R¹⁵ in the above-described groups which can be adopted as X¹ and Y¹ each independently represent a hydrogen atom or a hydrocarbon group having 1 to 20 carbon atoms, Z represents a hydrocarbon group or a hydrocarbon group substituted with halogen, and W represents Si(R¹⁶)ₚD₍₃₋ₚ₎ (R¹⁶ represents a hydrocarbon group having 1 to 10 carbon atoms, and D represents a halogen atom, —OCOR¹⁷, or —OR¹⁷ (R¹⁷ represents a hydrocarbon group having 1 to 10 carbon atoms), and p represents an integer of 0 to 3). n represents an integer of 0 to 10.

From the viewpoint of adhesiveness to a polarizer, the content of the above-described structural unit derived from a norbornene compound in the cyclic polyolefin having the structural unit represented by General Formula (A-II) or (A-III) is preferably 90% by mass or less, more preferably 30% to 85% by mass, still more preferably 50% to 79% by mass, and most preferably 60% to 75% by mass with respect to the total mass of the cyclic polyolefin. Here, the proportion of the structural unit derived from a norbornene compound represents the average value in the cyclic polyolefin.

Addition (co)polymers of a norbornene compound are described in JP1998-7732A (JP-H10-7732A), JP2002-504184A, US2004/229157A1, WO2004/070463A, or the like, and the contents thereof can be referred to as appropriate and are incorporated as they are as part of the description of the present specification.

The polymer of a norbornene compound is obtained by an addition polymerization of norbornene compounds (for example, polycyclic unsaturated compounds of norbornene).

In addition, examples of the polymer of a norbornene compound include copolymers obtained by an addition copolymerization of, as necessary, a norbornene compound, and olefin such as ethylene, propylene, and butene, conjugated diene such as butadiene and isoprene, unconjugated diene such as ethylidene norbornene, or an ethylenically unsaturated compound such as acrylonitrile, acrylic acid, methacrylic acid, maleic acid anhydride, acrylic acid ester, methacrylic acid ester, maleimide, vinyl acetate, and vinyl chloride. Among these, a copolymer with ethylene is preferable.

Examples of the above-described addition (co)polymers of a norbornene compound include APL8008T (Tg: 70° C.), APL6011T (Tg: 105° C.), APL6013T (Tg: 125° C.), and APL6015T (Tg: 145° C.) which are sold by Mitsui Chemicals, Inc. under a trade name of APL and have different glass transition temperatures (Tg). In addition, pellets such as TOPAS8007, TOPAS6013, and TOPAS6015 are commercially available from Polyplastics Co., Ltd. Furthermore, Appear3000 is commercially available from Film Ferrania S. R. L.

As the above-described polymer of a norbornene compound, a commercially available product can be used. For example, polymers are commercially available from JSR Corporation under a trade name of Arton, specifically Arton G, F, or RX4500, and polymers are commercially available from Zeon Corporation under a trade name of Zeonor ZF14, ZF16, Zeonex 250, or Zeonex 280.

The hydride of the polymer of a norbornene compound can be synthesized by an addition polymerization or a ring-opening metathesis polymerization of a norbornene compound or the like and then an addition of hydrogen. Synthesis methods are described in, for example, JP1989-240517A (JP-H1-240517A), JP1995-196736A (JP-H7-196736A), JP1985-26024A (JP-S60-26024A), JP1987-19801A (JP-S62-19801A), JP2003-159767A, JP2004-309979A, and the like.

The molecular weight of the cycloolefin polymer used in the present invention is appropriately selected depending on the purpose of use, but is in a range of, based on mass average molecular weight in terms of polyisoprene or polystyrene measured by gel permeation chromatography of a cyclohexane solution (in a case where the polymer is not dissolved, toluene solution), usually 5,000 to 500,000, preferably 8,000 to 200,000, and more preferably 10,000 to 100,000. A polymer having a molecular weight within the above-described range is capable of satisfying both the mechanical strength and molding workability of a molded product at a high level in a well-balanced manner.

(Poly(Meth)Acrylic Resin)

Examples of a poly(meth)acrylic polymer included in the poly(meth)acrylic resin include a polymer having a constitutional unit derived from (meth)acrylic acid and/or an ester thereof. Specific examples thereof include polymers obtained by polymerizing at least one compound selected from the group consisting of (meth)acrylic acid, (meth)acrylic acid ester, (meth)acrylamide, and (meth)acrylonitrile.

Preferred examples of the poly(meth)acrylic polymer include a homopolymer and copolymer obtained by (co)polymerizing a compound represented by General Formula A1 as a monomer component.

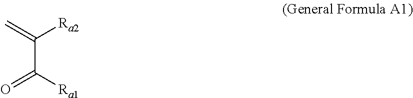

In General Formula A1, Rₐ₁ represents a hydroxy group, a substituted or unsubstituted alkoxy group, a substituted or unsubstituted aryloxy group, a substituted or unsubstituted amino group, a substituted or unsubstituted alkyl group, a substituted or unsubstituted aryl group, or a substituted or unsubstituted heterocyclic group. Rₐ₁ is preferably a hydroxy group, a substituted or unsubstituted alkoxy group, or a substituted or unsubstituted aryloxy group, and more preferably a hydroxy group, a substituted or unsubstituted alkoxy group having 1 to 18 carbon atoms, or a substituted or unsubstituted aryloxy group having 6 to 24 carbon atoms.

Rₐ₂ represents a hydrogen atom, a methyl group, or an alkyl group having 2 or more carbon atoms. Rₐ₂ is preferably a hydrogen atom or a methyl group.

Examples of a preferred combination of Rₐ₁ and Rₐ₂ in General Formula A1 include a combination in which Rₐ₁ is a hydroxy group, a substituted or unsubstituted alkoxy group having 1 to 18 carbon atoms, or a substituted or unsubstituted aryloxy group having 6 to 24 carbon atoms, and Rₐ₂ is a hydrogen atom or a methyl group.

Specific examples of the compound represented by General Formula A1 include the following.
Acrylic acid compound or methacrylic acid compound
Acrylic acid ester compound

methyl acrylate, ethyl acrylate, (n- or i-)propyl acrylate, (n-, i-, sec- or t-)butyl acrylate, amyl acrylate, 2-ethylhexyl acrylate, dodecyl acrylate, chloroethyl acrylate, 2-hydroxyethyl acrylate, 2-hydroxypropyl acrylate, 2-hydroxypentyl acrylate, cyclohexyl acrylate, allyl acrylate, trimethylolpropane monoacrylate, pentaerythritol monoacrylate, benzyl acrylate, methoxybenzyl acrylate, chlorobenzyl acrylate, hydroxybenzyl acrylate, hydroxyphenethyl acrylate, dihydroxyphenethyl acrylate, furfuryl acrylate, tetrahydrofurfuryl acrylate, phenyl acrylate, hydroxyphenyl acrylate, chlorophenyl acrylate, sulfamoylphenyl acrylate, and 2-(hydroxyphenylcarbonyloxy)ethyl acrylate
Methacrylic acid ester compound

methyl methacrylate, ethyl methacrylate, (n- or i-)propyl methacrylate, (n-, i-, sec- or t-)butyl methacrylate, amyl methacrylate, 2-ethylhexyl methacrylate, dodecyl methacrylate, chloroethyl methacrylate, 2-hydroxyethyl methacrylate, 2-hydroxypropyl methacrylate, 2-hydroxypentyl methacrylate, cyclohexyl methacrylate, allyl methacrylate, trimethylolpropane monomethacrylate, pentaerythritol monomethacrylate, benzyl methacrylate, methoxybenzyl methacrylate, chlorobenzyl methacrylate, hydroxybenzyl methacrylate, hydroxyphenethyl methacrylate, dihydroxyphenethyl methacrylate, furfuryl methacrylate, tetrahydrofurfuryl methacrylate, phenyl methacrylate, hydroxyphenyl methacrylate, chlorophenyl methacrylate, sulfamoylphenyl methacrylate, and 2-(hydroxyphenylcarbonyloxy)ethyl methacrylate
Acrylamide compound

acrylamide, N-methylacrylamide, N-ethylacrylamide, N-propylacrylamide, N-butylacrylamide, N-benzylacrylamide, N-hydroxyethylacrylamide, N-phenylacrylamide, N-tolylacrylamide, N-(hydroxyphenyl)acrylamide, N-(sulfamoylphenyl)acrylamide, N-(phenylsulfonyl)acrylamide, N-(tolylsulfonyl)acrylamide, N,N-dimethylacrylamide, N-methyl-N-phenylacrylamide, and N-hydroxyethyl-N-methylacrylamide
Methacrylamide compound

methacrylamide, N-methylmethacrylamide, N-ethylmethacrylamide, N-propylmethacrylamide, N-butylmethacrylamide, N-benzylmethacrylamide, N-hydroxyethylmethacrylamide, N-phenylmethacrylamide, N-tolylmethacrylamide, N-(hydroxyphenyl)methacrylamide, N-(sulfamoylphenyl)methacrylamide, N-(phenylsulfonyl)methacrylamide, N-(tolylsulfonyl)methacrylamide, N,N-dimethylmethacrylamide, N-methyl-N-phenylmethacrylamide, and N-hydroxyethyl-N-methylmethacrylamide

The poly(meth)acrylic polymer is preferably a homopolymer obtained by polymerizing the above-described compound represented by General Formula A1 or a 2- to 4-component, preferably 2- or 3-component copolymer, which is obtained by copolymerizing the compound represented by General Formula A1 at a molar ratio of 10% to 90%, preferably 20% to 80% with another compound or a further compound represented by General Formula A1. Examples of the another compound described above include a substituted or unsubstituted styrene compound and acrylonitrile.

The poly(meth)acrylic polymer is preferably a homopolymer obtained by polymerizing acrylic acid ester or methacrylic acid ester having 4 to 24 carbon atoms, a copolymer obtained by polymerizing two or more compounds represented by General Formula A1 described above, or 2- or 3-component copolymer having acrylic acid ester or methacrylic acid ester at a molar ratio of 10% to 90%.

The molecular weight of the poly(meth)acrylic polymer is appropriately selected depending on the purpose of use, but is in a range of, based on mass average molecular weight in terms of polyisoprene or polystyrene measured by gel permeation chromatography of a cyclohexane solution (in a case where the polymer is not dissolved, toluene solution), usually 5,000 to 500,000, preferably 8,000 to 200,000, and more preferably 10,000 to 100,000. A polymer having a molecular weight within the above-described range is capable of satisfying both the mechanical strength and molding workability of a molded product at a high level in a well-balanced manner.

(Polyester Resin)

Examples of a polyester polymer included in the polyester resin include polymers obtained by reacting a polyol (such as ethylene glycol, propylene glycol, glycerin, and trimethylolpropane) with a polybasic acid (such as aromatic dicarboxylic acid (for example, terephthalic acid, isophthalic acid, naphthalenedicarboxylic acid, and dicarboxylic acid in which a hydrogen atom of these aromatic rings is replaced with a methyl group, an ethyl group, or a phenyl group), aliphatic dicarboxylic acid having 2 to 20 carbon atoms (for example, adipic acid, sebacic acid, and dodecanedicarboxylic acid), and alicyclic dicarboxylic acid (for example, cyclohexanedicarboxylic acid)); and polymers obtained by ring-opening polymerization of a cyclic ester compound such as caprolactone monomers (for example, polycaprolactone). In addition, as the polyester polymer, the content of “polyester” described in JP2009-096971A can be appropriately referred to, and the contents thereof are incorporated as they are as part of the description of the present specification.

(Cellulose Acylate Resin)

A cellulose acylate included in the cellulose acylate resin is not particularly limited, and a commonly used cellulose acylate can be appropriately used. For example, cellulose acylates described in paragraphs 0016 to 0021 of JP2012-215689A are preferably used, and the contents described in the paragraphs are incorporated as they are as part of the description of the present specification.

(Polycarbonate Resin)

A polycarbonate included in the polycarbonate resin includes the following polyhydric phenol compound and a carbonic ester compound such as bisalkyl carbonate, bisaryl carbonate, and phosgene.

Examples of the polyhydric phenol compound include hydroquinone, resorcinol, 4,4′-dihydroxydiphenyl, bis(4-hydroxyphenyl)methane, 1,1-bis(4-hydroxyphenyl)ethane, 1,1-bis(4-hydroxyphenyl)-1-phenylethane, bisphenol A, bisphenol C, bisphenol E, bisphenol F, bisphenol M, bisphenol P, bisphenol S, bisphenol Z, 2,2-bis(3-methyl-4-hydroxyphenyl)propane, 1,1-bis(4-hydroxyphenyl)cyclohexane, 2,2-bis(3-phenyl-4-hydroxyphenyl)propane, 2,2-bis(3-isopropyl-4-hydroxyphenyl)propane, 2,2-bis(4-hydroxyphenyl)butane, 2,2-bis(3,5-dimethyl-4-hydroxyphenyl)propane, 2,2-bis(3,5-dibromo-4-hydroxyphenyl)propane, 4,4′-dihydroxydiphenyl sulfone, 4,4′-dihydroxydiphenyl sulfoxide, 4,4′-dihydroxydiphenyl sulfide, 3,3′-dimethyl-4,4′-dihydroxydiphenyl sulfide, and 4,4′-dihydroxydiphenyl oxide. Among the above, the polyhydric phenol compound is preferably hydroquinone, resorcinol, 4,4′-dihydroxydiphenyl, or bisphenol A.

Examples of the carbonic ester compound include phosgene, diphenyl carbonate, bis(chlorophenyl) carbonate, dinaphthyl carbonate, bis(diphenyl) carbonate, dimethyl carbonate, diethyl carbonate, and dibutyl carbonate.

Among the above, the carbonic ester compound is preferably phosgene, bis(diphenyl) carbonate, dimethyl carbonate, or diethyl carbonate.

In the polycarbonate, preferred examples of a combination of monomers and the polymer include bisphenol A polycarbonate using bisphenol A as the polyhydric phenol compound and phosgene as the carbonic ester compound.

As the polycarbonate, a commercially available product may be used, and examples thereof include Panlite (registered trademark) L-1250WP (trade name; aromatic polycarbonate resin powder, manufactured by TEIJIN LIMITED.), Panlite (registered trademark) SP-1516 (trade name, manufactured by TEIJIN LIMITED.), Iupizeta (registered trademark) EP-5000 (trade name, manufactured by MITSUBISHI GAS CHEMICAL COMPANY, INC.), Iupizeta (registered trademark) EP-4000 (trade name, manufactured by MITSUBISHI GAS CHEMICAL COMPANY, INC.), and Calibre 301-30 (SD Calibre 301-30) (trade name, manufactured by Sumika Polycarbonate Ltd.).

(Polythiourethane Resin)

The polythiourethane resin may be a polymer having a thiourethane bond in which at least one oxygen atom in a urethane bond (—NR^{T}—CO—O—) is substituted with a sulfur atom, and examples thereof include a polymer having —NR^{T}—CS—O—, —NR^{T}—CO—S—, or —NR^{T}—CS—S—. Here, R^{T} represents a hydrogen atom or a substituent.

The resin used in the resin composition according to the embodiment of the present invention has a glass transition temperature (Tg) of preferably −80° C. to 200° C. and more preferably −30° C. to 180° C. In a case where the resin composition contains a resin exhibiting Tg in the above-described range, it is possible to produce an optical filter having moderate softness and hardness. The glass transition temperature of the resin can be appropriately adjusted depending on the composition of the resin (type or content of the constitutional components) and the like. The glass transition temperature of the resin can be measured by a method described in Guide to Instrumental Analysis (publisher: Kagaku-Dojin Publishing Company, INC) using a differential scanning calorimeter (DSC).

From the viewpoint of sharpness of absorption waveform and light resistance, the resin composition according to the embodiment of the present invention contains the binder resin in an amount of preferably 50% by mass or more, more preferably 70% by mass or more, and particularly preferably 90% by mass or more with respect to the total solid content (specifically, with respect to the components excluding an organic solvent described later).

The resin composition may contain two or more kinds of binder resins, and binder resins having different compositional ratios and/or molecular weights may be used in combination. In this case, the total content of the respective binder resins is within the above-described range.

<Additive>

The resin composition according to the embodiment of the present invention may contain an additive as long as the effects of the present invention are not impaired. For example, the resin composition according to the embodiment of the present invention may include an additive which can be generally blended in a plastic film as necessary. Examples of the additive include an antioxidant, a heat stabilizer, a light stabilizer, an ultraviolet absorber, an antistatic agent, a lubricant, a plasticizer, and a filler, and the content thereof can be selected within a range which does not impair the object of the present invention. In addition, examples of the additive include a known plasticizer, an organic acid, a polymer, a retardation adjuster, an ultraviolet absorber, an antioxidant, and a matting agent. With regard to these compounds, reference can be made to the description in paragraphs “0062” to “0097” of JP2012-155287A, and the contents of which are incorporated herein by reference. In addition, examples of the additive include a peeling accelerator, an organic acid, and a polyvalent carboxylic acid derivative. With regard to these compounds, reference can be made to the description in paragraphs “0212” to “0219” of WO2015/005398A, and the contents of which are incorporated herein by reference. Furthermore, examples of the additive include a radical scavenger and a deterioration inhibitor which will be described later.

The content of the additive (in a case where the resin composition contains two or more kinds of additives, total content thereof) is preferably 50 parts by mass or less, more preferably 30 parts by mass or less, and still more preferably 5 to 30 parts by mass with respect to 100 parts by mass of the binder resin.

(Antioxidant)

A preferred example of the additive includes an antioxidant. With regard to the antioxidant, reference can be made to the description in paragraphs “0143” to “0165” of WO2015/005398A, and the contents of which are incorporated herein by reference.

(Radical Scavenger)

A preferred example of the additive includes a radical scavenger. With regard to the radical scavenger, reference can be made to the description in paragraphs “0166” to “0199” of WO2015/005398A, and the contents of which are incorporated herein by reference.

(Deterioration Inhibitor)

A preferred example of the additive includes a deterioration inhibitor. With regard to the deterioration inhibitor, reference can be made to the description in paragraphs “0205” and “0206” of WO2015/005398A, and the contents of which are incorporated herein by reference.

(Ultraviolet Absorber)

In the present invention, from the viewpoint of preventing deterioration, an ultraviolet absorber may be added to the optical filter. From the viewpoint of excellent absorption capacity of ultraviolet rays with a wavelength of 370 nm or less and good liquid crystal display properties, an ultraviolet absorber having a small absorption of visible light with a wavelength of 400 nm or more is preferably used. Specific examples of the ultraviolet absorber preferably used in the present invention include a hindered phenol-based compound, a hydroxybenzophenone-based compound, a benzotriazole-based compound, a salicylic acid ester-based compound, a benzophenone-based compound, a cyanoacrylate-based compound, and a nickel complex salt compound.

Examples of the hindered phenol-based compound include 2,6-di-tert-butyl-p-cresol, pentaerythrityl-tetrakis[3-(3,5-di-tert-butyl-4-hydroxyphenyl)propionate], N,N′-hexamethylenebis(3,5-di-tert-butyl-4-hydroxy-hydrocinnamide), 1,3,5-trimethyl-2,4,6-tris(3,5-di-tert-butyl-4-hydroxybenzyl)benzene, and tris-(3,5-di-tert-butyl-4-hydroxybenzyl)-isocyanurate. Examples of the benzotriazole-based compound include 2-(2′-hydroxy-5′-methylphenyl)benzotriazole, 2,2-methylenebis(4-(1,1,3,3-tetramethylbutyl)-6-(2H-benzotriazol-2-yl)phenol), (2,4-bis-(n-octylthio)-6-(4-hydroxy-3,5-di-tert-butylanilino)-1,3,5-triazine, triethylene glycol-bis[3-(3-tert-butyl-5-methyl-4-hydroxyphenyl)propionate], N,N′-hexamethylenebis(3,5-di-tert-butyl-4-hydroxy-hydrocinnamide), 1,3,5-trimethyl-2,4,6-tris(3,5-di-tert-butyl-4-hydroxybenzyl)benzene, 2(2′-hydroxy-3′,5′-di-tert-butylphenyl)-5-chlorobenzotriazole, (2(2′-hydroxy-3′,5′-di-tert-amylphenyl)-5-chlorobenzotriazole, 2,6-di-tert-butyl-p-cresol, pentaerythrityl-tetrakis[3-(3,5-di-tert-butyl-4-hydroxyphenyl)propionate], and 2-(2H-benzotriazol-2-yl)-6-(1-methyl-1-phenylethyl)-4-(1,1,3,3-tetramethylbutyl)phenol.

The resin composition according to the embodiment of the present invention can contain various additives, but in a case of being used as a forming material for an optical filter, the resin composition according to the embodiment of the present invention may not contain an antifading agent. In the present invention, the term “not contain an antifading agent” includes a case where a content of the antifading agent is less than a content required to prevent fading of the optical filter (dye contained in the optical filter), for example, a content of less than 1% by mass, preferably less than 0.5% by mass in 100% by mass of the total solid content. The antifading agent is not particularly limited, and examples thereof include a commonly used antifading agent such as an antioxidant described in paragraphs “0143” to “0165” of WO2015/005398A1, a radical scavenger described in paragraphs “0166” to “0199” of WO2015/005398A1, and a deterioration inhibitor described in paragraphs “0205” and “0206” of WO2015/005398A1.

In addition, in a case where the resin composition according to the embodiment of the present invention is used as the forming material for an optical filter, the resin composition according to the embodiment of the present invention may not contain the copper compound disclosed in JP2009-036811A.

<Solvent>

The resin composition according to the embodiment of the present invention can also contain a solvent. In particular, in order to form the coated and dried product described later, it is preferable that the resin composition according to the embodiment of the present invention contains a solvent having a boiling point of 200° C. or lower and the above-described squarylium compound and resin are dissolved in the solvent. Here, the fact that the squarylium compound and resin are dissolved includes an aspect in which all of the squarylium compound and the resin are dissolved in the solvent and an aspect in which a part thereof is not dissolved in the solvent, for example, an aspect in which 0.5% by mass or less of the squarylium compound and the resin is not dissolved in a total of 100% by mass of the squarylium compound and the resin and exists in a solid state.

The boiling point of the solvent can be appropriately determined depending on coating and drying conditions described later, but from the viewpoint of avoiding excessive heating during the drying and saving energy, it is preferably 180° C. or lower and more preferably 160° C. or lower. On the other hand, the lower limit value thereof is not particularly limited, and may be, for example, 60° C. or higher. In the present invention, the boiling point of the solvent is a standard boiling point or a normal boiling point, and means a boiling point under a pressure (normal pressure) of 101,325 Pa.

The organic solvent and the content thereof are the same as those in the following “Method of manufacturing optical filter”.

<Preparation of Resin Composition>

The resin composition according to the embodiment of the present invention can be prepared by a conventional method.

In a case where the resin composition according to the embodiment of the present invention is a simple mixture of the squarylium compound and the resin, the resin composition according to the embodiment of the present invention can be prepared by dry-mixing the squarylium compound and the resin by a conventional method.

In a case where the resin composition according to the embodiment of the present invention is a liquid composition, the resin composition according to the embodiment of the present invention can be prepared by wet-mixing the squarylium compound, the resin, and the solvent by a conventional method.

In a case where the resin composition according to the embodiment of the present invention is a coated and dried product, the resin composition according to the embodiment of the present invention can be prepared by applying and drying the above-described liquid composition on a substrate. The substrate is not particularly limited, and examples thereof include a resin substrate, a glass substrate, a metal substrate, a vapor deposition film, and a surface of a member on which an optical filter is disposed, which will be described later. The method for applying the liquid composition is not particularly limited, and examples thereof include a spray method, a dipping method, a roller coating method, a flow coating method (for example, a solution-casting film forming method described later), a curtain coating method, a bar coating method, a blade coating method, and a spin coating method. Conditions for applying are not particularly limited, and are appropriately set in consideration of an amount of the liquid composition to be applied, a viscosity of the liquid composition, a shape and dimensions of the coated and dried product, and the like. Drying method and conditions are not particularly limited as long as the solvent in the liquid composition can be removed up to the above-described residual amount or less, and are appropriately set. Examples of the heating method include heat drying and blast drying, and heat drying is preferable. A heating temperature in this case is not particularly limited and can be set to a temperature equal to or higher than the boiling point of the solvent at an ambient pressure during the drying, and for example, it can be set to 50° C. to 200° C. under normal pressure.

In a case where the resin composition according to the embodiment of the present invention is a molten mixture, the resin composition according to the embodiment of the present invention can be prepared by mixing the squarylium compound and the resin (including the simple mixture) while heating to melt the resin, and then cooling and solidifying the mixture. A melt-mixing temperature in this case is not particularly limited as long as it is equal to or higher than the melting temperature of the resin, and can be appropriately determined on the type of the resin, the melting point, the glass transition temperature, and the like. For example, the melt-mixing temperature can be 180° C. or higher, preferably 200° C. or higher. The upper limit thereof can be, for example, 400° C. or lower, preferably 350° C. or lower. Melt-mixing method and conditions are appropriately determined, and are usually carried out using various mixers.

In a case of preparing the coated and dried product or the molten mixture, preparation conditions, for example, an applying amount and a cooling method, can be determined so as to have a shape and dimensions according to the application or the like.

The prepared resin composition can also be adjusted to have a shape and dimensions according to the application or the like by a conventional method, for example, a molding method, a dimensional adjustment method, and the like. As the molten mixture, a heat-melting molding method described later, in which melt-solidification and molding are performed, can also be applied.

[Optical Filter]

The resin composition according to the embodiment of the present invention is suitable as a material for forming an optical filter by appropriately performing molding or the like. The optical filter is usually molded into a flat film form, but in the present invention, the optical filter may be molded into various forms such as curved film, powdery, spherical, crushed particles, bulky continuum, fiber-like, tubular, hollow fiber-like, granular, and porous shape, according to a surface condition of a member on which the optical filter is disposed.

The optical filter according to the embodiment of the present invention is formed of the resin composition, coated and dried product, or melt-kneaded product according to the embodiment of the present invention, and has a predetermined shape. The optical filter according to the embodiment of the present invention is preferably formed of the resin composition, coated and dried product, or melt-kneaded product according to the embodiment of the present invention in a film form, and is more preferably a film-like molded product of the resin composition according to the embodiment of the present invention. A content of each component (solid content excluding the organic solvent) in the optical filter is the same as the content in the resin composition according to the embodiment of the present invention (in the solid content).

The optical filter according to the embodiment of the present invention can be suitably used as a light absorption filter (film) which highly absorbs (blocks a passage of) target light having a specific wavelength, such as light of an unnecessary wavelength in incidence light. In addition, the optical filter according to the embodiment of the present invention exhibits the above-described excellent characteristics, can highly absorb (block a passage of) near infrared rays in the above-described wavelength range, and is also excellent in oblique incidence characteristics. Therefore, in addition to the light absorption filter, the optical filter according to the embodiment of the present invention can also be suitably used as a near-infrared cut filter that corrects visibility of a solid-state imaging element which uses, as a light receiving section, a silicon photodiode sensing infrared rays. In a case where the optical filter according to the embodiment of the present invention is applied as the near-infrared cut filter, the optical filter according to the embodiment of the present invention can be used in a normal aspect (usage method and the like), and for example, the description of JP6605039B can be referred to, and the contents thereof are incorporated as they are as part of the description of the present specification.

<Method of Manufacturing Optical Filter>

Hereinafter, a method of manufacturing an optical filter will be described.

The optical filter is not particularly limited as long as the resin composition, coated and dried product, or melt-kneaded product according to the embodiment of the present invention is used, and the optical filter can be appropriately manufactured by an ordinary manufacturing method. For example, the method described in the preparation of the resin composition above can be applied.

(Solution-Casting Film Forming Method)

In a case where the optical filter according to the embodiment of the present invention has a film form, the optical filter can be manufactured using the above-described coated and dried product or molten mixture, and a solution-casting film forming method is one preferred manufacturing aspect. In the solution-casting film forming method, a film can be manufactured using a solution (dope, “liquid composition” as one aspect of the resin composition according to the embodiment of the present invention) prepared by dissolving at least the squarylium compound and the binder resin in an organic solvent.

The organic solvent is not particularly limited as long as it can dissolve the squarylium compound and the binder resin. For example, a solvent selected from an aliphatic hydrocarbon solvent having 6 to 12 carbon atoms, an aromatic hydrocarbon solvent having 6 to 20 carbon atoms, an alcohol solvent having 1 to 4 carbon atoms, an ether solvent having 3 to 12 carbon atoms, a ketone solvent having 3 to 12 carbon atoms, an ester solvent having 3 to 12 carbon atoms, or a halogenated hydrocarbon solvent having 1 to 6 carbon atoms or a mixed solvent of these solvents can be used. Preferred examples of the mixed solvent include a mixed solvent of an aliphatic hydrocarbon solvent or a ketone solvent and an aromatic hydrocarbon solvent.

The aliphatic hydrocarbon solvent, ether solvent, ketone solvent, and ester solvent may have a cyclic structure. In addition, as the above-described organic solvent, a compound having two or more functional groups (that is, —O—, —CO—, and —COO—) of the above-described ether solvent, ketone solvent, and ester solvent (for example, alkylene glycol monoalkyl ether, alkylene glycol dialkyl ether, alkylene glycol monoalkyl ether acetate, and alkylene glycol dialkyl ether acetate) can also be used. The above-described organic solvent may have another functional group such as an alcoholic hydroxyl group. In a case of an organic solvent having two or more kinds of functional groups, it is preferable that the number of carbon atoms of the organic solvent is within the above-described preferred range with regard to the number of carbon atoms of the solvent having any functional group.

With an organic solvent having a boiling point of 200° C. or lower, it is possible to avoid drying at an excessively high drying temperature after coating. A preferred range of the boiling point is as described above.

A content of the binder resin in the solution is preferably adjusted to 1% to 80% by mass and still more preferably 10% to 75% by mass. The above-described optional additive may be added to the organic solvent (main solvent).

The total content of the total solid content in the solution is the sum of the contents of the squarylium compound, the binder resin, and the additive described above, and for example, it is preferably 1% to 80% by mass, more preferably 5% to 75% by mass, and still more preferably 10% to 65% by mass.

With regard to a drying method in the solution-casting film forming method, reference can be made to the description in U.S. Pat. Nos. 2,336,310A, 2,367,603A, 2,492,078A, 2,492,977A, 2,492,978A, 2,607,704A, 2,739,069A, 2,739,070A, GB640731B, GB736892B, JP1970-4554B (JP-S45-4554B), JP1974-5614B (JP-S49-5614B), JP1985-176834A (JP-S60-176834A), JP1985-203430A (JP-S60-203430A), and JP1987-115035A (JP-S62-115035A). Drying on a band can be performed by blowing air or an inert gas such as nitrogen.

It is preferable that the dope is cast onto the band and the solvent is evaporated to form a film. It is preferable that the concentration of the dope before casting is adjusted such that the solid content is in a range of 10% to 40% by mass. It is preferable that the surface of the band is polished off in a state of mirror surface.

It is also possible to cast two or more layers using the prepared solution (dope) to form a film.

In a case of casting a plurality of dopes, for example, cycloolefin resin solutions to form a film having two or more layers, a film may be produced while casting each of the dopes from a plurality of casting ports provided at intervals in a traveling direction of a support, and laminating the solutions. With regard to the methods, for example, methods described in JP1986-158414A (JP-S61-158414A), JP1989-122419A (JP-H1-122419A), and JP1999-198285A (JP-H11-198285A) can be used. In addition, it is also possible to cast the dope from two casting ports to form a film. With regard to the method, for example, methods described in JP1985-27562A (JP-S60-27562A), JP1986-94724A (JP-S61-94724A), JP1986-947245A (JP-S61-947245A), JP1986-104813A (JP-S61-104813A), JP1986-158413A (JP-S61-158413A), and JP1994-134933A (JP-H6-134933A) can be used. Furthermore, a casting method of a resin film described in JP1981-162617A (JP-S56-162617A), in which the flow of a high-viscosity resin solution is wrapped with a low-viscosity resin solution and the high-viscosity and low-viscosity resin solutions are extruded at the same time.

In addition, a film can be produced by, using two casting ports, peeling off a film molded on a support by a first casting port and performing second casting on a side which is in contact with the support surface. Examples thereof include a method described in JP1969-20235B (JP-S44-20235B).

As the solution to be cast, the same solution may be used or two or more different solutions may be used. It is sufficient that, in order to allow a plurality of layers to have a function, a solution corresponding to the function is extruded from each casting port. Furthermore, as the forming of a solution casting film, an aspect in which other functional layers (for example, an adhesive layer, a dye layer, an antistatic layer, an antihalation layer, an ultraviolet absorbing layer, a polarizing layer, and the like) are cast at the same time can be used.

The compound (coloring agent) represented by General Formula (1) can be added to the above-described solution by, for example, mixing with the binder resin in the organic solvent in a case of preparing the dope.

(Drying Treatment)

Steps from casting of the dope to post-drying may be performed under an atmosphere of air or under an atmosphere of inert gas such as nitrogen. A winding machine used for manufacturing the optical filter according to the embodiment of the present invention may be a commonly used winding machine, and the winding can be performed by a winding method such as a constant tension method, a constant torque method, a taper tension method, and a program tension control method with a constant internal stress. As drying conditions, for example, the drying conditions for producing the coated and dried product can be applied.

(Stretching Treatment)

The above-described optical filter can also be subjected to a stretching treatment. It is possible to impart a desired retardation to the optical filter by the stretching treatment. As a stretching direction of the optical filter, any one of a width direction or a longitudinal direction is preferable.

The stretching method in the width direction is described in, for example, JP1987-115035A (JP-S62-115035A), JP1992-152125A (JP-H4-152125A), JP1992-284211A (JP-H4-284211A), JP1992-298310A (JP-H4-298310A), JP1999-48271A (JP-H11-48271A), and the like.

The stretching of the film (optical filter before the stretching treatment) is performed under heating conditions. The film can be stretched during the treatment of drying, which is particularly effective in a case where the solvent remains. In a case of stretching in the longitudinal direction, for example, the film is stretched by adjusting a speed of a film handling roller so that a film winding speed is faster than a film peeling speed. In a case of stretching in the width direction, the film can be stretched by handling the film while holding a width of the film by a tenter and gradually widening a width of the tenter. It is also possible to stretch the film using a stretching machine (preferably monoaxial stretching using a long stretching machine) after drying the film.

The method for molding the optical filter is not particularly limited, and the optical filter can be formed as described above. Furthermore, any of a heat-melting molding method or a solution casting method can be used. The heat-melting molding method can be classified in more detail into an extrusion molding method, a press molding method, an inflation molding method, an injection molding method, a blow molding method, a stretch molding method, and the like. Among these methods, in order to obtain a film having excellent mechanical strength, surface accuracy, and the like, an extrusion molding method, an inflation molding method, or a press molding method is preferable and an extrusion molding method is most preferable. The molding conditions are appropriately selected depending on the purpose of use and the molding method, and in a case of the heat-melting molding method, the cylinder temperature is appropriately set in a range of usually 150° C. to 400° C., preferably 200° C. to 350° C., and more preferably 230° C. to 330° C. In a case where the polymer temperature is too low, the fluidity deteriorates, which may cause sink marks and distortion in the film, and in a case where the polymer temperature is too high, voids or silver streaks may be generated due to thermal decomposition of the polymer, or molding defects such as yellowing of the film may occur.

(Physical Properties or Characteristics of Optical Filter)

Preferred physical properties or characteristics of the optical filter according to the embodiment of the present invention will be described.

As described above, the optical filter according to the embodiment of the present invention has a small variation in the state of existence of the squarylium compound and the like, and is excellent in surface condition. Specifically, it is as shown in the evaluation of surface condition in Examples described later.

In consideration of handling in a case of laminating and improvement of productivity by shortening the drying time, the thickness of the optical filter is in a range of usually 0.1 to 300 μm, preferably 0.2 to 200 μm, and more preferably 0.3 to 100 μm.

The wetting tension of a surface of the optical filter is preferably 40 mN/m or more, more preferably 50 mN/m or more, and still more preferably 55 mN/m or more. In a case where the wetting tension of the surface is within the above-described range, the adhesive strength between the optical filter and the polarizer is improved. In order to adjust the wetting tension of the surface, a known surface treatment such as a corona discharge treatment, an ozone spraying, an ultraviolet irradiation, a flame treatment, and a chemical treatment can be performed.

The phase difference (retardation) of the optical filter according to the embodiment of the present invention will be described. The in-plane phase difference value Ro at 589 nm of the optical filter according to the embodiment of the present invention is preferably 0 to 20 nm and more preferably 0 to 10 nm. In addition, the phase difference value Rth in the thickness direction is preferably −20 to 50 nm and more preferably −10 to 20 nm.

Generally, the retardation can be controlled by a retardation of the film before stretching, a stretching ratio, a stretching temperature, and a thickness of a stretched alignment film. In a case where the film before stretching has a constant thickness, since the absolute value of retardation tends to increase as the stretching ratio increases, a stretched alignment film having a desired retardation can be obtained by changing the stretching ratio.

In a case where the optical filter is subjected to the stretching treatment, the thickness of the optical filter before stretching is preferably approximately 50 to 500 μm, and it is preferable that the uneven thickness is small, which is within ±8%, preferably within ±6%, and more preferably within ±4% in the entire surface.

The stretching ratio is preferably 1.1 to 10 times and more preferably 1.3 to 8 times, and it is sufficient to set a stretching ratio within the range to be a desired retardation.

In the obtained optical filter as described above, the molecules are aligned by stretching so that the optical filter can have a desired retardation value.

It is preferable that the variation of retardation is small, in which, in the optical filter according to the embodiment of the present invention, the variation of retardation at a wavelength of 589 nm in any retardation of the in-plane direction or the thickness direction is usually within ±50 nm, preferably ±30 nm or less, and more preferably ±20 nm or less.

The variations of retardation in the in-plane direction and the thickness direction or the uneven thickness of the optical filter can be reduced by using a film before stretching which has a smaller variation or uneven thickness, or by applying stress evenly to the film during stretching. For the purpose, it is desirable to stretch the film under an environment in which the temperature is controlled in a uniform temperature distribution, preferably within ±5° C., still more preferably within ±2° C., and particularly preferably within ±0.5° C.

[Image Display Device]

Examples of the image display device according to the embodiment of the present invention include a liquid crystal display device and an organic electroluminescent display device. The image display device according to the embodiment of the present invention will be described using a liquid crystal display device (also referred to as a “liquid crystal display device of the present invention”) as a preferred aspect.

The liquid crystal display device of the present invention has a feature of including at least one optical filter according to the embodiment of the present invention. The optical filter according to the embodiment of the present invention may be used as a polarizing plate-protective film and/or a pressure-sensitive adhesive layer as described later, or may be included in a backlight unit used in a liquid crystal display device.

It is preferable that the liquid crystal display device includes an optical filter, polarizing plates including a polarizer and a polarizing plate-protective film, a pressure-sensitive adhesive layer, and a liquid crystal cell, and it is preferable that the polarizing plates are attached to the liquid crystal cell through the pressure-sensitive adhesive layer. In the liquid crystal display device, the optical filter may also serve as the polarizing plate-protective film or the pressure-sensitive adhesive layer. That is, it is divided into a case where the liquid crystal display device includes polarizing plates including a polarizer and an optical filter (polarizing plate-protective film), a pressure-sensitive adhesive layer, and a liquid crystal cell, and a case where the liquid crystal display device includes polarizing plates including a polarizer and a polarizing plate-protective film, an optical filter (pressure-sensitive adhesive layer), and a liquid crystal cell.

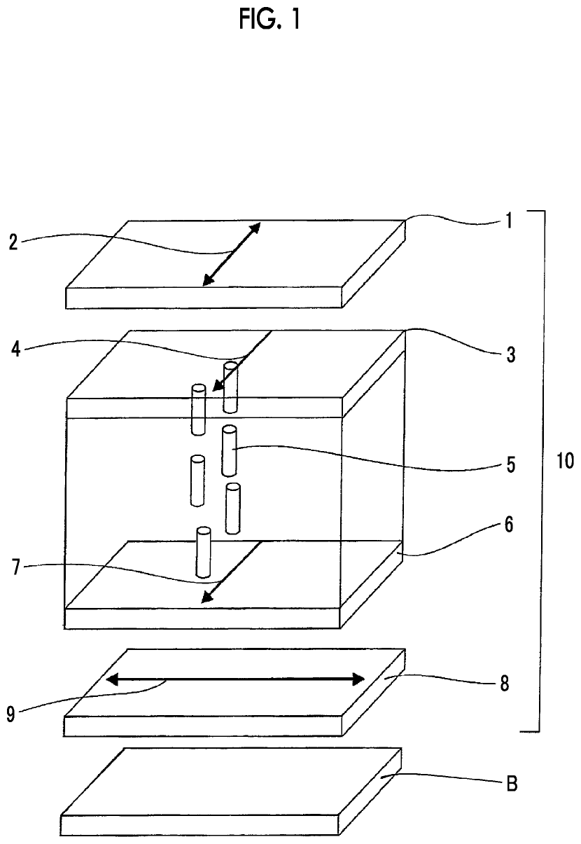

FIG. 1 is a schematic view showing an embodiment of the liquid crystal display device of the present invention. In FIG. 1, a liquid crystal display device 10 consists of a liquid crystal cell having a liquid crystal layer 5 and a liquid crystal cell upper electrode substrate 3 and a liquid crystal cell lower electrode substrate 6 disposed above and below the liquid crystal layer 5, and an upper polarizing plate 1 and a lower polarizing plate 8 disposed on both sides of the liquid crystal cell (directions of the respective absorption axes are indicated by arrows labeled reference numeral 2 or 9). A color filter layer may be laminated on the liquid crystal cell upper electrode substrate 3 or the liquid crystal cell lower electrode substrate 6 (directions of the respective controlled alignments are indicated by arrows labeled reference numeral 4 or 7). On a rear surface of the above-described liquid crystal display device 10, a backlight unit B is disposed. A light source of the backlight unit B is not particularly limited. For example, a light emitting device formed of a white LED can be used.

It is preferable that each of the upper polarizing plate 1 and the lower polarizing plate 8 has a configuration in which two polarizing plate-protective films and a polarizer are laminated so as to sandwich the polarizer with the polarizing plate-protective films, and in the liquid crystal display device 10 of the present invention, at least one polarizing plate is a polarizing plate including the optical filter according to the embodiment of the present invention (not shown).

In addition, in the liquid crystal display device 10 of the present invention, the liquid crystal cell and the polarizing plate (upper polarizing plate 1 and/or lower polarizing plate 8) may be attached together through a pressure-sensitive adhesive layer (not shown). In this case, the optical filter according to the embodiment of the present invention may also serve as the pressure-sensitive adhesive layer.

The liquid crystal display device 10 includes an image direct vision-type liquid crystal display device, an image projection-type liquid crystal display device, and an optical modulation-type liquid crystal display device. An active matrix liquid crystal display device in which a three-terminal or two-terminal semiconductor element such as TFT or MIM is used is effective for the present invention. In addition, a passive matrix liquid crystal display device represented by an STN mode which is called as time division driving is also effective.

In a case where the optical filter according to the embodiment of the present invention is included in the backlight unit B, the polarizing plate of the liquid crystal display device may be a normal polarizing plate (polarizing plate not including the optical filter according to the embodiment of the present invention), or may be a polarizing plate including the optical filter according to the embodiment of the present invention. In addition, the pressure-sensitive adhesive layer may be a normal pressure-sensitive adhesive layer (not the optical filter according to the embodiment of the present invention), or may be a pressure-sensitive adhesive layer formed of the optical filter according to the embodiment of the present invention.

An IPS mode liquid crystal display device described in paragraphs 0128 to 0136 of JP2010-102296A is preferable as the liquid crystal display device of the present invention.

<Polarizing Plate>

The polarizing plate used in the present invention includes a polarizer and at least one polarizing plate-protective film.

The polarizing plate used in the present invention is preferably a polarizing plate having a polarizer and polarizing plate-protective films on both surfaces of the polarizer, and it is preferable that at least one surface of the polarizer includes the optical filter according to the embodiment of the present invention as the polarizing plate-protective film. The opposite surface of the polarizer to the surface having the optical filter according to the embodiment of the present invention (polarizing plate-protective film) may have a normal polarizing plate-protective film.

The film thickness of the polarizing plate-protective film used in the present invention is 5 μm to 120 μm and more preferably 10 μm to 100 μm. A thinner film is preferable in that, in a case of being incorporated in the liquid crystal display device, display unevenness over time in high temperature and high humidity is less likely to occur. On the other hand, in a case where the film is too thin, it is difficult to handle the film stably in a case of manufacturing the film and producing the polarizing plate. It is preferable that the thickness of the optical filter constituting the polarizing plate-protective film satisfies the above-described range.

—Shape and Configuration—

The shape of the polarizing plate used in the present invention includes not only a polarizing plate of an aspect of a film piece cut into a size so as to be incorporated in the liquid crystal display device as it is, but also a polarizing plate of an aspect in which the polarizing plate is produced in a longitudinal shape by a continuous production and wound up in a rolled shape (for example, an aspect having a roll length of 2500 μm or more or 3900 μm or more). In order to use the polarizing plate as a large-sized screen liquid crystal display device, the width of the polarizing plate is preferably 1470 mm or more.

The polarizing plate used in the present invention is configured of a polarizer and at least one polarizing plate-protective film, but it is also preferable that the polarizing plate is further configured by attaching a separate film on one surface of the polarizing plate.

The separate film is used for the purpose of protecting the polarizing plate during the shipping of the polarizing plate and the inspection of product. The separate film is used for the purpose of covering an adhesive layer which is attached to a liquid crystal plate, and used on a surface where the polarizing plate is attached to the liquid crystal plate.

(Polarizer)

The polarizer used for the polarizing plate used in the present invention will be described.

The polarizer which can be used for the polarizing plate used in the present invention is preferably configured of polyvinyl alcohol (PVA) and a dichroic molecule, but as described in JP1999-248937A (JP-H11-248937A), a polyvinylene-based polarizer in which a polyene structure is generated by dehydrating PVA or dechlorinating polyvinyl chloride and aligning the polyene structure can also be used.

—Film Thickness of Polarizer—

The film thickness of the polarizer before stretching is not particularly limited, but from the viewpoint of stability of retaining film and homogeneity of stretching, is preferably 1 μm to 1 mm and particularly preferably 5 to 200 μm. In addition, as described in JP2002-236212A, a thin PVA film in which the stress generated in a case of being stretched 4 to 6 times in water is 10 N or less may be used.

—Method of Manufacturing Polarizer—

The method of manufacturing a polarizer is not particularly limited, and for example, it is preferable that the polarizer is configured by form PVA into a film and introducing the dichroic molecule to the film. The PVA film can be manufactured by the method described in paragraphs “0213” to “0237” of JP2007-86748A and by the description of—JP3342516B, JP1997-328593A (JP-H09-328593A), JP2001-302817A, JP2002-144401A, and the like.

(Method of Laminating Polarizer and Polarizing Plate-Protective Film)

The polarizing plate used in the present invention is manufactured by adhering (laminating) at least one polarizing plate-protective film (preferably the optical filter according to the embodiment of the present invention) on at least one surface of the above-described polarizer.

The polarizing plate used in the present invention is preferably produced by a method in which a polarizing plate-protective film is subjected to an alkali treatment, and is attached, using a completely saponified polyvinyl alcohol aqueous solution, to both surfaces of a polarizer produced by immersing and stretching a polyvinyl alcohol film in an iodine solution.

Examples of an adhesive used to attach the treated surface of the polarizing plate-protective film to the polarizer include polyvinyl alcohol-based adhesives such as polyvinyl alcohol and polyvinyl butyral and vinyl-based latex such as butyl acrylate.

In the polarizing plate used in the present invention, a method of attaching the polarizing plate-protective film to the polarizer is preferably a method in which the polarizing plate-protective film is attached to the polarizer so that a transmission axis of the polarizer and a slow axis of the polarizing plate-protective film are substantially parallel, orthogonal, or 45°.

The slow axis can be measured by various known methods, for example, using a birefringence meter (KOBRADH, manufactured by Oji Scientific Instruments).

Here, “substantially parallel” refers to that the direction of the main refractive index nx of the polarizing plate-protective film and the direction of the transmission axis of the polarizing plate intersect at an angle within ±5°, preferably at an angle within ±1, and more preferably angle within ±0.5°. In a case where the intersecting angle is within 1°, polarization performance under polarizing plate crossed nicols is less likely to be deteriorated and light leakage does not easily occur, which is preferable.

The description in which the direction of the main refractive index nx and the direction of the transmission axis are orthogonal or 45° means that the angle at which the direction of the main refractive index nx and the direction of the transmission axis intersect is within a range of ±5° with respect to an exact angle of being orthogonal and 45°, and the difference with respect to the exact angle is preferably within a range of ±1° and more preferably within a range of ±0.5°.

(Functionalization of Polarizing Plate)

The polarizing plate used in the present invention is preferably used as a functionalized polarizing plate complexed with an antireflection film for improving visibility of a display, a luminance improving film, or an optical film having a functional layer such as a hard coat layer, a forward scattering layer, an antiglare layer, an antifouling layer, and an antistatic layer. The antireflection film for functionalization, the luminance improving film, other functional optical films, the hard coat layer, the forward scattering layer, and the antiglare layer are described in paragraphs “0257” to “0276” of JP2007-86748A, and a functionalized polarizing plate can be produced based on the description.

(Pressure-Sensitive Adhesive Layer)

In the liquid crystal display device of the present invention, the polarizing plate is preferably attached to the liquid crystal cell through a pressure-sensitive adhesive layer. The optical filter according to the embodiment of the present invention may also serve as the pressure-sensitive adhesive layer. In a case where the optical filter according to the embodiment of the present invention does not serve as the pressure-sensitive adhesive layer, a normal pressure-sensitive adhesive layer can be used as the pressure-sensitive adhesive layer.

The pressure-sensitive adhesive layer is not particularly limited as long as the pressure-sensitive adhesive layer can attach the polarizing plate to the liquid crystal cell, and for example, an acrylic type, a urethane type, polyisobutylene, or the like is preferable.

In a case where the optical filter according to the embodiment of the present invention also serves as a pressure-sensitive adhesive layer, the pressure-sensitive adhesive layer includes the coloring agent and the binder, and further contains a cross-linking agent, a coupling agent, or the like to impart adhesiveness.

In a case where the optical filter also serves as a pressure-sensitive adhesive layer, the pressure-sensitive adhesive layer includes the binder in an amount of preferably 90% to 100% by mass and preferably 95% to 100% by mass. The content of the coloring agent is as described above.

The thickness of the pressure-sensitive adhesive layer is not particularly limited, but is preferably 1 to 50 μm and more preferably 3 to 30 μm.

(Liquid Crystal Cell)

The liquid crystal cell is not particularly limited, and a normal liquid crystal cell can be used.

[Solid-State Imaging Element]

The solid-state imaging element according to the embodiment of the present invention includes the optical filter according to the embodiment of the present invention. The configuration of the solid-state imaging element according to the embodiment of the present invention is not particularly limited as long as the solid-state imaging element includes the optical filter according to the embodiment of the present invention and functions as a solid-state imaging element. Since the solid-state imaging element according to the embodiment of the present invention includes the optical filter (color filter) according to the embodiment of the present invention having excellent weather fastness and contrast, the solid-state imaging element according to the embodiment of the present invention is excellent in image tone and color reproducibility over a long period of use.

The configuration of the solid-state imaging element is not particularly limited as long as the solid-state imaging element includes the color filter of the present invention and functions as a solid-state imaging element. Examples thereof include a configuration in which, on a support, a solid-state imaging element (CCD image sensor, CMOS image sensor, or the like) has light-receiving elements which consist of a plurality of photodiodes and polysilicon or the like and constitute a light-receiving area of the solid-state imaging element, and the color filter of the present invention is provided on forming surface of the light-receiving elements in the support (for example, a portion other than a light receiving section, a pixel section for color adjustment, or the like) or on the opposite side of the forming surface.

### EXAMPLES

Hereinafter, the present invention will be described in detail using examples. The materials, reagents, amounts and proportions of substances, operations, and the like described in the following examples can be appropriately modified as long as the gist of the present invention is maintained. Therefore, the scope of the present invention is not limited to the specific examples described below.

In the present invention, “room temperature” means 25° C.

### [Example A] Synthesis and Evaluation of Squarylium Compound

### [Synthesis Example 1] Synthesis of Compound B-12

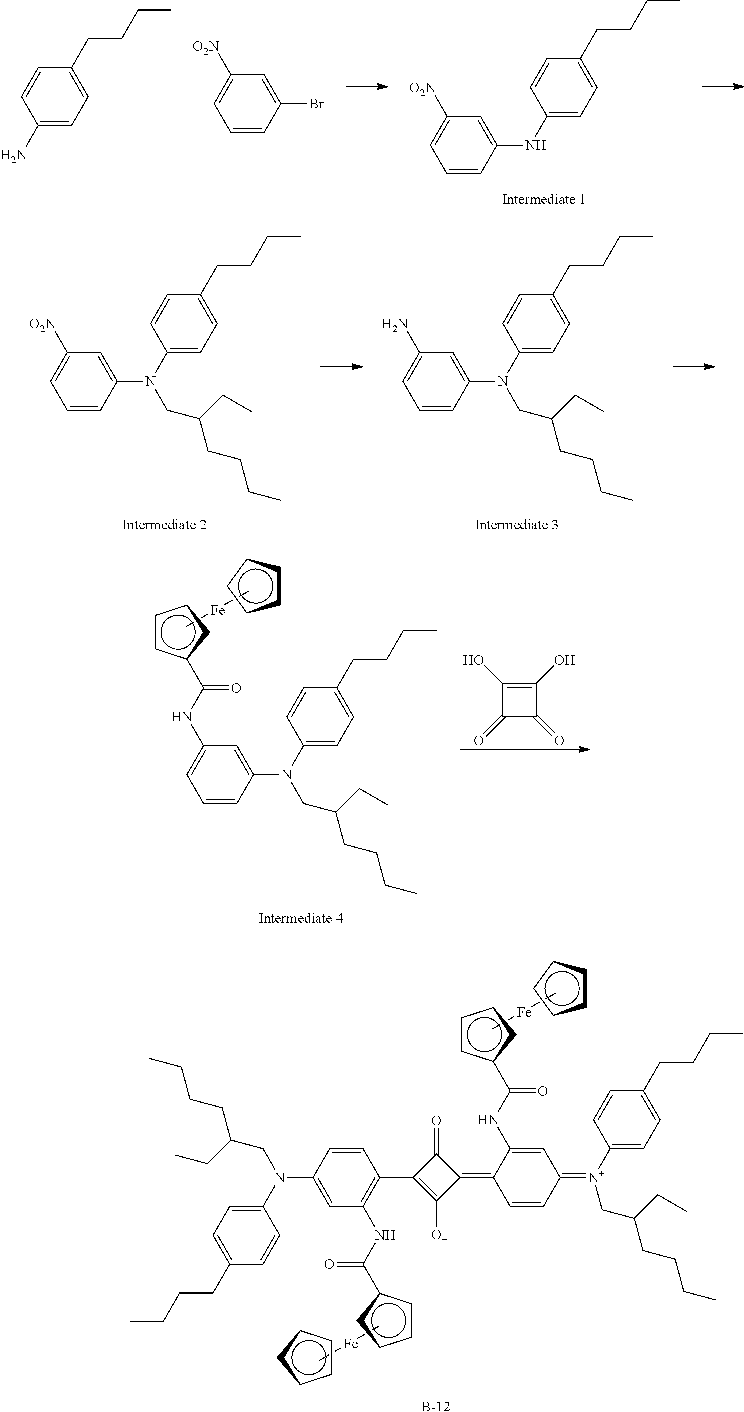

0.91 g of 4-butylaniline, 1.00 g of 3-bromonitrobenzene, 1.85 g of potassium carbonate, and 30 mL of isopropanol were mixed with each other, and then the mixture was stirred at room temperature for 1 hour while performing nitrogen bubbling. Subsequently, 0.16 g of 2-dicyclohexylphosphino-2′,4′,6-triisopropylbiphenyl (xPhos) and 0.061 g of tris(dibenzylideneacetone) dipalladium (Pd₂bda₃) were added thereto, and the reaction mixture was heated and stirred at 110° C. for 8 hours. After completion of the reaction, the reaction mixture was returned to room temperature and then ice-cooled, 30 mL of water in which 2.15 g of ammonium chloride was dissolved was slowly added dropwise thereto, and the mixture was further stirred for 1 hour. The obtained crystals were filtered, and the obtained filtrates (crystals) were purified by silica gel column chromatography to obtain 0.65 g (54%) of an intermediate 1.

40 mL of dimethylacetamide was added to 4.5 g of the intermediate 1, 0.7 g of sodium hydride was slowly added thereto while stirring under ice-cooling, and the mixture was stirred for 30 minutes. Subsequently, 4.25 g of 2-ethylhexyl bromide was added dropwise thereto, and the reaction mixture was heated and stirred at room temperature for 6 hours and at an internal temperature of 45° C. for 6 hours. After completion of the reaction, the reaction mixture was cooled, and 100 mL of water was added dropwise thereto. Subsequently, 100 mL of ethyl acetate and 100 mL of hexane were added thereto to extract an organic layer. The organic layer was washed with water and saturated saline, and the obtained organic layer was dried and concentrated over magnesium sulfate, and then purified by silica gel column chromatography (hexane/ethyl acetate=4/1) to obtain 2.0 g (31%) of an intermediate 2.

20 mL of tetrahydrofuran was added to 2.0 g of the intermediate 2 and stirred, and then 1.0 g of palladium hydroxide was added. Subsequently, the inside of the flask was sufficiently replaced with hydrogen gas, and then the reaction was carried out at room temperature for 5 hours. After completion of the reaction, the reaction mixture was filtered through Celite, and the obtained filtrate was concentrated and purified by silica gel column chromatography (hexane/ethyl acetate=4/1) to obtain 1.8 g (61%) of an intermediate 3.

15 mL of dimethylacetamide was added to 1.1 g of the intermediate 3, 0.42 g of dimethylaminopyridine and 0.72 g of ferrocenecarboxylic acid were further added thereto, and the mixture was stirred at room temperature for 30 minutes. Subsequently, 0.66 g of 1-(3-dimethylaminopropyl)-3-ethylcarbodiimide hydrochloride was added to the obtained mixture, and the reaction mixture was stirred at room temperature for 24 hours. After completion of the reaction, 50 mL of hexane, ethyl acetate, and 100 mL of 1N hydrochloric acid were added to the reaction mixture to extract an organic layer. The organic layer was washed with water and saturated saline, and the obtained organic layer was dried and concentrated over magnesium sulfate, and then purified by silica gel column chromatography (hexane/ethyl acetate=4/1) to obtain 1.8 g (88%) of an intermediate 4.

0.80 g of the intermediate 4, 0.08 g of squaric acid, 10 mL of toluene, and 10 mL of n-butanol were put into a flask equipped with a Dean Stark tube, mixed, and heated and refluxed for 4 hours. After completion of the reaction, the reaction mixture was cooled to 0° C., the obtained crystals were filtered, and the filtrate (crystal) was washed with methanol. 10 mL of methanol was added to the obtained crude crystal, and the mixture was further heated and refluxed for 1 hour, the obtained crystal was filtered, and the obtained filtrate (crystal) was washed with methanol. As a result, 0.69 g (80%) of a squarylium compound B-12 was obtained.

The obtained squarylium compound B-12 was identified by a nuclear magnetic resonance spectrum (¹H-NMR).

¹H-NMR (CDCl₃): δ 11.77 to 11.33 (m, 2H), 8.50 to 8.22 (m, 4H), 7.28 to 7.26 (m, 4H), 7.15 to 7.13 (m, 4H), 6.36 to 6.33 (m, 2H), 5.32 to 5.20 (m, 4H), 4.44 to 4.34 (m, 4H), 4.23 to 4.15 (m, 10H), 3.84 to 3.74 (m, 4H), 2.69 to 2.65 (m, 4H), 1.85 to 1.80 (m, 2H), 1.69 to 1.62 (m, 4H), 1.51 to 1.36 (m, 12H), 1.24 to 1.22 (m, 8H), 0.97 (t, 6H), 0.87 to 0.83 (m, 12H)

### [Test Example 1] Measurement of Maximal Absorption Wavelength of Squarylium Compound

The obtained squarylium compound B-12 was dissolved in chloroform (concentration: 1×10⁻⁶ mol/L), and the maximum absorption wavelength λmax of the squarylium compound B-12 was measured using a cell having an optical path length of 10 mm and using a spectrophotometer UV-1800PC (manufactured by Shimadzu Corporation). The measurement result of the maximum absorption wavelength λmax of the compound B-12 is shown in Table 1 below.

### [Test Example 2] Evaluation of Solubility of Squarylium Compound

Solubility of the obtained squarylium compound B-12 in a mixed solvent of toluene/cyclohexanone (toluene/cyclohexanone=90/10 (vol %)) was confirmed. Specifically, a dissolved amount (% by mass) of squarylium compound B-12 dissolved in 100 parts by mass of the mixed solvent of toluene/cyclohexanone was measured.

The solubility was evaluated by applying the obtained dissolved amount to the following standard.

—Evaluation Standard of Solubility—

A: 0.1% by mass or more

B: 0.01% by mass or more and less than 0.1% by mass

C: less than 0.01% by mass

Squarylium compounds shown in Table 1 below and comparative compounds C-1 to C-6 were synthesized according to [Synthesis Example 1] described above, respectively.

Hereinafter, specific methods for synthesizing compounds A-19, A-28, and A-4 will be shown.

### [Synthesis Example 2] Synthesis of Compound A-19

A compound A-19 was synthesized according to the following scheme.

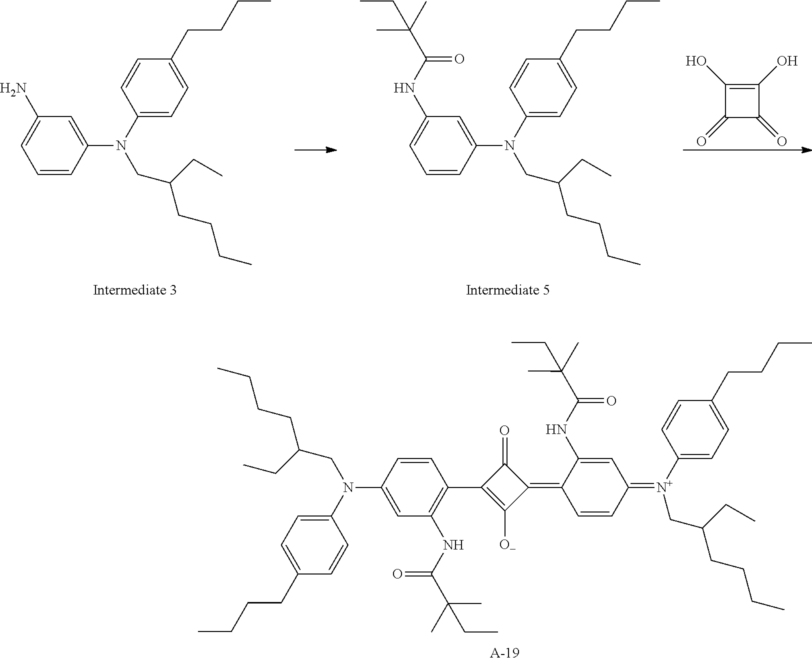

13 mL of dimethylacetamide was added to 2.1 g of the intermediate 3 obtained in [Synthesis Example 1] described above and stirred under ice-cooling, and then 0.92 g of 2,2-dimethylbutyryl chloride was slowly added dropwise thereto. After completion of the dropwise addition, the reaction mixture was returned to room temperature and stirred at room temperature for 4 hours. After completion of the reaction, the mixture was ice-cooled again, 40 mL of water was added dropwise thereto, and a 5% NaOH aqueous solution was further added thereto until a pH of the reaction solution reached 8. Thereafter, 60 mL of ethyl acetate was added thereto to extract an organic layer. The organic layer was washed with water and saturated saline, and the obtained organic layer was dried and concentrated over magnesium sulfate, and then purified by silica gel column chromatography (hexane/ethyl acetate=8/1) to obtain 2.4 g (86%) of an intermediate 5.

2.2 g of the intermediate 5, 0.42 g of squaric acid, 10 mL of toluene, and 10 mL of n-butanol were put into a flask equipped with a Dean Stark tube, mixed, and heated and refluxed for 10 hours. After completion of the reaction, the reaction mixture was cooled to room temperature, 30 mL of methanol was added thereto, and the mixture was stirred at room temperature for 2 hours. The obtained crystal was filtered, and the filtrate (crystal) was washed with methanol. As a result, 2.0 g (85%) of a squarylium compound A-19 was obtained.

The obtained squarylium compound A-19 was identified by a nuclear magnetic resonance spectrum (¹H-NMR).

¹H-NMR (CDCl₃): δ 11.37 to 11.05 (m, 2H), 8.48 to 8.41 (m, 4H), 7.26 to 7.24 (d, 4H), 7.11 to 7.09 (d, 4H), 6.26 to 6.24 (d, 2H), 3.76 to 3.74 (m, 4H), 2.67 to 2.63 (m, 4H), 1.83 to 1.74 (m, 6H), 1.67 to 1.60 (m, 4H), 1.41 to 1.33 (m, 24H), 1.25 to 1.21 (m, 8H), 0.97 to 0.93 (m, 6H), 0.89 to 0.81 (m, 18H)

### [Synthesis Example 3] Synthesis of Compound A-28

A compound A-28 was synthesized according to the following scheme.

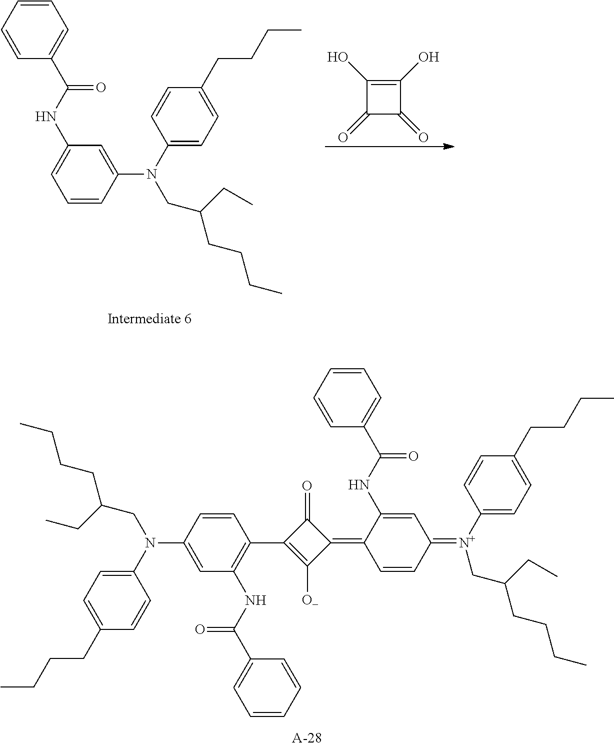

2.2 g of the intermediate 6, 0.42 g of squaric acid, 10 mL of toluene, and 10 mL of n-butanol were put into a flask equipped with a Dean Stark tube, mixed, and heated and refluxed for 10 hours. After completion of the reaction, the reaction mixture was cooled to room temperature, 30 mL of methanol was added thereto, and the mixture was stirred at room temperature for 2 hours. The obtained crystal was filtered, and the filtrate (crystal) was washed with methanol. The obtained crude crystal was further purified by silica gel column chromatography (hexane/ethyl acetate=6/1) and then dried. As a result, 1.6 g (67%) of a squarylium compound A-28 was obtained.

The intermediate 6 was synthesized with reference to the synthesis method of the intermediate 5 in [Synthesis Example 2].

The obtained squarylium compound A-28 was identified by a nuclear magnetic resonance spectrum (¹H-NMR).

¹H-NMR (CDCl₃): δ 12.40 to 12.03 (m, 2H), 8.44 to 8.34 (m, 4H), 8.12 to 7.90 (m, 4H), 7.65 to 7.39 (m, 4H), 7.37 to 7.27 (m, 5H), 7.13 to 7.03 (m, 5H), 6.35 to 6.32 (m, 2H), 3.85 to 3.75 (m, 4H), 2.68 to 2.64 (m, 4H), 1.85 to 1.79 (m, 2H), 1.68 to 1.61 (m, 4H), 1.51 to 1.33 (m, 12H), 1.25 to 1.21 (m, 8H), 0.98 to 0.94 (m, 6H), 0.87 to 0.81 (m, 12H)

### [Synthesis Example 4] Synthesis of Compound A-4

A compound A-4 was synthesized according to the following scheme.

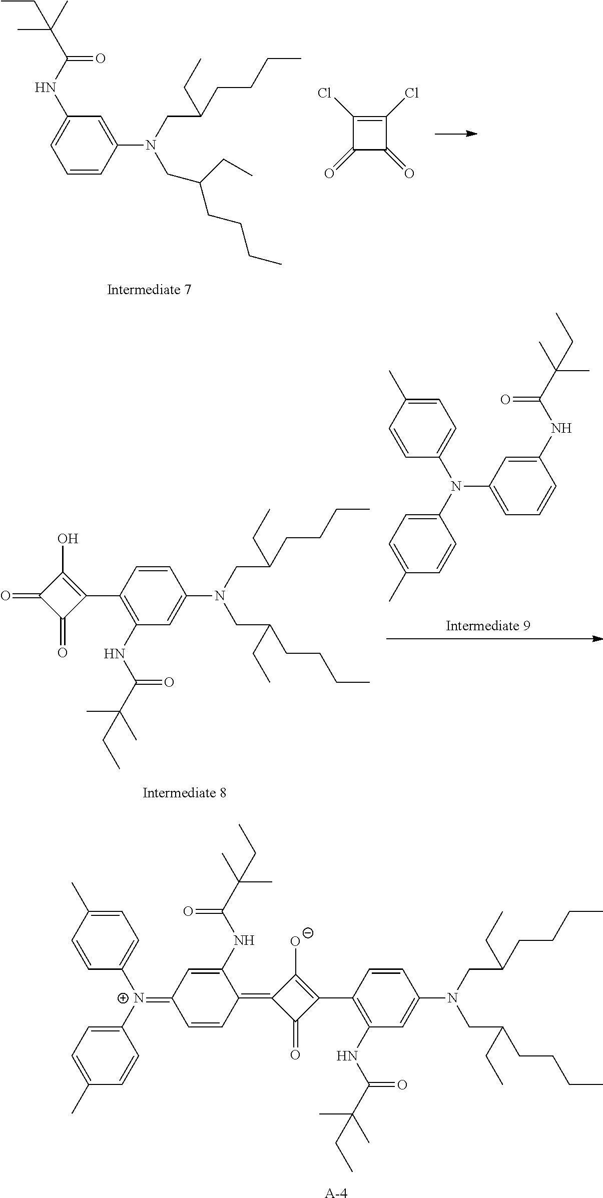

50 mL of toluene was added to 5.7 g of an intermediate 7 and 2.0 g of squaric acid dichloride, and the reaction mixture was heated and refluxed for 8 hours. After completion of the reaction, the reaction mixture was cooled to room temperature, 100 mL of water was added dropwise to the reaction mixture and stirred for 1 hour, and 50 mL of toluene was added thereto to extract an organic layer. The organic layer was repeatedly washed with water, and then the organic layer was concentrated under reduced pressure. Subsequently, 50 mL of acetic acid, 50 mL of water, and 4 mL of 2N hydrochloric acid water were added to the concentrated residue, and the mixture was heated and refluxed for 8 hours. After completion of the reaction, the reaction mixture was cooled to room temperature, the reaction solvent was concentrated under reduced pressure, and 10 mL of methanol was added thereto. After stirring at room temperature for 1 hour, the obtained crystal was filtered, and the filtrate (crystal) was washed with water and methanol, and then dried. In this way, 3.1 g (45%) of a target intermediate 8 was obtained.

1.0 g of the intermediate 8, 0.73 g of the intermediate 9, 10 mL of toluene, and 10 mL of n-butanol were put into a flask equipped with a Dean Stark tube, mixed, and heated and refluxed for 2 hours. After completion of the reaction, the reaction mixture was cooled to 0° C., the obtained crystals were filtered, and the filtrate (crystal) was washed with methanol. The obtained crude crystal was further purified by silica gel column chromatography (hexane/ethyl acetate=2/1) and then dried. In this way, 1.1 g (68%) of a target compound A-4 was obtained.

The intermediate 7 and the intermediate 9 were synthesized with reference to the synthesis method of the intermediate 5 in [Synthesis Example 2], respectively.

The obtained squarylium compound A-4 was identified by matrix-assisted laser desorption/ionization mass spectrometry (MALDI-MS).

MS: m/z=895.6 ([M+H]⁺)

With regard to each of the synthesized compounds, the measurement of the maximum absorption wavelength λmax and the evaluation of the solubility were carried out in the same manner as in Test Example 1 and Test Example 2 described above, and the results thereof are shown in Table 1. For the comparative compounds C-1 to C-3, C-5, and C-6, the measured dissolved amounts are also described.

In Table 1, the numbers assigned to each squarylium compound correspond to Exemplary Compound numbers of the squarylium compounds described above (the same applies to Tables 2 to 5).

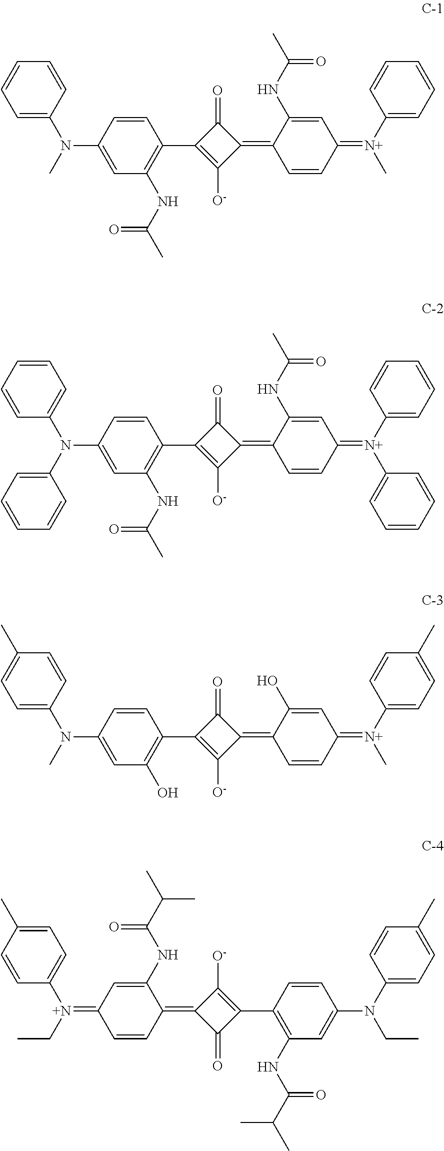 [SMILES: CC(=O)NC1=CC(=[N+](/C)c2ccccc2)/C=CC/1=C1/C(=O)C(c2ccc(N(C)c3ccccc3)cc2NC(C)=O)=C1[O-] ; CC(=O)NC1=CC(=[N+](c2ccccc2)c2ccccc2)C=C/C1=C1/C(=O)C(c2ccc(N(c3ccccc3)c3ccccc3)cc2NC(C)=O)=C1[O-] ; Cc1ccc(N(C)c2ccc(C3=C([O-])/C(=C4C=C/C(=[N+](\C)c5ccc(C)cc5)C=C\4O)C3=O)c(O)c2)cc1 ; CCN(c1ccc(C)cc1)c1ccc(C2=C([O-])/C(=C3C=C/C(=[N+](\CC)c4ccc(C)cc4)C=C/3NC(=O)C(C)C)C2=O)c(NC(=O)C(C)C)c1]

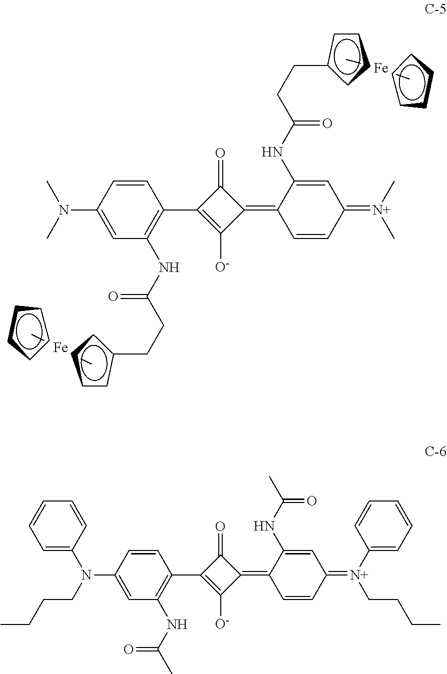

TABLE 1

| Squarylium | λmax | |
|---|---|---|
| compound | (nm) | Solubility |
| A-1 | 688 | A |
| A-2 | 690 | A |
| A-4 | 699 | A |
| A-5 | 681 | A |
| A-8 | 693 | A |
| A-10 | 691 | A |
| A-12 | 687 | A |
| A-14 | 693 | A |
| A-16 | 688 | A |
| A-19 | 687 | A |
| A-20 | 696 | A |
| A-21 | 688 | A |
| A-22 | 688 | A |
| A-24 | 684 | A |
| A-26 | 688 | A |
| A-28 | 695 | A |
| A-29 | 695 | A |
| A-30 | 697 | A |
| B-1 | 700 | B |
| B-2 | 691 | A |
| B-3 | 684 | A |
| B-4 | 714 | A |
| B-5 | 693 | B |
| B-7 | 694 | A |
| B-11 | 697 | A |
| B-12 | 696 | A |
| B-15 | 691 | B |
| B-20 | 701 | A |
| C-1 | 680 | C |
| | | (<0.005% |
| | | by mass) |
| C-2 | 737 | C |
| | | (<0.005% |
| | | by mass) |
| C-3 | 658 | C |
| | | (<0.005% |
| | | by mass) |
| C-4 | 685 | B |
| C-5 | 682 | C |
| | | (<0.005% |
| | | by mass) |
| C-6 | 681 | C |
| | | (<0.01% |
| | | by mass) |

### [Example B] Preparation of Resin Composition and Production and Evaluation of Optical Filter

The resin composition (liquid composition) according to the embodiment of the present invention was prepared using the squarylium compound synthesized in Example A, and an optical filter was produced and evaluated for light resistance and surface condition.

Materials used in this example are shown below.

(Resin 1)

A commercially available polystyrene (manufactured by PS Japan Corporation, SGP-10, Tg: 100° C.) was heated at 110° C., returned to normal temperature (23° C.), and then used.

(Resin 2)

A commercially available Arton (manufactured by JSR Corporation, RX4500, Tg: 140° C., cyclic polyolefin) was heated at 110° C., returned to normal temperature, and then used.

(Base Material Film 1)

A commercially available polyethylene terephthalate film, LUMIRROR® S105 (film thickness: 38 μm, manufactured by Toray Industries, Inc.) was used as a base material 1.

### Example 28

(Preparation of Resin Composition)

The following components were mixed (dissolved in a toluene/cyclohexanone mixed solvent) to prepare a resin solution S-1 as one form of the resin composition according to the embodiment of the present invention.

Composition of resin solution S-1

| | | |
|---|---|---|
| Resin 1 | 100 | parts by mass |
| Squarylium compound B-12 | 1.49 | parts by mass |
| Toluene (solvent) | 1710 | parts by mass |
| Cyclohexanone (solvent) | 190 | parts by mass |

Next, the obtained resin solution S-1 was filtered using a filter paper having an absolute filtration precision of 10 μm (#63, manufactured by Toyo Roshi Kaisha, Ltd.) and further filtered using a metal-sintered filter having an absolute filtration precision of 2.5 μm (FH025, manufactured by Pall Corporation).

(Production of Optical Filter)

The resin solution S-1 after the above-described filtration treatment was applied onto the base material film 1 using a bar coater so that a film thickness after drying was 5.0 μm, and dried at 100° C., thereby producing an optical filter (resin film) 101 as a coated and dried product.

### <Test Example 3> Evaluation of Light Resistance of Optical Filter

Light resistance of the optical filter 101 produced in Example 28 was evaluated by a rate of change in absorbance (%).

Specifically, using Super xenon weather meter SX75 (trade name, manufactured by Suga Test Instruments Co., Ltd.), the optical filter 101 was irradiated with light at 100,000 lux for 90 hours under an environment of 50° C. and a relative humidity of 50%, the difference in absorbance at the absorption maximal wavelength was measured, and then the rate of change in absorbance was calculated by the following expression. The results are shown in Table 2.

(Rate of change in absorbance) (%)=[(difference in absorbance after 90 hour irradiation)/(difference in absorbance before 90 hour irradiation)]×100

Here, the difference in absorbance of the optical filter at the absorption maximal wavelength was determined as follows.

Using a spectrophotometer UV3600 (manufactured by Shimadzu Corporation), with regard to the optical filter 101 and a filter (blank) which was produced in the same manner as the optical filter 101, except that the squarylium compound B-12 was not contained, an absorbance in a wavelength range of 400 to 800 nm was measured every 1 nm. The difference in absorbance between the absorbance of the optical filter 101 and the absorbance of the filter (blank) at each wavelength was calculated, and a wavelength at which the difference in absorbance was maximum was defined as the absorption maximal wavelength. That is, the maximum difference in absorbance was defined as the difference in absorbance of the optical filter 101 at the absorption maximal wavelength.

### <Test Example 4> Evaluation of Surface Condition of Optical Filter

A surface condition of the optical filter 101 produced in Example 28 was evaluated by visual observation using an optical microscope. Specifically, using an optical microscope MX-61L (trade name, manufactured by Olympus Corporation), the optical filter 101 was observed at any 10 points with a bright field of 200 times. At each observation point, the presence or absence of unevenness (linear scratches on the surface, irregularities such as protrusions, uneven distribution or associate of squarylium compounds in the film or on the film surface, and the like) in the resin film was confirmed. Specifically, a case where linear scratches on the surface, irregularities caused by cissing, scattering or turbidity of the resin film caused by precipitate due to uneven distribution or associate formation of the squarylium compound can be visually recognized was determined that there was unevenness.

Among all 10 observation points, the total number of observation points obtained by totaling the number of observation points in which the unevenness was not confirmed and the film was uniform was adopted to the following evaluation standard to evaluate the surface condition. The results are shown in Table 2.

—Evaluation Standard of Surface Condition—

A: total number of observation points was 9 or more.

B: total number of observation points was 6 or more and 8 or less.

C: total number of observation points was less than 6.

### Examples 1 to 15, 21 to 27, and 29 to 34 and Comparative Examples 1 to 6

Resin compositions of Examples 1 to 15, 21 to 27, and 29 to 34, and Comparative Examples 1 to 6 were prepared and optical filters thereof were produced in the same manner as in Example 28, except that the resin, the squarylium compound, and the contents thereof in Example 28 were changed to those shown in Table 2. The thickness of each optical filter was also set to be the same as the thickness of the optical filter 101 of Example 28.

In the preparation of the resin composition of Examples 11 to 13, 15, and 31 to 34 in which the resin 2 was used, the toluene/cyclohexanone mixed solvent was changed to a mixed solvent of 1427 parts by mass of cyclohexane and 250 parts by mass of ethyl acetate, and in the production of the optical filter, the base material film 1 was changed to a triacetyl cellulose film ZRD40SL (trade name, manufactured by Fujifilm Corporation).

In addition, since the comparative compounds C-1 to C-6 were not completely dissolved in the toluene/cyclohexanone mixed solvent at the following contents, the optical filter was produced using a resin solution obtained by filtering the insoluble matter.

With regard to each of the produced optical filters, the light resistance and the surface condition were evaluated in the same manner as in Test Example 3 and Test Example 4 described above, and the results thereof are shown in Table 2.

TABLE 2

| | | | | Light | |
|---|---|---|---|---|---|
| | | Squarylium compound | | resistance | Surface |
| | Resin | Type | Content* | (%) | condition |
| Example 1 | Resin 1 | A-1 | 1.02 | 75 | A |
| Example 2 | Resin 1 | A-2 | 2.51 | 70 | A |
| Example 3 | Resin 1 | A-4 | 1.11 | 72 | A |
| Example 4 | Resin 1 | A-5 | 3.11 | 70 | A |
| Example 5 | Resin 1 | A-8 | 0.51 | 76 | A |
| Example 6 | Resin 1 | A-10 | 0.08 | 77 | A |
| Example 7 | Resin 1 | A-12 | 0.89 | 75 | A |
| Example 8 | Resin 1 | A-14 | 1.21 | 74 | A |
| Example 9 | Resin 1 | A-16 | 1.51 | 73 | A |
| Example 10 | Resin 1 | A-19 | 2.41 | 77 | A |
| Example 11 | Resin 2 | A-4 | 3.21 | 74 | A |
| Example 12 | Resin 2 | A-12 | 2.22 | 76 | A |
| Example 13 | Resin 2 | A-19 | 1.42 | 76 | A |
| Example 14 | Resin 1 | A-28 | 1.62 | 77 | A |
| Example 15 | Resin 2 | A-28 | 2.51 | 77 | A |
| Example 21 | Resin 1 | B-1 | 0.12 | 94 | B |
| Example 22 | Resin 1 | B-2 | 1.62 | 91 | A |
| Example 23 | Resin 1 | B-3 | 0.42 | 96 | A |
| Example 24 | Resin 1 | B-4 | 1.31 | 92 | A |
| Example 25 | Resin 1 | B-5 | 0.06 | 87 | B |
| Example 26 | Resin 1 | B-7 | 1.51 | 94 | A |
| Example 27 | Resin 1 | B-11 | 2.85 | 95 | A |
| Example 28 | Resin 1 | B-12 | 1.49 | 96 | A |
| Example 29 | Resin 1 | B-15 | 0.24 | 85 | B |
| Example 30 | Resin 1 | B-20 | 1.51 | 94 | A |
| Example 31 | Resin 2 | B-12 | 0.75 | 97 | A |
| Example 32 | Resin 2 | B-2 | 3.22 | 92 | A |
| Example 33 | Resin 2 | B-11 | 0.91 | 96 | A |
| Example 34 | Resin 2 | B-20 | 2.15 | 95 | A |
| Comparative | Resin 1 | C-1 | 0.69 | 48 | C |
| Example 1 | | | | | |
| Comparative | Resin 1 | C-2 | 0.54 | 38 | C |
| Example 2 | | | | | |
| Comparative | Resin 1 | C-3 | 0.62 | 36 | C |
| Example 3 | | | | | |
| Comparative | Resin 1 | C-4 | 0.83 | 66 | B (6 |
| Example 4 | | | | | points) |
| Comparative | Resin 1 | C-5 | 0.06 | 83 | C (3 |
| Example 5 | | | | | points) |
| Comparative | Resin 1 | C-6 | 0.07 | 55 | B (6 |
| Example 6 | | | | | points) |

In Table 2, “Content (%)” of the squarylium compound is a mass ratio (part by mass) with respect to 100 parts by mass of the resin.

In Examples 1 to 15, 21 to 27, and 29 to 34, and Comparative Examples 1 to 6, resin compositions were prepared and optical filters thereof were produced in the same manner as in each example or comparative example, except that the content of the squarylium compound was changed to 1.49 parts by mass as in Example 28 (resin solution S-1).

As a result, in each of Examples 1 to 15, 21 to 27, and 29 to 34, and Comparative Examples 1 to 6, although the measurement results of light resistance slightly varied from the values shown in Table 2, the values were almost the same as the values shown in Table 2, and the same tendency of improvement in light resistance was confirmed. In addition, the same results as those shown in Table 2 were obtained for the surface condition of the optical filter. As described above, it was found that the same effects could be obtained even in a case where the contents of the squarylium compound in the resin composition and the optical filter were appropriately changed within the ranges specified in the present invention.

### [Example C] Preparation of Resin Composition and Production and Evaluation of Optical Filter

The resin composition (liquid composition) according to the embodiment of the present invention prepared using the squarylium compound synthesized in Example A and a poly(meth)acrylic resin as a resin 3 was applied and dried to produce an optical filter as a coated and dried product, and the light resistance and surface condition of the obtained optical filter were evaluated.

### Example 107

0.07 parts by mass of the squarylium compound B-12, 14 parts by mass of a propylene glycol monomethyl ether acetate solution containing 40% by mass of a resin 3: benzyl methacrylate/methacrylic acid copolymer (molar ratio=70/30, Tg=80° C. to 90° C.), and 30 parts by mass of tetrahydrofuran were mixed with each other to dissolve the squarylium compound and the resin 3, thereby preparing a liquid resin composition. The obtained resin composition was spin-coated (rotation speed: 500 rpm, 30 seconds) on a glass substrate to form a coating film, and the obtained coating film was dried at 110° C. for 2 minutes to produce a coated and dried product (resin film) having a film thickness of 10 μm.

### <Test Example 5> Evaluation of Light Resistance of Coated and Dried Product

With regard to the coated and dried product produced above, a retention rate of absorbance at the maximal absorption wavelength (λmax) was obtained under the following (Condition 1), and the light resistance was evaluated. Specifically, after measuring the absorbance of the coated and dried product at the maximal absorption wavelength (λmax), the coated and dried product was subjected to a light resistance test after being irradiated for 50 hours under the following (Condition 2), and the absorbance at the maximal absorption wavelength (λmax) of the coated and dried product after the light resistance test was measured. The rate of change in absorbance at the maximal absorption wavelength (λmax) was calculated from the following expression. The results are shown in Table 3.

Rate of change in absorbance (%)=[(absorbance at λmax after 50 hour irradiation)/(absorbance at λmax before 50 hour irradiation)]×100

(Condition 1)

Using a spectrophotometer UV1900 (Shimadzu Corporation), the absorbance was measured at wavelength intervals of 1 nm in a wavelength range of 300 to 1000 nm for the glass substrate on which the coating film was formed.

(Condition 2)

Equipment: Xenon weather meter (Suga Test Instruments Co., Ltd.: XL75)

Illuminance: 100,000 lx (40 w/m²)

Test period: 50 hours

Environment: 23° C., relative humidity: 50%

### <Test Example 6> Evaluation of Surface Condition of Coated and Dried Product

The surface condition of the coated and dried product was evaluated in the same manner as in <Test Example 4> described above. The results are shown in Table 3.

### Examples 101 to 106 and 108

Coated and dried products of Examples 101 to 106 and 108 were produced in the same manner as in Example 107, except that the squarylium compound used in Example 107 and the content (parts by mass) thereof were changed to the contents shown in Table 3. The thickness of each coated and dried product was also set to be the same as the thickness of the coated and dried product of Example 107.

With regard to each of the produced coated and dried products, the light resistance and the surface condition were evaluated in the same manner as in Test Example 5 and Test Example 6 described above, and the results thereof are shown in Table 3.

TABLE 3

| | | | | Light | |
|---|---|---|---|---|---|
| | | Squarylium compound | | resistance | Surface |
| | Resin | Type | Content | (%) | condition |
| Example 101 | Resin 3 | A-4 | 0.08 | 85 | A |
| Example 102 | Resin 3 | A-12 | 0.05 | 88 | A |
| Example 103 | Resin 3 | A-19 | 0.15 | 89 | A |
| Example 104 | Resin 3 | A-28 | 0.14 | 89 | A |
| Example 105 | Resin 3 | B-2 | 0.12 | 85 | A |
| Example 106 | Resin 3 | B-11 | 0.05 | 87 | A |
| Example 107 | Resin 3 | B-12 | 0.07 | 88 | A |
| Example 108 | Resin 3 | B-20 | 0.19 | 86 | A |

### [Example D] Preparation of Resin Composition an Production an Evaluation of Optical Filter

The resin composition (molten mixture) according to the embodiment of the present invention was prepared using the squarylium compound synthesized in Example A and a polycarbonate resin as a resin 4 to produce an optical filter, and it was evaluated whether a precipitate of the squarylium compound was present in the obtained optical filter.

### Examples 201 to 208

1 kg of a polycarbonate resin (SD POLYCA 301-30 (trade name), glass transition point: 145° C. to 150° C., manufactured by Sumika Polycarbonate Limited) and 0.4 g of the squarylium compound shown in Table 4 were stirred in a stainless steel tumbler for 1 hour to obtain a mixture. Using a twin-screw kneading extruder (KZW15TW-45/60MG-NH (trade name), manufactured by TECHNOVEL CORPORATION), the obtained mixture was melt-kneaded at 280° C. to 320° C. for 1 minute to obtain a pellet-like melt-kneaded product. The obtained pellet-like melt-kneaded product was dried at 80° C. for 3 hours, and then molded with a press machine to produce a molded plate having a thickness of 0.15 mm.

### Test Example 7

The presence or absence of precipitate of the squarylium compound was visually observed in each of the produced molded plates (polycarbonate films).

—Evaluation Standard—

A: no precipitate

B: with precipitate

TABLE 4

| | | Presence or |
|---|---|---|
| | | absence of |
| | Squarylium compound | precipitate |
| Example 201 | A-4 | A |
| Example 202 | A-12 | A |
| Example 203 | A-19 | A |
| Example 204 | A-28 | A |
| Example 205 | B-2 | A |
| Example 206 | B-11 | A |
| Example 207 | B-12 | A |
| Example 208 | B-20 | A |

### [Example E] Preparation of Resin Composition and Production and Evaluation of Optical Filter

The resin composition (molten mixture) according to the embodiment of the present invention was prepared using the squarylium compound synthesized in Example A and a polyethylene terephthalate resin as a resin 5 to produce an optical filter, and it was evaluated whether a precipitate of the squarylium compound was present in the obtained optical filter.

### Examples 301 to 308

500 g of polyethylene terephthalate (TRN-8550F (trade name), melting point: 252° C., manufactured by TEIJIN LIMITED.) and 0.4 g of the squarylium compound shown in Table 5 were stirred in a stainless steel tumbler for 1 hour to obtain a mixture. The obtained mixture was melt-kneaded at 270° C. to obtain a pellet-like melt-kneaded product. The obtained pellet-like melt-kneaded product was dried at 80° C. for 3 hours, and then molded with a press machine to produce a molded plate having a thickness of 0.15 mm.

### Test Example 8

The presence or absence of precipitate of the squarylium compound was visually observed in each of the produced molded plates (PET films).

—Evaluation Standard—

A: no precipitate

B: with precipitate

TABLE 5

| | | Presence or |
|---|---|---|
| | | absence of |
| | Squarylium compound | precipitate |
| Example 301 | A-4 | A |
| Example 302 | A-12 | A |
| Example 303 | A-19 | A |
| Example 304 | A-28 | A |
| Example 305 | B-2 | A |
| Example 306 | B-11 | A |
| Example 307 | B-12 | A |
| Example 308 | B-20 | A |

From the results shown in Tables 1 to 5, the following was found.

Since the comparative compounds C-1 to C-3, C-5, and C-6 did not exhibit solubility in an organic solvent, and even in the case of the comparative compound C-4 exhibiting solubility in an organic solvent, it was found that the optical filters containing these comparative compounds could not achieve both light resistance and surface condition. It is considered that this is because the comparative compounds C-1 and C-4 satisfy the groups which can be represented as R¹ to R⁴ in Formula (1), but do not have any branched alkyl group having 4 or more carbon atoms. In addition, it is considered that this is because the comparative compound C-2 has all of phenyl groups as R¹ to R⁴ in Formula (1), and does not have any branched alkyl group having 4 or more carbon atoms. It is considered that this is because the comparative compound C-3 satisfies the groups which can be represented as R¹ to R⁴ in Formula (1), but does not have any branched alkyl group having 4 or more carbon atoms and has hydroxyl groups as R⁵ and R⁶. It is considered that this is because Comparative compound C-5 has a metallocene structural part in the molecule and has all of methyl groups as R¹ to R⁴ in Formula (4), but does not have the branched alkyl group having 4 or more carbon atoms, so that the solubility is deteriorated. It is considered that this is because, in the comparative compound C-6, all of R² and R⁴ in Formula (1) and Formula (2) are alkyl having 4 carbon atoms, but they are no branched but linear. In particular, since the comparative compounds C-1 to C-3 and C-5 have low solubility and easily form an associate, the optical filters thereof are significantly deteriorated in terms of surface condition.

On the other hand, all of the squarylium compounds according to the present invention, represented by Formula (1) or Formula (3) described above, have a maximal absorption wavelength in a wavelength range of 670 to 740 nm, and exhibit sufficient solubility in an organic solvent. In addition, the optical filters according to the embodiment of the present invention, containing these squarylium compounds, exhibit excellent surface condition (with little variation during film formation) regardless of the production method thereof, and provide a uniform film-like filter. Therefore, the optical filters containing these squarylium compounds can allow incidence light to enter the filter without reflection, can specifically absorb and block light in a specific wavelength range as unnecessary wavelength light, and exhibit a higher rate of change in absorbance (light resistance) than the optical filters of Comparative Examples. Moreover, even in a case where the squarylium compound according to the present invention is contained at a high concentration, it was found that it is possible to realize a resin composition free from deposits, precipitates, and the like due to aggregation (association) of the squarylium compounds, and realize an optical filter which can specifically absorb and block light in a specific wavelength range.

Therefore, an image display device equipped with the optical filter according to the embodiment of the present invention exhibits excellent light resistance, a wide color reproduction range, and spectral characteristics close to a spectral luminosity curve especially on a long wavelength side, and a solid-state imaging element including the optical filter according to the embodiment of the present invention is expected to exhibit excellent light resistance and excellent color reproducibility. In addition, the optical filter according to the embodiment of the present invention has excellent transmittance in a range of 400 to 600 nm, and is excellent in oblique incidence characteristics because it does not depend on the incidence angle, so that the optical filter according to the embodiment of the present invention can also be suitably used as a near-infrared cut filter with high light resistance.

The present invention has been described with the embodiments thereof, any details of the description of the present invention are not limited unless described otherwise, and it is obvious that the present invention is widely construed without departing from the gist and scope of the present invention described in the accompanying claims.

### EXPLANATION OF REFERENCES

1: upper polarizing plate
2: direction of absorption axis of upper polarizing plate
3: liquid crystal cell upper electrode substrate
4: controlled alignment direction of liquid crystal cell upper electrode substrate
5: liquid crystal layer
6: liquid crystal cell lower electrode substrate
7: controlled alignment direction of liquid crystal cell lower electrode substrate
8: lower polarizing plate
9: direction of absorption axis of lower polarizing plate
B: backlight unit
10: liquid crystal display device

What is claimed is:

1. A resin composition comprising:
a squarylium compound; and
a resin,
wherein the squarylium compound includes at least one selected from a squarylium compound represented by Formula (1) or a squarylium compound represented by Formula (3), 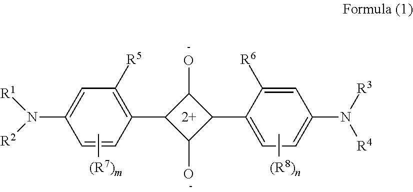
in Formula (1), R¹ to R⁴ represent an alkyl group or an aryl group, which may have a substituent, where at least one of R¹, . . . , or R⁴ is an aryl group and at least one of R¹, . . . , or R⁴ is an alkyl group, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3,
here, the squarylium compound represented by Formula (1) has at least one branched alkyl group having 4 or more carbon atoms,
Dye-(Q¹)ₙ₁ Formula (3)
in Formula (3), Dye represents a structural part obtained by removing n1 hydrogen atoms from a squarylium compound represented by Formula (4), Q¹ represents a group represented by Formula (4M), and n1 represents an integer of 1 to 6, 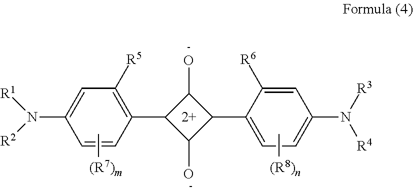
in Formula (4), R¹ to R⁴ represent an alkyl group or an aryl group, which may have a substituent, where at least one of R¹, . . . , or R⁴ is an aryl group and at least one of R¹, . . . , or R⁴ is an alkyl group, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3, 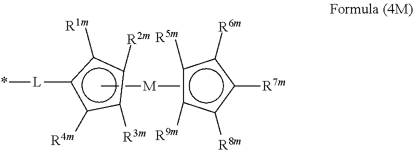
in Formula (4M), L represents a single bond or a divalent linking group which is not conjugated with Dye, R^{1m} to R^{9m} represent a hydrogen atom or a substituent, M represents Fe, Co, Ni, Ti, Cu, Zn, Zr, Cr, Mo, Os, Mn, Ru, Sn, Pd, Rh, V, or Pt, and * represents a bonding part with Dye.

2. The resin composition according to claim 1,
wherein the squarylium compound represented by Formula (1) is represented by Formula (2), 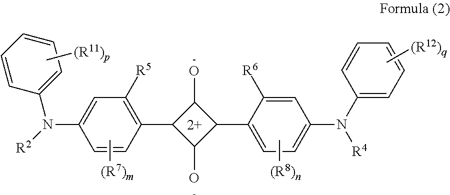
in Formula (2), R² and R⁴ represent an alkyl group, R¹¹ and R¹² represent a substituent, p and q represent an integer of 0 to 5, and R⁵ to R⁸, m, and n have the same meaning as R⁵ to R⁸, m, and n in Formula (1),
here, the squarylium compound represented by Formula (2) has at least one branched alkyl group having 4 or more carbon atoms.

3. The resin composition according to claim 1,
wherein at least one of R², R⁴, R⁹, or R¹⁰ contains a branched alkyl group having 4 or more carbon atoms.

4. The resin composition according to claim 1,
wherein the squarylium compound represented by Formula (4) is represented by Formula (5), 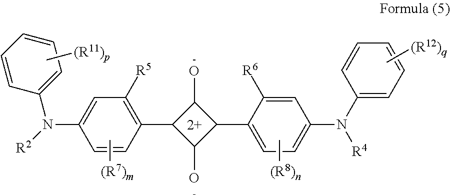
in Formula (5), R² and R⁴ represent an alkyl group, R¹¹ and R¹² represent a substituent, p and q represent an integer of 0 to 5, and R⁵ to R⁸, m, and n have the same meaning as R⁵ to R⁸, m, and n in Formula (4).

5. The resin composition according to claim 1,
wherein the squarylium compound represented by Formula (4) has at least one branched alkyl group having 4 or more carbon atoms.

6. The resin composition according to claim 1,
wherein M in Formula (4M) is Fe.

7. The resin composition according to claim 1,
wherein a glass transition temperature of the resin is −80° C. to 200° C.

8. The resin composition according to claim 1,
wherein the resin is at least one selected from a polystyrene resin, a cellulose acylate resin, a poly(meth)acrylic resin, a polyester resin, a cycloolefin resin, or a polycarbonate resin.

9. The resin composition according to claim 1, further comprising:
a solvent having a boiling point of 200° C. or lower,
wherein the resin and the squarylium compound are dissolved in the solvent.

10. An optical filter comprising the resin composition according to claim 1.

11. The optical filter according to claim 10, wherein the optical filter has a film form.

12. An image display device comprising the optical filter according to claim 10.

13. A solid-state imaging element comprising the optical filter according to claim 10.

14. A coated and dried product obtained by applying and drying the resin composition according to claim 9 on a substrate.

15. An optical filter comprising the coated and dried product according to claim 14.

16. The optical filter according to claim 15, wherein the optical filter has a film form.

17. An image display device comprising the optical filter according to claim 15.

18. A solid-state imaging element comprising the optical filter according to claim 15.

19. A melt-kneaded product of the resin composition according to claim 1.

20. An optical filter comprising the melt-kneaded product according to claim 19.

21. The optical filter according to claim 20, wherein the optical filter has a film form.

22. An image display device comprising the optical filter according to claim 20.

23. A solid-state imaging element comprising the optical filter according to claim 20.

24. A squarylium compound represented by Formula (1) or Formula (3), 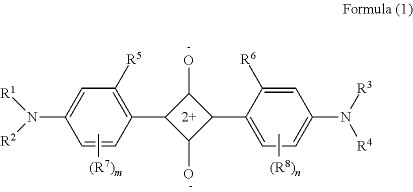
in Formula (1), R¹ to R⁴ represent an alkyl group or an aryl group, which may have a substituent, where at least one of R¹, . . . , or R⁴ is an aryl group and at least one of R¹, . . . , or R⁴ is an alkyl group, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3,
here, the squarylium compound represented by Formula (1) has at least one branched alkyl group having 4 or more carbon atoms,
Dye-(Q¹)ₙ₁ Formula (3)
in Formula (3), Dye represents a structural part obtained by removing n1 hydrogen atoms from a squarylium compound represented by Formula (4), Q¹ represents a group represented by Formula (4M), and n1 represents an integer of 1 to 6, 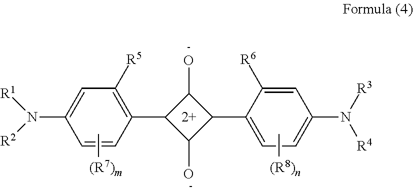
in Formula (4), R¹ to R⁴ represent an alkyl group or an aryl group, which may have a substituent, where at least one of R¹, . . . , or R⁴ is an aryl group and at least one of R¹, . . . , or R⁴ is an alkyl group, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3, 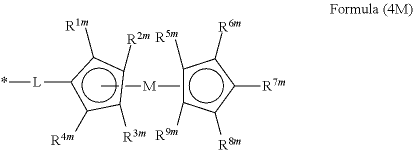
in Formula (4M), L represents a single bond or a divalent linking group which is not conjugated with Dye, R^{1m} to R^{9m} represent a hydrogen atom or a substituent, M represents Fe, Co, Ni, Ti, Cu, Zn, Zr, Cr, Mo, Os, Mn, Ru, Sn, Pd, Rh, V, or Pt, and * represents a bonding part with Dye.

25. The squarylium compound according to claim 24,
wherein the squarylium compound represented by Formula (1) is represented by Formula (2), 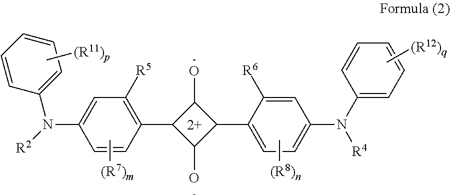
in Formula (2), R² and R⁴ represent an alkyl group, R¹¹ and R¹² represent a substituent, p and q represent an integer of 0 to 5, and R⁵ to R⁸, m, and n have the same meaning as R⁵ to R⁸, m, and n in Formula (1),
here, the squarylium compound represented by Formula (2) has at least one branched alkyl group having 4 or more carbon atoms.

26. The squarylium compound according to claim 24,
wherein the squarylium compound represented by Formula (4) is represented by Formula (5), 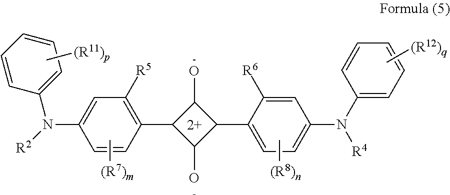
in Formula (5), R² and R⁴ represent an alkyl group, R¹¹ and R¹² represent a substituent, p and q represent an integer of 0 to 5, and R⁵ to R⁸, m, and n have the same meaning as R⁵ to R⁸, m, and n in Formula (4).

27. A method for producing a squarylium compound, comprising:
reacting a compound represented by Formula (A) with squaric acid or a compound represented by Formula (B) to produce a squarylium compound represented by Formula (1), 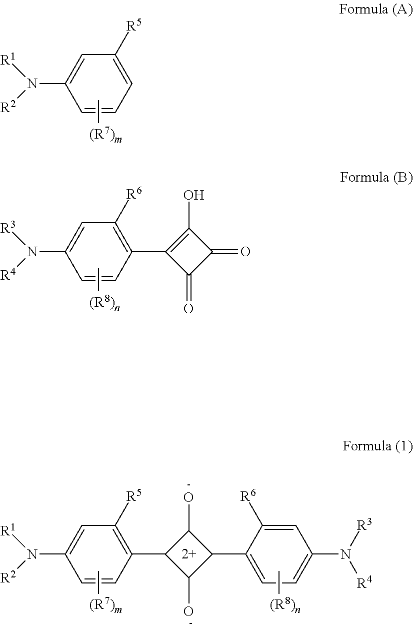
in Formula (A), Formula (B), and Formula (1), R¹ to R⁴ represent an alkyl group or an aryl group, which may have a substituent, R⁵ and R⁶ represent —NR⁹R¹⁰, where R⁹ and R¹⁰ represent a hydrogen atom, —COR^{N}, —COOR^{N}, —CON(R^{N})₂, or —SO₂R^{N}, and R^{N} represents a hydrogen atom or an alkyl group or an aryl group, which may have a substituent, R⁷ and R⁸ represent a substituent, and m and n represent an integer of 0 to 3,
here, in the compound represented by Formula (A) to be reacted with the squaric acid, at least one of R¹ or R² is an aryl group, at least one of R¹ or R² is an alkyl group, and the compound represented by Formula (A) has at least one branched alkyl group having 4 or more carbon atoms,
in the compounds represented by Formula (A) or Formula (B) to be reacted with each other, at least one of R¹, . . . , or R⁴ is an aryl group, at least one of R¹, . . . , or R⁴ is an alkyl group, and the compound represented by Formula (A) or Formula (B) has at least one branched alkyl group having 4 or more carbon atoms, and
the squarylium compound represented by Formula (1) has at least one branched alkyl group having 4 or more carbon atoms.
